SEQUENCE LISTING (102 sequences)

SEQ ID NO: 1 - protein, 10 aa, Homo sapiens
ALWGPDPAAA

SEQ ID NO: 2 - protein, 195 aa, Synthetic construct
QKEVEQNSGPLSVPEGAIASLNCTYSDRGSQSFFWYRQYSGKSPELIMSIYSNGDKEDGRFTAQLNKASQYVSLLIRDSQPSDSATYLCAVRGNEKLTFGTGTRLTIIPNIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 3 - protein, 109 aa, Synthetic construct
QKEVEQNSGPLSVPEGAIASLNCTYSDRGSQSFFWYRQYSGKSPELIMSIYSNGDKEDGRFTAQLNKASQYVSLLIRDSQPSDSATYLCAVRGNEKLTFGTGTRLTIIP

SEQ ID NO: 4 - protein, 86 aa, Synthetic construct
NIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 5 - protein, 6 aa, Synthetic construct
DRGSQS

SEQ ID NO: 6 - protein, 6 aa, Synthetic construct
IYSNGD

SEQ ID NO: 7 - protein, 9 aa, Synthetic construct
AVRGNEKLT

SEQ ID NO: 8 - protein, 26 aa, Synthetic construct
QKEVEQNSGPLSVPEGAIASLNCTYS

SEQ ID NO: 9 - protein, 17 aa, Synthetic construct
FFWYRQYSGKSPELIMS

SEQ ID NO: 10 - protein, 34 aa, Synthetic construct
KEDGRFTAQLNKASQYVSLLIRDSQPSDSATYLC

SEQ ID NO: 11 - protein, 11 aa, Synthetic construct
FGTGTRLTIIP

SEQ ID NO: 12 - protein, 231 aa, Synthetic construct
NAGVTQTPKFRILKIGQSMTLQCAQDMNHNYMYWYRQDPGMGLKPIYYSVGAGITDKGEVPNGYNVSRSTTEDFPLRLESAAPSQTSVYFCASSYMTGELFFGEGSRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFQDRAKPVTQIVSAEAWGRAD

SEQ ID NO: 13 - protein, 111 aa, Synthetic construct
NAGVTQTPKFRILKIGQSMTLQCAQDMNHNYMYWYRQDPGMGLKPIYYSVGAGITDKGEVPNGYNVSRSTTEDFPLRLESAAPSQTSVYFCASSYMTGELFFGEGSRLTVL

SEQ ID NO: 14 - protein, 120 aa, Synthetic construct
EDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFQDRAKPVTQIVSAEAWGRAD

SEQ ID NO: 15 - protein, 5 aa, Synthetic construct
MNHNY

SEQ ID NO: 16 - protein, 6 aa, Synthetic construct
SVGAGI

SEQ ID NO: 17 - protein, 10 aa, Synthetic construct
ASSYMTGELF

SEQ ID NO: 18 - protein, 26 aa, Synthetic construct
NAGVTQTPKFRILKIGQSMTLQCAQD

SEQ ID NO: 19 - protein, 17 aa, Synthetic construct
MYWYRQDPGMGLKPIYY

SEQ ID NO: 20 - protein, 37 aa, Synthetic construct
TDKGEVPNGYNVSRSTTEDFPLRLESAAPSQTSVYFC

SEQ ID NO: 21 - protein, 10 aa, Synthetic construct
FGEGSRLTVL

SEQ ID NO: 22 - protein, 109 aa, Synthetic construct
QKEVEQNSGPLSVPEGAIASLNCTYSDKHSQGFFWYRQYSGKSPELIMSIYSNGDKEDGRFTAQLNKASQYVSLLIRDSQPSDSATYLCAVRGNEKLTFGTGTRLTIIP

SEQ ID NO: 23 - protein, 6 aa, Synthetic construct
DKHSQG

SEQ ID NO: 24 - protein, 109 aa, Synthetic construct
AKEVEQNSGPLSVPEGAIASLQCTYSDKHSQGFFWYRQYSGKSPELIMSIYSNGDKEDGRFTAQLNKASQYVSLLIRDSQPSDSATYLCAVRGNEKLTFGTGTRLTIIP

SEQ ID NO: 25 - protein, 26 aa, Synthetic construct
AKEVEQNSGPLSVPEGAIASLQCTYS

SEQ ID NO: 26 - protein, 109 aa, Synthetic construct
AKEVEQNSGPLSVPEGAIASLQCTYSDKHSQGFFWYRQYSGKSPELIMSIYSQGDKEDGRFTAQLNKASQYVSLLIRDSQPSDSATYLCAVRGNEKLTFGTGTRLTIIP

SEQ ID NO: 27 - protein, 6 aa, Synthetic construct
IYSQGD

SEQ ID NO: 28 - protein, 5 aa, Synthetic construct
MNHSY

SEQ ID NO: 29 - protein, 6 aa, Synthetic construct
SVGVGF

SEQ ID NO: 30 - protein, 10 aa, Synthetic construct
ASAYMTGELF

SEQ ID NO: 31 - protein, 111 aa, Synthetic construct
NAGVTQTPKFRILKIGQSMTLQCAQDMQHSYMYWYRQDPGMGLKPIYYSVGVGFTDKGEVPNGYQVSRSTTEDFPLRLESAAPSQTSVYFCASAYMTGELFFGEGSRLTVL

SEQ ID NO: 32 - protein, 5 aa, Synthetic construct
MQHSY

SEQ ID NO: 33 - protein, 37 aa, Synthetic construct
TDKGEVPNGYQVSRSTTEDFPLRLESAAPSQTSVYFC

SEQ ID NO: 34 - protein, 111 aa, Synthetic construct
NAGVTQTPKFRILKIGQSMTLQCAQDLQHSYMYWYRQDPGMGLKPIYYSVGVGFTDKGEVPNGYQVSRSTTEDFPLRLESAAPSQTSVYFCASAYMTGELFFGEGSRLTVL

SEQ ID NO: 35 - protein, 5 aa, Synthetic construct
LQHSY

SEQ ID NO: 36 - protein, 195 aa, Synthetic construct
AKEVEQNSGPLSVPEGAIASLQCTYSDKHSQGFFWYRQYSGKSPELIMSIYSNGDKEDGRFTAQLNKASQYVSLLIRDSQPSDSATYLCAVRGNEKLTFGTGTRLTIIPNIQNPDPAVYQLRDSKSSDKSVCLFTDFDSQTQVSQSKDSDVYITDKCVLDMRSMDFKSNSAVAWSQKSDFACANAFQNSIIPEDT

SEQ ID NO: 37 - protein, 86 aa, Synthetic construct
NIQNPDPAVYQLRDSKSSDKSVCLFTDFDSQTQVSQSKDSDVYITDKCVLDMRSMDFKSNSAVAWSQKSDFACANAFQNSIIPEDT

SEQ ID NO: 38 - protein, 241 aa, Synthetic construct
NAGVTQTPKFRILKIGQSMTLQCAQDMQHSYMYWYRQDPGMGLKPIYYSVGVGFTDKGEVPNGYQVSRSTTEDFPLRLESAAPSQTSVYFCASAYMTGELFFGEGSRLTVLEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 39 - protein, 130 aa, Synthetic construct
EDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 40 - protein, 195 aa, Synthetic construct
AKEVEQNSGPLSVPEGAIASLQCTYSDKHSQGFFWYRQYSGKSPELIMSIYSQGDKEDGRFTAQLNKASQYVSLLIRDSQPSDSATYLCAVRGNEKLTFGTGTRLTIIPNIQNPDPAVYQLRDSKSSDKSVCLFTDFDSQTQVSQSKDSDVYITDKCVLDMRSMDFKSNSAVAWSQKSDFACANAFQNSIIPEDT

SEQ ID NO: 41 - protein, 241 aa, Synthetic construct
NAGVTQTPKFRILKIGQSMTLQCAQDLQHSYMYWYRQDPGMGLKPIYYSVGVGFTDKGEVPNGYQVSRSTTEDFPLRLESAAPSQTSVYFCASAYMTGELFFGEGSRLTVLEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 42 - protein, 117 aa, Synthetic construct
EVQLVESGGALVQPGGSLRLSCAASGFTFSSYAMTWVRQAPGKGPEWVSAIASDGASTSYLDSVKGRFTVSRDNAKNTLYLQMNSLKPEDTAVYYCARGGYLTYDRYGQGTQVTVSS

SEQ ID NO: 43 - protein, 8 aa, Synthetic construct
GFTFSSYA

SEQ ID NO: 44 - protein, 8 aa, Synthetic construct
IASDGAST

SEQ ID NO: 45 - protein, 12 aa, Synthetic construct
CARGGYLTYDRY

SEQ ID NO: 46 - protein, 363 aa, Synthetic construct
AVQLVESGGGLVQPGGSLRLSCAASGFTFSSYAMTWVRQAPGKGPEWVSAIASDGASTSYADSVKGRFTISRDNSKNTLYLQMNSLRPEDTAVYYCARGGYLTYDRYGQGTLVTVSSGGGGSNAGVTQTPKFRILKIGQSMTLQCAQDMQHSYMYWYRQDPGMGLKPIYYSVGVGFTDKGEVPNGYQVSRSTTEDFPLRLESAAPSQTSVYFCASAYMTGELFFGEGSRLTVLEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 47 - protein, 363 aa, Synthetic construct
AVQLVESGGGLVQPGGSLRLSCAASGFTFSSYAMTWVRQAPGKGPEWVSAIASDGASTSYADSVKGRFTISRDNSKNTLYLQMNSLRPEDTAVYYCARGGYLTYDRYGQGTLVTVSSGGGGSNAGVTQTPKFRILKIGQSMTLQCAQDLQHSYMYWYRQDPGMGLKPIYYSVGVGFTDKGEVPNGYQVSRSTTEDFPLRLESAAPSQTSVYFCASAYMTGELFFGEGSRLTVLEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 48 - protein, 422 aa, Synthetic construct
AKEVEQNSGPLSVPEGAIASLQCTYSDKHSQGFFWYRQYSGKSPELIMSIYSNGDKEDGRFTAQLNKASQYVSLLIRDSQPSDSATYLCAVRGNEKLTFGTGTRLTIIPNIQNPDPAVYQLRDSKSSDKSVCLFTDFDSQTQVSQSKDSDVYITDKCVLDMRSMDFKSNSAVAWSQKSDFACANAFQNSIIPEDTDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 49 - protein, 217 aa, Synthetic construct
APELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 50 - protein, 10 aa, Synthetic construct
DKTHTCPPCP

SEQ ID NO: 51 - protein, 227 aa, Synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 217 aa, Synthetic construct
APELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 422 aa, Synthetic construct; identical to SEQ ID NO: 48

SEQ ID NO: 54 - protein, 422 aa, Synthetic construct
AKEVEQNSGPLSVPEGAIASLQCTYSDKHSQGFFWYRQYSGKSPELIMSIYSQGDKEDGRFTAQLNKASQYVSLLIRDSQPSDSATYLCAVRGNEKLTFGTGTRLTIIPNIQNPDPAVYQLRDSKSSDKSVCLFTDFDSQTQVSQSKDSDVYITDKCVLDMRSMDFKSNSAVAWSQKSDFACANAFQNSIIPEDTDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 55 - protein, 5 aa, Synthetic construct
GGGSG

SEQ ID NO: 56 - protein, 5 aa, Synthetic construct
GGSGG

SEQ ID NO: 57 - protein, 5 aa, Synthetic construct
GSGGG

SEQ ID NO: 58 - protein, 6 aa, Synthetic construct
GSGGGP

SEQ ID NO: 59 - protein, 5 aa, Synthetic construct
GGEPS

SEQ ID NO: 60 - protein, 7 aa, Synthetic construct
GGEGGGP

SEQ ID NO: 61 - protein, 12 aa, Synthetic construct
GGEGGGSEGGGS

SEQ ID NO: 62 - protein, 8 aa, Synthetic construct
GGGSGGGG

SEQ ID NO: 63 - protein, 24 aa, Synthetic construct
GGGGSGGGGSGGGGSGGGGSGGGS

SEQ ID NO: 64 - protein, 20 aa, Synthetic construct
GGGGSGGGGSGGGGSGGGGS

SEQ ID NO: 65 - protein, 5 aa, Synthetic construct
EAAAK

SEQ ID NO: 66 - protein, 15 aa, Synthetic construct
EAAAKEAAAKEAAAK

SEQ ID NO: 67 - protein, 10 aa, Homo sapiens
ALLGPDPAAA

SEQ ID NO: 68 - protein, 111 aa, Synthetic construct
NAGVTQTPKFRILKIGQSMTLQCAQDMNHSYMYWYRQDPGMGLKPIYYSVGVGFTDKGEVPNGYNVSRSTTEDFPLRLESAAPSQTSVYFCASAYMTGELFFGEGSRLTVL

SEQ ID NO: 69 - protein, 241 aa, Synthetic construct
NAGVTQTPKFRILKIGQSMTLQCAQDMNHSYMYWYRQDPGMGLKPIYYSVGVGFTDKGEVPNGYNVSRSTTEDFPLRLESAAPSQTSVYFCASAYMTGELFFGEGSRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 70 - protein, 195 aa, Synthetic construct
QKEVEQNSGPLSVPEGAIASLNCTYSDKHSQGFFWYRQYSGKSPELIMSIYSNGDKEDGRFTAQLNKASQYVSLLIRDSQPSDSATYLCAVRGNEKLTFGTGTRLTIIPNIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 71 - protein, 117 aa, Synthetic construct
AVQLVESGGGLVQPGGSLRLSCAASGFTFSSYAMTWVRQAPGKGPEWVSAIASDGASTSYADSVKGRFTISRDNSKNTLYLQMNSLRPEDTAVYYCARGGYLTYDRYGQGTLVTVSS

SEQ ID NO: 72 - protein, 363 aa, Synthetic construct; identical to SEQ ID NO: 46

SEQ ID NO: 73 - protein, 5 aa, Synthetic construct
GGGGS

SEQ ID NO: 74 - protein, 111 aa, Synthetic construct
NAGVTQTPKFRILKIGQSMTLQCAQDLQHSYMYWYRQDPGMGLKPIYYSVGVGFTDKGEVPQGYQVSRSTTEDFPLRLESAAPSQTSVYFCASAYMTGELFFGEGSRLTVL

SEQ ID NO: 75 - protein, 37 aa, Synthetic construct
TDKGEVPQGYQVSRSTTEDFPLRLESAAPSQTSVYFC

SEQ ID NO: 76 - protein, 111 aa, Synthetic construct
NAGVTQTPKFRILKIGQSMTLQCAQDLQHSYMYWYRQDPGMGLKPIYYSVGVGFTDKGEVPEGYQVSRSTTEDFPLRLESAAPSQTSVYFCASAYMTGELFFGEGSRLTVL

SEQ ID NO: 77 - protein, 37 aa, Synthetic construct
TDKGEVPEGYQVSRSTTEDFPLRLESAAPSQTSVYFC

SEQ ID NO: 78 - protein, 111 aa, Synthetic construct
NAGVTQTPKFRILKIGQSMTLQCAQDLQHSYMYWYRQDPGMGLKPIYYSVGVGFTDKGEVPDGYQVSRSTTEDFPLRLESAAPSQTSVYFCASAYMTGELFFGEGSRLTVL

SEQ ID NO: 79 - protein, 37 aa, Synthetic construct
TDKGEVPDGYQVSRSTTEDFPLRLESAAPSQTSVYFC

SEQ ID NO: 80 - protein, 241 aa, Synthetic construct
NAGVTQTPKFRILKIGQSMTLQCAQDLQHSYMYWYRQDPGMGLKPIYYSVGVGFTDKGEVPQGYQVSRSTTEDFPLRLESAAPSQTSVYFCASAYMTGELFFGEGSRLTVLEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 81 - protein, 241 aa, Synthetic construct
NAGVTQTPKFRILKIGQSMTLQCAQDLQHSYMYWYRQDPGMGLKPIYYSVGVGFTDKGEVPEGYQVSRSTTEDFPLRLESAAPSQTSVYFCASAYMTGELFFGEGSRLTVLEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 82 - protein, 241 aa, Synthetic construct
NAGVTQTPKFRILKIGQSMTLQCAQDLQHSYMYWYRQDPGMGLKPIYYSVGVGFTDKGEVPDGYQVSRSTTEDFPLRLESAAPSQTSVYFCASAYMTGELFFGEGSRLTVLEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 83 - protein, 363 aa, Synthetic construct
AVQLVESGGGLVQPGGSLRLSCAASGFTFSSYAMTWVRQAPGKGPEWVSAIASDGASTSYADSVKGRFTISRDNSKNTLYLQMNSLRPEDTAVYYCARGGYLTYDRYGQGTLVTVSSGGGGSNAGVTQTPKFRILKIGQSMTLQCAQDLQHSYMYWYRQDPGMGLKPIYYSVGVGFTDKGEVPQGYQVSRSTTEDFPLRLESAAPSQTSVYFCASAYMTGELFFGEGSRLTVLEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 84 - protein, 363 aa, Synthetic construct
AVQLVESGGGLVQPGGSLRLSCAASGFTFSSYAMTWVRQAPGKGPEWVSAIASDGASTSYADSVKGRFTISRDNSKNTLYLQMNSLRPEDTAVYYCARGGYLTYDRYGQGTLVTVSSGGGGSNAGVTQTPKFRILKIGQSMTLQCAQDLQHSYMYWYRQDPGMGLKPIYYSVGVGFTDKGEVPEGYQVSRSTTEDFPLRLESAAPSQTSVYFCASAYMTGELFFGEGSRLTVLEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 85 - protein, 363 aa, Synthetic construct
AVQLVESGGGLVQPGGSLRLSCAASGFTFSSYAMTWVRQAPGKGPEWVSAIASDGASTSYADSVKGRFTISRDNSKNTLYLQMNSLRPEDTAVYYCARGGYLTYDRYGQGTLVTVSSGGGGSNAGVTQTPKFRILKIGQSMTLQCAQDLQHSYMYWYRQDPGMGLKPIYYSVGVGFTDKGEVPDGYQVSRSTTEDFPLRLESAAPSQTSVYFCASAYMTGELFFGEGSRLTVLEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 86 - protein, 13 aa, Synthetic construct
CAVGAGSARQLTF

SEQ ID NO: 87 - protein, 14 aa, Synthetic construct
CASSQRGTFGEQFF

SEQ ID NO: 88 - protein, 10 aa, Homo sapiens
ALPGPDEAAA

SEQ ID NO: 89 - protein, 10 aa, Homo sapiens
ALMSPPPAAA

SEQ ID NO: 90 - protein, 10 aa, Homo sapiens
ALWDPGPEAA

SEQ ID NO: 91 - protein, 10 aa, Homo sapiens
ALGSPPPAAA

SEQ ID NO: 92 - protein, 217 aa, Synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 217 aa, Synthetic construct
APELLGGPSVFLFPPKPKDTLYITREPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 94 - protein, 217 aa, Synthetic construct
APELLGGPSVFLFPPKPKDTLYITREPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 15 aa, Synthetic construct
EPKSSDKTHTCPPCP

SEQ ID NO: 96 - protein, 12 aa, Synthetic construct
ESKYGPPCPSCP

SEQ ID NO: 97 - protein, 422 aa, Synthetic construct
AKEVEQNSGPLSVPEGAIASLQCTYSDKHSQGFFWYRQYSGKSPELIMSIYSNGDKEDGRFTAQLNKASQYVSLLIRDSQPSDSATYLCAVRGNEKLTFGTGTRLTIIPNIQNPDPAVYQLRDSKSSDKSVCLFTDFDSQTQVSQSKDSDVYITDKCVLDMRSMDFKSNSAVAWSQKSDFACANAFQNSIIPEDTDKTHTCPPCPAPELLGGPSVFLFPPKPKDTLYITREPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 98 - protein, 227 aa, Synthetic construct
DKTHTCPPCPAPELLGGPSVFLFPPKPKDTLYITREPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 422 aa, Synthetic construct; identical to SEQ ID NO: 97

SEQ ID NO: 100 - protein, 422 aa, Synthetic construct
AKEVEQNSGPLSVPEGAIASLQCTYSDKHSQGFFWYRQYSGKSPELIMSIYSQGDKEDGRFTAQLNKASQYVSLLIRDSQPSDSATYLCAVRGNEKLTFGTGTRLTIIPNIQNPDPAVYQLRDSKSSDKSVCLFTDFDSQTQVSQSKDSDVYITDKCVLDMRSMDFKSNSAVAWSQKSDFACANAFQNSIIPEDTDKTHTCPPCPAPELLGGPSVFLFPPKPKDTLYITREPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 101 - protein, 147 aa, Synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 102 - protein, 115 aa, Synthetic construct
FTVTVPKDLYWEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPY

(19) United States
(12) Patent Application Publication
(10) Pub. No.: US 2026/0285984 A1
(43) Pub. Date: Sep. 24, 2026

(54) TCR WITH HIGH AFFINITY AND SPECIFICITY SPECIFIC FOR PREPROINSULIN PEPTIDE ALWGPDPAAA BOUND TO HLA-A2*02

(71) Applicant: IMMUNOCORE LIMITED, Abingdon, Oxfordshire (GB)

(72) Inventors: Tara MAHON, Abingdon, Oxfordshire (GB); David OVERTON, Abingdon, Oxfordshire (GB); Rita FIGUEIREDO, Abingdon, Oxfordshire (GB); Rajeevkumar TAWAR, Abingdon, Oxfordshire (GB); Katy WISEMAN, Abingdon, Oxfordshire (GB)

(21) Appl. No.: 19/478,708

(22) PCT Filed: Apr. 26, 2024
(86) PCT No.: PCT/EP2024/061568

§ 371 Date: Oct. 24, 2025

(30) Foreign Application Priority Data
Apr. 28, 2023 (GB) 2306345.6

(51) Int. Cl. C07K 16/28; A61K 38/00; A61K 39/00; A61K 40/11; A61K 40/32; A61K 40/41; C07K 14/705; C12N 5/0783

(52) U.S. Cl. CPC C07K 16/2833; A61K 40/11; A61K 40/32; A61K 40/41; C07K 14/7051; C07K 16/2818; C12N 5/0636; A61K 38/00; A61K 2039/505; C07K 2317/569; C07K 2317/92; C07K 2317/94; C07K 2319/00; C12N 2510/00

(57) ABSTRACT

The present invention relates to binding molecules comprising a peptide-major histocompatibility complex (pMHC)-binding domain which binds to a ALWGPDPAAA (SEQ ID NO: 1) peptide (derived from human pre-pro insulin (PPI) protein) presented as a peptide-HLA-A*02 complex. Said binding molecules may comprise CDR sequences embedded within a framework sequence. The CDRs and framework sequences may correspond to a T cell receptor (TCR) variable domain and may further comprise non-natural mutations relative to a native TCR variable domain. The binding molecules of the invention have high affinity and high specificity for the ALWGPDPAAA (SEQ ID NO: 1)-HLA-A*02 complex. The binding molecules may further comprise an immune suppressor and/or a half-life extending domain. Such binding molecules are particularly useful in the development of soluble immunotherapeutic reagents for the treatment of autoimmune diseases, such as diabetes.

### FIELD THE INVENTION

The present invention relates to binding molecules comprising a peptide-major histocompatibility complex (pMHC)-binding domain which comprises TCR variable domains. The binding molecules may further comprise an immune suppressor and/or a half-life extending domain. The invention also relates to the use of the binding molecules in the treatment or diagnosis of autoimmune diseases, such as diabetes.

### BACKGROUND TO THE INVENTION

Autoimmune diseases are often chronic and debilitating, and constitute an area of clinical need. Organ-specific immune suppression, rather than systemic immunosuppression, may be a beneficial route for treatment given the potential significant adverse events associated with systemic immunosuppression.

In autoimmunity, there is also mounting evidence that PD-1 pathway impairment plays an important role in disease pathogenesis. PD-1, PD-L1 and PD-L2 gene polymorphisms are associated with several autoimmune diseases. Abnormally low PD-L1 expression has been observed in samples from type 1 diabetes and Crohn's disease patients. Activating PD-1 on autoreactive lymphocytes thus may serve as a mechanism to treat autoimmune diseases. However, few PD-1 agonists have reached the clinic, and efficacy in patients is yet to be demonstrated.

Type 1 diabetes mellitus (T1DM) is an auto-immune disease characterised by metabolic dysfunction, most notably dysregulation of glucose metabolism, accompanied by characteristic long-term vascular and neurological complications. T1DM is characterised by absolute insulin deficiency, making patients dependent on exogenous insulin for survival. Prior to the acute clinical onset of T1DM with symptoms of hyperglycemia there is a long asymptomatic preclinical period, during which insulin-producing beta cells are progressively destroyed. The autoimmune destruction of beta cells (β cells) is associated with lymphocytic infiltration.

There is ample evidence that CD8⁺ T cells are involved in the disease process that leads to T1DM. Histological analysis of the islets in an affected individual shows infiltration by CD8⁺ T cells. In animal models of T1DM, the disease process may be transferred from a diseased animal to a healthy animal using CD8⁺ T cells.

A systemically acting immunotherapy, based on a blocking anti-CD3 antibody, recently received FDA approval for the treatment of T1DM stage 2 disease. However, the non-targeted nature of the drug leads to increased safety concerns, with a major side effect being lymphopenia in the early stages of treatment. An antigen-specific (or tissue-specific) immunotherapy of type 1 diabetes in the early, post-onset period has the potential to halt disease progression and preserve remaining islet cell function, while avoiding systemic immune inactivation. A safe immunotherapy could also be considered for the protection of islet allografts and for prophylaxis where strong genetic predisposition to type I diabetes is present. Islet beta cells are naturally protected from pathogenic T cells by Foxp3 expressing regulatory CD4⁺ T cells (Treg) and it is established that protection mediated by adoptively transferred T cells requires recognition of an islet cell antigen.

A number of diabetes-specific human auto-reactive CD8⁺ T cells have been isolated from diseased individuals (Skowera, et al. 2008 J Clin Invest. 118:3390-402 and Lieberman et al. Proc Natl Acad Sci U.S.A. 2003 Jul. 8; 100(14):8384-8). These T cells bear T cell receptors (TCRs) which primarily recognise peptide epitopes of β-cell antigens such as pre-pro-insulin (PPI). The ALWGPDPAAA₁₅₋₂₄ (SEQ ID No: 1) peptide is one such peptide derived from the signal sequence of human PPI (Skowera, et al. 2008 J Clin Invest. 118:3390-402 and WO2009004315). The peptide is loaded on to HLA-A*02 molecules and presented on the surface of insulin-producing B cells. Therefore, the ALWGPDPAAA-HLA-A*02 complex provides a human beta cell-specific marker that can be recognised by TCRs. High expression of this PPI peptide can be detected on the surface of beta cells, independent of disease stage, meaning a PPI targeted therapeutic could be efficacious at earlier disease stages compared to existing immunotherapies.

WO2015092362 discloses TCRs and fusion molecules that bind to the ALWGPDPAAA peptide-HLA-A*02 complex. WO2019219709 discloses a TCR that binds to the ALWGPDPAAA peptide-HLA-A*02 complex as well as such a TCR fused to PD1 agonists (either the natural ligand, PDL1, or an anti-PD1 scFv). Curnock et al, 2021, JCI Insight. 2021; 6(20):e15246 discloses bispecific molecules consisting of a soluble TCR that is specific for the ALWGPDPAAA peptide HLA-A*02 complex and an effector end comprising a PD1 agonist.

While TCRs isolated from human donors are generally deemed preferable from a safety point of view, for autoreactive TCRs, this may not be the case as TCRs that recognize autoantigens likely should have been deleted during thymic selection. In addition, autoreactive TCRs from patients generally have lower affinity than those specific for cancer or pathogen antigens (Dolton G. et al., Frontiers Imm. 2018), potentially due to their inability to dock correctly on HLA.

There remains a need to provide potent, specific, and effective tissue-targeted immunosuppressive compositions that are optimized for the diagnosis and treatment of autoimmune diseases, such as T1DM, where such immunosuppressive compositions avoid risks associated with systemic immunosuppression. Effective therapeutics for the treatment of autoimmune diseases include those having an advantageous risk profile (e.g., a high level of target and tissue specificity) and are capable of being administered with less frequency.

The production of a TCR engineered to have high affinity particularly when balanced with other desirable features is not straightforward, and typically has a high attrition rate. In the first instance, the skilled person needs to identify a suitable starting, or scaffold, sequence. Typically such sequences may be obtained from natural sources e.g. from antigen responding T cells extracted from donor blood, or from TCR libraries comprising alpha and beta chains obtained from a natural repertoire. Given the rarity of T cells in the natural repertoire that are specific for self-antigens, such as PPI, it is often necessary to screen many donors, for example 20 or more, before a responding T cell may be found. The screening process may take several weeks or months, and even where a responding T cell is found, it may be unsuitable for immunotherapeutic use. For example, the response may be too weak and/or may not be specific for the target antigen. Alternatively, it may not be possible to generate a clonal T cell population, nor expand or maintain a given T cell line to produce sufficient material to identify the correct TCR chain sequences. Likewise, it may not be possible to identify antigen specific TCRs from native libraries. TCR sequences that are suitable as starting, or scaffold, sequences should have one or more of the following properties: a good affinity for the target peptide-HLA complex, for example 200 μM or stronger; a high level of target specificity, e.g. relatively weak or no binding to alternative peptide-HLA complexes (which is especially important for the treatment of autoimmune conditions); be amenable to use in display libraries, such as phage display; be able to be refolded and/or purified at high yield from a relevant expression system; and maintain stability in purified form, including as a fusion protein. Given the degenerate nature of TCR recognition, it is exceptionally hard even for skilled practitioners to be able to determine whether a particular scaffold TCR sequence has a specificity profile that would make it eligible for engineering for therapeutic use (Wooldridge, et al., J Biol Chem. 2012 Jan. 6; 287(2):1168-77).

The next challenge is to engineer the TCR to have a higher affinity towards the target antigen whilst retaining desirable characteristics such as specificity and yield. TCRs, as they exist in nature, have weak affinity for target antigen (low micromolar range) compared with antibodies. This weak affinity means that therapeutic TCRs for immunotherapy typically require engineering to increase their affinity for target antigen and thus generate a more potent response. Such affinity increases are essential for soluble TCR-based reagents. In such cases, antigen-binding affinities in the nanomolar to picomolar range, with binding half-lives of several hours, are desirable. The improved potency generated by high affinity antigen recognition at low epitope numbers is exemplified in FIGS. 1e and 1f of Liddy et al. (Liddy, et al., Nat Med. 2012 June; 18(6):980-7). The affinity maturation process typically involves the skilled person having to engineer specific mutations, including but not limited to substitutions, insertions and/or deletions, on to the starting TCR sequence in order to increase the strength of antigen recognition. Affinity maturation techniques are known in the art, for example the use of display libraries (Li et al., Nat Biotechnol. 2005 March; 23(3):349-54; Holler et al., Proc Natl Acad Sci USA. 2000 May 9; 97(10):5387-92). However, to produce significant increases in the affinity of a given TCR against a given target, the skilled person may have to engineer mutations from a large pool of possible alternatives. The specific mutations that produce significant increases in affinity are not predictable and there is a high attrition rate. In many cases, it may not be possible to achieve significant increases in affinity with a given TCR starting sequence.

The affinity maturation process must also take account of the necessity of maintaining TCR antigen specificity. Increasing the affinity of a TCR for its target antigen brings a substantial risk of revealing cross reactivity with other unintended targets as a result of the inherent degeneracy of TCR antigen recognition (Wooldridge, et al., J Biol Chem. 2012 Jan. 6; 287(2):1168-77; Wilson, et al., Mol Immunol 2004, 40(14-15):1047-55; Zhao et al., J Immunol 2007, 179(9):5845-54). At a natural level of affinity the recognition of the cross reactive antigen may be too low to produce a response. If a cross reactive antigen is displayed on normal healthy cells, there is a strong possibility of off-target binding in vivo which may manifest in clinical toxicity. Thus, in addition to increasing antigen binding strength, the skilled person must also engineer mutations and/or combinations of mutations that allow the TCR to retain a high specificity for target antigen and demonstrate a good safety profile in preclinical testing. Again, suitable mutations and/or combinations of mutations are not predictable. The attrition rate at this stage is even higher and in many cases may not be achievable at all from a given TCR starting sequence.

### DESCRIPTION OF THE INVENTION

### Binding Molecules

In a first aspect, the present invention provides a binding molecule comprising a peptide-major histocompatibility complex (pMHC)-binding domain that has the property of binding to a ALWGPDAAA (SEQ ID NO: 1) HLA-A*02 complex, wherein the pMHC-binding domain comprises (i) an alpha chain, comprising at least a TCR alpha chain variable domain, and (ii) a beta chain, comprising at least a TCR beta chain variable domain, wherein
(a) the TCR alpha chain variable domain comprises a CDR1, a CDR2 and a CDR3 comprising the following sequences:
CDR1—DKHSQG (SEQ ID NO: 23), optionally with one, two or three mutations therein,
CDR2—IYSQGD (SEQ ID NO: 27), optionally with one, two or three mutations therein,
CDR3—AVRGNEKLT (SEQ ID NO: 7), optionally with one, two or three mutations therein, and/or
(b) the TCR beta chain variable domain comprises a CDR1, a CDR2 and a CDR3 comprising the following sequences:
CDR1—LQHSY (SEQ ID NO: 35), optionally with one, two or three mutations therein,
CDR2—SVGVGF (SEQ ID NO: 29), optionally with one, two or three mutations therein,
CDR3—ASAYMTGELF (SEQ ID NO: 30), optionally with one, two or three mutations therein.

The inventors have surprisingly identified binding molecules, comprising TCR variable domains, with a particularly high affinity (picomolar range), and a high degree of antigen specificity for the ALWGPDPAAA (SEQ ID NO: 1)-HLA-A*02 complex. Said molecules demonstrate potent protection of PPI positive cells when prepared as soluble reagents fused to an immune suppressor. The molecules of the invention thus have a particularly suitable profile for therapeutic use. Particular binding molecules of the invention were engineered from a suitable scaffold (i.e., “wildtype” or “native”) TCR sequence (comprising an alpha chain sequence of SEQ ID NO: 2 and a beta chain sequence of SEQ ID NO: 12) into which a number of mutations were introduced to enhance affinity, manufacturability and/or stability, while maintaining high specificity.

The binding molecules of the invention are distinct from earlier molecules shown to bind to the ALWGPDPAAA peptide-HLA-A*02 complex, such as those in WO2015092362, WO2019219709 and Curnock et al described above. For example, the binding molecules of the invention have increased specificity for the ALWGPDPAAA (SEQ ID NO: 1)-HLA-A*02 complex, and are produced with greater yield, relative to a TCR disclosed in WO2015092362 (see Example 3 herein). Also, certain binding molecules of the invention have been engineered to possess extended half-lives, to be suitable for treatment of autoimmune disease, whilst overcoming challenges to retain specificity and potency.

Therapeutic agents based on the binding molecules of the invention can be used for the purpose of delivering immunosuppressive agents to beta cells in order to prevent their destruction by CD8⁺ T cells. Such immunosuppressive agents include antibody fragments or cytokines.

Binding molecules of the invention can also be used in a treatment process known as adoptive therapy. For example, T regulatory cells (Tregs) transfected with MHC class I restricted TCRs, such as the binding molecules of the invention, may produce enhanced suppression of T effector cells compared with non-transfected Tregs (Plesa et al. 2012 Blood. 119(15):3420-3430) and such cells have significant potential in the treatment of autoimmune diseases (Wright et al. 2011 Expert Rev Clin Immunol. 7(2):213-25). Regulatory T cells (Treg) constitute a small proportion (5 to 10%) of the total population of CD4+ T lymphocytes (Powrie et al., (2003) Science 299 (5609): 1030-1) and are characterized by the constitutive expression of CD25 and the Foxp3 transcription factor.

Binding molecules of the invention may also be used as diagnostic reagents to detect cells presenting the ALWGPDPAAA (SEQ ID NO: 1)-HLA-A*02 complex. In this case the molecules may be fused to a detectable label.

To ensure effective targeting of ALWGPDPAAA (SEQ ID NO: 1)-HLA-A*02 presenting B cells, binding molecules of the present invention may have an improved binding affinity for, and/or binding half-life for, the peptide-HLA complex. It is desirable that certain binding molecules of the invention, such as those used to deliver therapeutic agents or in diagnosis, have a high affinity and/or a slow off-rate for the peptide-HLA complex. The inventors have also found that the binding molecules have a surprisingly high specificity for the ALWGPDPAAA (SEQ ID NO: 1)-HLA-A*02 complex.

The peptide ALWGPDPAAA (SEQ ID NO: 1) corresponds to amino acids 15-24 of human preproinsulin (Uniprot P01308).

As used herein, the term “binding molecule” generally refers to a molecule capable of binding to one or more target antigen(s). The binding molecule may comprise one or more polypeptide chain(s). As used herein, the term “polypeptide chain” refers to a polymer (i.e., a chain) of amino acids, typically twenty or more amino acids, linked by peptide bonds and having an N- and C-terminus. As is known in the art, a protein may comprise multiple polypeptide chains assembled together by non-covalent or covalent interactions.

The binding molecules of the invention comprise a “pMHC-binding domain”, which refers to a protein domain capable of binding to a peptide-MHC complex. The pMHC-binding domain comprises (a) an alpha chain comprising at least a TCR alpha chain variable domain and (b) a beta chain comprising at least a TCR beta chain variable domain. In this context the term “alpha chain” refers to the region of the binding molecule that comprises the TCR alpha chain variable domain and the term “beta chain” refers to the region of the binding molecule that comprises the TCR beta chain variable domain. The alpha chain and beta chain may be present in the same or different polypeptide chains within the binding molecule. The pMHC-binding domain may be, or comprise, a TCR, such as a soluble TCR.

Each of the TCR alpha chain variable domain and the TCR beta chain variable domain in the pMHC-binding domain comprises three CDRs and four framework regions arranged as FR1-CDR1-FR2-CDR2-FR3-CDR3-FR4, where FR is a framework region and CDR is a complementarity determining region.

The alpha chain and beta chain may or may not comprise TCR constant domains as described herein. The TCR variable domains associate together to form a TCR binding site which is capable of binding to the ALWGPDPAAA (SEQ ID NO: 1)-HLA-A*02 complex. Such molecules may adopt a number of different formats as discussed herein. Furthermore, fragments of the binding molecules of the invention are also envisioned. A fragment refers to a portion of the binding molecule that retains binding to the target antigen.

The binding molecules of the invention comprise TCR variable domains, which may correspond to those from a native TCR, or more preferably the TCR variable domains may be engineered (i.e., contain mutations relative to the native sequence). Native TCR variable domains may also be referred to as wild-type, natural, parental, unmutated or scaffold domains. The binding molecules of the invention may have ideal therapeutic properties such as supra-physiological affinity for target, long binding half-life, high specificity for target and good stability. The invention also includes multispecific (e.g., bispecific), or multifunctional (e.g., bifunctional), or fusion, molecules that incorporate TCR variable domains described herein and a therapeutic moiety such as, for example, an immunosuppressive agent. Such molecules can mediate a potent and specific protection of PPI positive cells by suppressing CD8⁺ T cells. Furthermore, the use of binding molecules with supra-physiological affinity facilitates recognition of such beta cells presenting low levels of the target peptide-HLA complex. Alternatively, the binding molecules may further comprise (e.g., by fusion) other therapeutic agents, and/or diagnostic agents.

The binding molecule of the invention may be in the form of a TCR, which comprises the TCR alpha chain variable domain and the TCR beta chain variable domain. The TCR may be a soluble TCR, i.e. a TCR that does not comprise a transmembrane domain and does not comprise an intracellular/cytoplasmic domain. The TCR domain sequences may be defined with reference to IMGT nomenclature which is widely known and accessible to those working in the TCR field. For example, see: LeFranc and LeFranc, (2001). “T cell Receptor Factsbook”, Academic Press; Lefranc, (2011), Cold Spring Harb Protoc 2011 (6): 595-603; Lefranc, (2001), Curr Protoc Immunol Appendix 1: Appendix 10; and Lefranc, (2003), Leukemia 17(1):260-266. Briefly, αβ TCRs consist of two disulphide linked chains. Each chain (alpha and beta) is generally regarded as having two domains, namely a variable and a constant domain. A short joining region connects the variable and constant domains and is typically considered part of the alpha variable region. Additionally, the beta chain usually contains a short diversity region next to the joining region, which is also typically considered part of the beta variable region. The variable domain of each chain is located N-terminally and comprises three Complementarity Determining Regions (CDRs) embedded in a framework sequence (FR). The CDRs comprise the recognition site for peptide-MHC binding. There are several genes coding for alpha chain variable (Va) regions and several genes coding for beta chain variable (VB) regions, which are distinguished by their framework, CDR1 and CDR2 sequences, and by a partly defined CDR3 sequence. The Vα and Vβ genes are referred to in IMGT nomenclature by the prefix TRAV and TRBV respectively (Folch and Lefranc, (2000), Exp Clin Immunogenet 17(1):42-54; Scaviner and Lefranc, (2000), Exp Clin Immunogenet 17(2):83-96; LeFranc and LeFranc, (2001), “T cell Receptor Factsbook”, Academic Press). Likewise there are several joining or J genes, termed TRAJ or TRBJ, for the alpha and beta chain respectively, and for the beta chain, a diversity or D gene termed TRBD (Folch and Lefranc, (2000), Exp Clin Immunogenet 17(2):107-114; Scaviner and Lefranc, (2000), Exp Clin Immunogenet 17(2):97-106; LeFranc and LeFranc, (2001), “T cell Receptor Factsbook”, Academic Press). The huge diversity of T cell receptor chains results from combinatorial rearrangements between the various V, J and D genes, which include allelic variants, and junctional diversity (Arstila, et al., (1999), Science 286(5441): 958-961; Robins et al., (2009), Blood 114(19): 4099-4107.) The constant, or C, regions of TCR alpha and beta chains are referred to as TRAC and TRBC respectively (Lefranc, (2001), Curr Protoc Immunol Appendix 1: Appendix 10).

Certain binding molecules of the invention preferably have a K_{D} for the ALWGPDPAAA (SEQ ID NO: 1)-HLA-A*02 complex of greater than (i.e. stronger than) a corresponding native TCR (also referred to as a non-mutated, or scaffold TCR). A higher affinity refers to a lower numerical value for K_{D} and indicates stronger binding. The K_{D} may be, for example, in the range of 1 pM to 50 μM. Binding molecules of the invention may have a K_{D} for the target complex of from about (i.e. +/−10%) 1 pM to about 400 nM, from about 1 pM to about 1000 pM, from about 1 pM to about 500 pM or from about 1 pM to about 100 pM. Said binding molecules may additionally, or alternatively, have a binding half-life (T_{1/2}) for the complex in the range of from about 0.5 min to about 50 h, from about 20 min to about 30 h, or from about 20 min to about 25 h. Preferably, binding molecules of the invention have a K_{D} for the ALWGPDPAAA (SEQ ID NO: 1)-HLA-A*02 complex of from about 1 pM to about 100 μM and/or a binding half-life from about 5 h to about 25 h. Such high-affinity is preferable for binding molecules in soluble format when associated with therapeutic agents and/or detectable labels. The affinity of a binding molecule may be measured at 25° C. Alternatively the affinity may be measured at 37° C. Methods for determining affinity of binding molecules are described herein.

Binding molecules of the invention comprising native TCR variable domains may have a K_{D} for the complex of from about 1 μM to about 200 μM, or from about 1 μM to about 100 μM. Such binding molecules may be preferable for adoptive therapy applications.

Certain preferred mutated binding molecules have a binding affinity for, and/or a binding half-life for, the ALWGPDPAAA (SEQ ID NO: 1)-HLA-A*02 complex that is substantially higher than that of a corresponding native TCR. Increasing the binding affinity of a native TCR may reduce the specificity of the TCR for its peptide-MHC ligand; this is demonstrated in Zhao et al., (2007) J. Immunol, 179:9, 5845-5854. However, certain binding molecules of the invention surprisingly demonstrate a high level of specificity for the ALWGPDPAAA (SEQ ID NO: 1)-HLA-A*02 complex, despite having substantially higher binding affinity than the native TCR.

The binding molecules of the invention preferably have the property of specifically binding to the ALWGPDPAAA (SEQ ID NO: 1)-HLA-A*02 complex. As used herein, “specific” binding refers to a binding molecule that binds to the ALWGPDPAAA (SEQ ID NO: 1)-HLA-A*02 complex with higher affinity than to other peptide-HLA complexes. Highly specific binding molecules of the invention are particularly suitable for therapeutic use due to the reduced risk of off-target effects. Specificity in the context of binding molecules of the invention can be determined according to their ability to recognise target cells that are antigen positive, whilst having minimal ability to recognise target cells that are antigen negative.

Specificity may be determined by assessing the ability of a binding molecule to bind to the ALWGPDPAAA (SEQ ID NO: 1)-HLA-A*02 complex with higher affinity than to a panel of alternative peptide-HLA complexes. This may, for example, be determined by the Surface Plasmon Resonance (SPR) method described herein e.g., in Example 1. Said panel may contain at least 2, at least 3, at least 5, or at least 10, alternative peptide-HLA complexes. The alternative peptides may share a low or high level of sequence identity with SEQ ID NO: 1 and may be naturally presented in vivo. Alternative peptides are preferably derived from commonly expressed proteins and or proteins expressed in healthy human tissues. Suitable alternative peptides with high sequence similarity to ALWGPDPAAA (SEQ ID NO: 1), include the “mimetics” described in Example 1 and provided in SEQ ID NOs: 67 and 88 to 91. Binding of the binding molecule to the ALWGPDPAAA (SEQ ID NO: 1)-HLA-A*02 complex may be at least 2 fold greater than to other naturally presented peptide HLA complexes, more preferably at least 10 fold, or at least 50 fold or at least 100 fold greater, even more preferably at least 1000 fold greater. Naturally occurring variants of the ALWGPDPAAA (SEQ ID NO: 1) peptide may be excluded from the definition of alternative peptide-HLA complexes.

An alternative or additional approach to determine binding molecule specificity may be to identify the peptide recognition motif of the binding molecule using sequential mutagenesis, e.g. alanine/serine scanning, of the target peptide, as described in Example 1. Residues that form part of the binding motif are those that are not permissible to substitution. Non-permissible substitutions may be defined as those peptide positions in which the binding affinity of the binding molecule is reduced by at least 50%, or preferably at least 80% relative to the binding affinity for the non-mutated peptide. Such an approach is further described in Cameron et al., (2013), Sci Transl Med. 2013 Aug. 7; 5 (197): 197ra103 and WO2014096803. Binding molecule specificity in this case may be determined by identifying alternative motif containing peptides, particularly alternative motif containing peptides in the human proteome, and testing these peptides for binding to the binding molecule. Binding of the binding molecule to one or more alternative peptides may indicate a lack of specificity. In this case further testing of binding molecule specificity via cellular assays may be required. A low tolerance for (alanine/serine) substitutions in the central part of the peptide indicate that the TCR has a high specificity and therefore presents a low risk for cross-reactivity with alternative peptides.

A binding molecule having the property of binding to the ALWGPDPAAA (SEQ ID NO: 1)-HLA-A*02 complex may bind to this complex with higher affinity relative to another peptide-HLA-A*02 complex. The binding molecule may bind to the ALWGPDPAAA (SEQ ID NO: 1)-HLA-A*02 complex with an affinity which is at least two-fold, at least three-fold, at least four-fold, at least five-fold, at least 6-fold, at least 10-fold, at least 100-fold, at least 500-fold, or at least 1000-fold higher than its affinity for a ALLGPDPAAA (SEQ ID NO: 67)-HLA-A*02 complex. The difference in affinity between the ALWGPDPAAA (SEQ ID NO: 1)-HLA-A*02 complex and the ALLGPDPAAA (SEQ ID NO: 67)-HLA-A*02 complex may be referred to as the “affinity window”. Notably, ALLGPDPAAA (SEQ ID NO: 67) differs from ALWGPDPAAA (SEQ ID NO: 1) at only a single amino acid position. As demonstrated in Example 4, the present inventors have identified binding molecules that have an affinity for the ALWGPDPAAA (SEQ ID NO: 1)-HLA-A*02 complex in the low picomolar range and with an affinity window (relative to SEQ ID NO: 67) of at least 500-fold. Such high affinity and high specificity binding molecules are suitable for use as a soluble therapeutic agent, for example.

Certain binding molecules of the invention are able to bind in vitro to antigen positive cells, in particular those cells presenting low levels of antigen (i.e. in the order of 5-100) and generate a highly potent anti-inflammatory response, such as CD8+ cell killing and/or CD4+ inflammation inhibition. Such binding molecules may be in soluble form and linked to an immune suppressor such as a PD-1 agonist or an interleukin or cytokine such as IL-2, IL-4, IL-10 or IL-13. The anti-inflammatory response that is measured may be CD8+ cell killing and/or CD4+ inflammation inhibition, and or inhibition of CD8⁺ T cell signalling pathways. Suitable methods for assessing an anti-inflammatory response will be known in the art and include the Jurkat NFAT cell reporter assay described in Example 4. Preferably a highly potent response is one with IC₅₀ value in the pM range, i.e. 1000 pM or lower. Preferably the maximum inhibition obtained in reporter assays is greater than 50%, for example 80% or more.

The term “mutation” encompasses designed substitutions, insertions and deletions (e.g., engineered or designed substitutions, insertions and deletions) and is used synonymously with “modification”. A “mutation” refers to a difference in amino acid sequence and does not necessarily require the act of replacing one amino acid with another. Mutations to a native (also referred to as parental, natural, unmutated, wild type, or scaffold) binding molecule may confer beneficial therapeutic properties, such as higher affinity, higher stability, higher specificity and/or high potency. For example, mutations may include those that increase the binding affinity (k_{D}) and/or binding half life (T1/2) of the binding molecule to the ALWGPDPAAA (SEQ ID NO: 1)-HLA-A*02 complex.

The term “stability” in the context of the present invention refers to physical and chemical stability and can be evaluated qualitatively and/or quantitatively using various analytical techniques that are described in the art and are reviewed in for example Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Such methods include the evaluation of aggregate formation (for example using size exclusion chromatography (SEC)), by measuring turbidity (for example by dynamic light scattering (DLS) or light obscuration (LO)) and/or by visual inspection (for example by determining colour and clarity). Stability may be assessed under stress conditions, such as high temperatures e.g. 37° C., or multiple freeze thaw cycles (such as 3, 4 or 5, or more cycles).

In the binding molecules of the invention, there may be at least one mutation in the TCR alpha chain variable region. There may be one, two, three, four, five, six, seven, eight, nine, ten, or more, mutations in the alpha chain CDRs (i.e. in total across all three CDRs). For example, there may be four mutations in the alpha chain CDRs. There may be three mutations in the alpha chain CDR1 and/or one mutation in the alpha chain CDR2 and/or one mutations in the alpha chain CDR3.

In the binding molecule of the invention, the mutations in the alpha chain CDRs may be conservative, semi-conservative, tolerated or other phenotypically silent mutations, as described herein. Other suitable conservative, semi-conservative, tolerated or other phenotypically silent mutations will be apparent to those skilled in the art.

The mutations in the alpha chain CDRs may be selected from K28R (CDR1), H29G (CDR1), G32S (CDR1) and Q53N (CDR2), numbered according to SEQ ID NO: 26. Thus, there may be any or all of these mutations, optionally in combination with other mutations.

A mutated alpha chain variable domain may be paired with any beta chain variable domain defined herein.

Mutations in the beta chain CDRs may be conservative, semi-conservative, tolerated or other phenotypically silent mutations, as described herein. Other suitable conservative, semi-conservative, tolerated or other phenotypically silent mutations will be apparent to those skilled in the art.

There may be at least one mutation in the TCR beta chain variable region of the binding molecules of the invention. There may be one, two, three, four, five, six, seven, eight, nine, ten or more, mutations in the beta chain CDRs (i.e. in total across all three CDRs). For example, there may be six or seven mutations in the beta chain CDRs. There may be one, two or three mutations in the beta chain CDR1 and/or two mutations in the beta chain CDR2 and/or one mutation in the beta chain CDR3.

The mutations in the beta chain CDRs may be selected from L27M (CDR1), Q28N (CDR1), S30N (CDR1), V52A (CDR2), F54I (CDR2) and A104S (CDR3), numbered according to SEQ ID NO: 74. A mutated beta chain variable domain may be paired with any alpha chain variable region defined herein.

Mutation(s) within the CDRs, relative to a native sequence, may improve the binding affinity or stability of the binding molecule of the invention but may additionally or alternatively confer other advantages such as improved specificity or improved potency when fused to an immune effector. Mutations may also reduce the risk of destabilising post-translational modifications, such as deamidation. Mutations at one or more positions may additionally or alternatively affect the interaction of an adjacent position with the cognate pMHC complex, for example by providing a more favourable angle for interaction. Mutations may include those that result in a reduction in non-specific binding, i.e. a reduction in binding to alternative antigens relative to the ALWGPDPAAA (SEQ ID NO: 1)-HLA-A*02 complex. Mutations may include those that increase efficacy of folding and/or stability and/or manufacturability. Some mutations may contribute to each of these characteristics; others may contribute to affinity but not to specificity, for example, or to specificity but not to affinity, or to stability but not affinity, etc.

The binding molecule of the present invention may comprise one of the following combinations of TCR alpha chain variable domain CDRs and TCR beta chain variable domain CDRs:
(a) alpha chain CDR1, CDR2 and CDR3 amino acid sequences of DRGSQS (SEQ ID NO: 5), IYSNGD (SEQ ID NO: 6) and AVRGNEKLT (SEQ ID NO: 7), respectively, and beta chain CDR1, CDR2 and CDR3 amino acid sequences of MNHNY (SEQ ID NO: 15), SVGAGI (SEQ ID NO: 16) and ASSYMTGELF (SEQ ID NO: 17), respectively;
(b) alpha chain CDR1, CDR2 and CDR3 amino acid sequences of DKHSQG (SEQ ID NO: 23), IYSNGD (SEQ ID NO: 6) and AVRGNEKLT (SEQ ID NO: 7), respectively, and beta chain CDR1, CDR2 and CDR3 amino acid sequences of MNHSY (SEQ ID NO: 28), SVGVGF (SEQ ID NO: 29) and ASAYMTGELF (SEQ ID NO: 30), respectively;
(c) alpha chain CDR1, CDR2 and CDR3 amino acid sequences of DKHSQG (SEQ ID NO: 23), IYSNGD (SEQ ID NO: 6) and AVRGNEKLT (SEQ ID NO: 7), respectively, and beta chain CDR1, CDR2 and CDR3 amino acid sequences of MQHSY (SEQ ID NO: 32), SVGVGF (SEQ ID NO: 29) and ASAYMTGELF (SEQ ID NO: 30), respectively; or
(d) alpha chain CDR1, CDR2 and CDR3 amino acid sequences of DKHSQG (SEQ ID NO: 23), IYSQGD (SEQ ID NO: 27) and AVRGNEKLT (SEQ ID NO: 7), respectively, and beta chain CDR1, CDR2 and CDR3 amino acid sequences of LQHSY (SEQ ID NO: 35), SVGVGF (SEQ ID NO: 29) and ASAYMTGELF (SEQ ID NO: 30), respectively.

Preferably, the binding molecule comprises alpha chain CDR1, CDR2 and CDR3 amino acid sequences of DKHSQG (SEQ ID NO: 23), IYSQGD (SEQ ID NO: 27) and AVRGNEKLT (SEQ ID NO: 7), respectively, and beta chain CDR1, CDR2 and CDR3 amino acid sequences of LQHSY (SEQ ID NO: 35), SVGVGF (SEQ ID NO: 29) and ASAYMTGELF (SEQ ID NO: 30), respectively. These are the CDR sequences present in the TCRs referred to as “a19b19”, “a19b20”, “a19b21” and “a19b22” in the Examples.

Mutations may additionally, or alternatively, be made outside the CDRs, within the framework regions; such mutations may result in improved therapeutic properties, such as improve affinity, and/or specificity, and/or stability, and/or the yield of a purified soluble form of the binding molecule. For example, the binding molecules of the invention may, additionally or alternatively, comprise one or more mutations at the N terminus of FR1 (the first, N-terminal framework region), of one of both of the alpha and beta chain variable domains, relative to the canonical framework sequences for a given TRAV and TRBV chain. Such mutations may improve the efficiency of N-terminal methionine cleavage. The removal of an N-terminal initiation methionine is often crucial for the function and stability of proteins. Inefficient cleavage may be detrimental for a therapeutic, since it may result in a heterogeneous protein product, and or the presence of the initiation methionine may be immunogenic in humans. In some cases an initiation methionine may be present in the binding molecules of the invention.

In the binding molecule of the invention, the alpha chain variable domain framework regions may comprise the following sequences:
FR1—AKEVEQNSGPLSVPEGAIASLQCTYS (SEQ ID NO: 25), optionally with one, two or three mutations therein,
FR2—FFWYRQYSGKSPELIMS (SEQ ID NO: 9), optionally with one, two or three mutations therein,
FR3—KEDGRFTAQLNKASQYVSLLIRDSQPSDSATYLC (SEQ ID NO: 10), optionally with one, two or three mutations therein,
FR4—FGTGTRLTIIP (SEQ ID NO: 11), optionally with one, two or three mutations therein, and/or the beta chain variable domain framework regions comprise the following sequences:
FR1—NAGVTQTPKFRILKIGQSMTLQCAQD (SEQ ID NO: 18), optionally with one, two or three mutations therein,
FR2—MYWYRQDPGMGLKPIYY (SEQ ID NO: 19), optionally with one, two or three mutations therein,
FR3—TDKGEVPQGYQVSRSTTEDFPLRLESAAPSQTSVYFC (SEQ ID NO: 75), optionally with one, two or three mutations therein,
FR4—FGEGSRLTVL (SEQ ID NO: 21), optionally with one, two or three mutations therein.

The alpha chain framework regions FR1, FR2, and FR3 may comprise amino acid sequences corresponding to a TRAV12-2*02 chain and/or the beta chain framework regions FR1, FR2 and FR3, may comprise amino acid sequences corresponding to those of a TRBV6-6*02 chain.

The FR4 region may comprise the joining region of the alpha and beta variable chains (TRAJ and TRBJ, respectively). The TRAJ region may comprise amino acid sequences corresponding to those of TRAJ48*01. The TRBJ region may comprise amino acid sequences corresponding to those of TRBJ2-2*01.

The alpha chain variable domain framework regions may have one, two, three, four, five or more mutations in total, relative to the above sequences. The alpha chain variable domain framework regions may have two mutations, relative to the above sequences. The mutation(s) in the TCR alpha chain variable domain framework regions may be selected from A1Q and Q22N, numbered according to SEQ ID NO: 26. The alpha chain variable domain framework regions may comprise no other mutations (other than those listed above).

The beta chain variable domain framework regions may have one, two, three, four, five or more mutations in total, relative to the above sequences. The beta chain variable domain framework regions may have one mutation, relative to the above sequences. The mutation(s) in the TCR beta chain variable domain framework regions may be selected from Q62N, Q62E, Q62D and Q65N, numbered according to SEQ ID NO: 74. The beta chain variable domain framework regions may comprise no other mutations relative to the above sequences.

The TCR alpha chain variable domain of the binding molecule of the invention may comprise respective framework amino acid sequences that have at least 90%, at least 91%, at least 92%, at least 93%, at least 94%, at least 95%, at least 96%, at least 97%, at least 98% or at least 99% identity to SEQ ID NOs: 25, 9, 10 and 11. The TCR beta chain variable domain of the binding molecule of the invention may comprise respective framework amino acid sequences that have at least 90%, at least 91%, at least 92%, at least 93%, at least 94%, at least 95%, at least 96%, at least 97%, at least 98% or at least 99% identity to SEQ ID NOs: 18, 19, 75 and 21. Alternatively, the stated percentage identity may be over the framework sequences when considered as a whole.

The TCR alpha chain variable domain may comprise any one of the amino acid sequences of SEQ ID NOs: 3, 22, 24 or 26, or an amino acid sequence having at least 80%, at least 90%, at least 95% or at least 98% identity to any one of SEQ ID NOs: 3, 22, 24, or 26. The TCR beta chain variable domain may comprise any one of the amino acid sequences of SEQ ID NOs: 13, 68, 31, 34, 74, 76 or 78, or an amino acid sequence having at least 80%, at least 90%, at least 95% or at least 98% identity to any one of SEQ ID NOs: 13, 68, 31, 34, 74, 76 or 78. As all alpha chain variable domains and beta chain variable domains are derived from the same scaffold TCR sequences (i.e., SEQ ID NO: 2 and SEQ ID NO: 12 respectively), it is expected that all alpha chain variable domain sequences are compatible with all beta chain variable domain sequences. Thus, the alpha chain variable domain may comprise an amino acid sequence provided in any one of SEQ ID NOS: 3, 22, 24, or 26, or an amino acid sequence with at least 90% identity thereto, and the beta chain variable domain may comprise an amino acid sequence provided in any one of SEQ ID NOs: 13, 68, 31, 34, 74, 76 or 78, or an amino acid sequence with at least 90% identity thereto.

The binding molecule may comprise one of the following combinations of alpha and beta chain variable domains:
(a) a TCR alpha chain variable domain comprising the amino acid sequence provided in SEQ ID NO: 22 and a TCR beta chain variable domain comprising the amino acid sequence provided in SEQ ID NO: 68;
(b) a TCR alpha chain variable domain comprising the amino acid sequence provided in SEQ ID NO: 24 and a TCR beta chain variable domain comprising the amino acid sequence provided in SEQ ID NO: 31;
(c) a TCR alpha chain variable domain comprising the amino acid sequence provided in SEQ ID NO: 26 and a TCR beta chain variable domain comprising the amino acid sequence provided in SEQ ID NO: 34;
(d) a TCR alpha chain variable domain comprising the amino acid sequence provided in SEQ ID NO: 26 and a TCR beta chain variable domain comprising the amino acid sequence provided in SEQ ID NO: 74;
(e) a TCR alpha chain variable domain comprising the amino acid sequence provided in SEQ ID NO: 26 and a TCR beta chain variable domain comprising the amino acid sequence provided in SEQ ID NO: 76; or
(f) a TCR alpha chain variable domain comprising the amino acid sequence provided in SEQ ID NO: 26 and a TCR beta chain variable domain comprising the amino acid sequence provided in SEQ ID NO: 78.

Preferably, the alpha chain variable domain comprises the amino acid sequence of SEQ ID NO: 26 [a19] and the beta chain variable domain comprises the amino acid sequence of SEQ ID NO: 74 [b20]. In this regard, the invention provides a binding molecule comprising a peptide-major histocompatibility complex (pMHC)-binding domain having the property of binding to ALWGPDPAAA (SEQ ID NO: 1) in complex with HLA-A*02, wherein the pMHC-binding domain comprises (i) an alpha chain, comprising at least a TCR alpha chain variable domain, and (ii) a beta chain, comprising at least a TCR beta chain variable domain, wherein the TCR alpha chain variable domain comprises the amino acid sequence provided in SEQ ID NO: 26, or an amino acid sequence having at least 80%, at least 90%, at least 95% or at least 98% identity to SEQ ID NO: 26, and the TCR beta chain variable domain comprises the amino acid sequence provided in SEQ ID NO: 74, or an amino acid sequence having at least 80%, at least 90%, at least 95% or at least 98% identity to SEQ ID NO: 74.

In the binding molecules of the invention, the variable domains, and where present the constant domains and/or any other domains, may be organised in any suitable format/arrangement that allows antigen binding. As used herein, a “format” of a binding molecule specifies a defined spatial arrangement of domains, in particular variable and optionally constant domains. Characteristics of such protein formats are the number of polypeptide chains (single polypeptide chain, double polypeptide chain or multiple polypeptide chains), the type and length of linkers connecting different domains, the number of antigen binding moieties (and thus the number of valences), the number of different antigen binding moieties (and thus the number of specificities for different antigens, e.g. bispecific, multispecific), and the order and orientation of variable domains (e.g. cross-over, parallel). For example, the alpha chain and beta chain of the pMHC-binding domain may be arranged in a monoclonal TCR format, in which the two chains are linked by a disulphide bond, either within the constant domains or variable domains, or in which the variable domains are fused to one or more dimerization domains. Alternatively, the variable domains may be arranged in a single polypeptide chain format in the presence or absence of one or more constant domains, or the variable domains may be arranged in diabody format. Other suitable formats may be used.

The alpha chain and/or beta chain of the pMHC binding domain may comprise a TCR constant domain or fragment thereof, for example an alpha chain TRAC constant domain and/or a beta chain TRBC1 or TRBC2 constant domain. Thus, the alpha chain may comprise a TCR alpha chain constant domain and/or the beta chain may comprise a TCR beta chain constant domain. As will be appreciated by those skilled in the art the term TRAC and TRBC1/2 also encompasses natural polymorphic variants, for example N to K at position 4 of TRAC (Bragado et al International immunology. 1994 February; 6(2):223-30).

Where present, one or both of the constant domains may contain mutations, substitutions or deletions relative to native constant domain sequences. The constant domains may be truncated, i.e. having no transmembrane or cytoplasmic domains. Thus, the terms “TCR alpha chain constant domain” and “TCR beta chain constant domain” encompass such truncated amino acid sequences, provided that they retain a sufficient length derived from native TCR constant domains such that they promote association of the alpha chain and the beta chain. For example, the binding molecule of the invention may comprise the extracellular region of a TCR alpha chain constant domain and/or the extracellular region of a TCR beta chain constant domain. Alternatively, the constant domains may be full-length by which it is meant that extracellular, transmembrane and cytoplasmic domains are all present. The TRAC and TRBC domain sequences may be modified by truncation or substitution to delete the native disulphide bond between Cys4 of exon 2 of TRAC and Cys2 of exon 2 of TRBC1 or TRBC2. The alpha and/or beta chain constant domain sequence(s) may have an introduced disulphide bond between residues of the respective constant domains, as described, for example, in WO 03/020763. Thus, the binding molecule may comprise a non-native covalent disulphide bond that links a residue of the TCR alpha chain constant domain to a residue of the TCR beta chain constant domain. Preferably the alpha and beta constant domains may be modified by substitution of cysteine residues at position Thr 48 of TRAC and position Ser 57 of TRBC1 or TRBC2, the said cysteines forming a non-natural disulphide bond between the alpha and beta constant domains of the TCR. TRBC1 or TRBC2 may additionally include a cysteine to alanine mutation at position 75 of the constant domain and an asparagine to aspartic acid mutation at position 89 of the constant domain. One or both of the extracellular constant domains, e.g., present in an αβ heterodimer, may be further truncated at the C terminus or C termini, for example by up to 15, or up to 10, or up to 8 or fewer amino acids. One or both of the extracellular constant domains, e.g., present in an αβ heterodimer, may be truncated at the C terminus or C termini by, for example, up to 15, or up to 10 or up to 8 amino acids. The C terminus of the alpha chain extracellular constant domain may be truncated by 8 amino acids.

A binding molecule of the invention may comprise the extracellular region of a TCR alpha chain constant domain, optionally truncated at the C terminus by up to 15 amino acids, and/or the extracellular region of a TCR beta chain constant domain, optionally truncated at the C terminus by up to 15 amino acids.

The TCR alpha chain constant domain may comprise the amino acid sequence provided in SEQ ID NO: 37, or an amino acid sequence that has at least 90% identity, such as at least 90%, at least 91%, at least 92%, at least 93%, at least 94%, at least 95%, at least 96%, at least 97%, at least 98%, or at least 99% identity, to the sequence provided in SEQ ID NO: 37, and/or the TCR beta chain constant domain may comprise the amino acid sequence provided in SEQ ID NO: 39, or an amino acid sequence that has at least 90% identity, such as at least 90%, at least 91%, at least 92%, at least 93%, at least 94%, at least 95%, at least 96%, at least 97%, at least 98%, or at least 99% identity, to the sequence provided in SEQ ID NO:39.

The pMHC-binding domain may comprise an alpha chain comprising a TCR alpha chain constant domain comprising the amino acid sequence provided in SEQ ID NO: 37 and a beta chain comprising a TCR beta chain constant domain comprising the amino acid sequence provided in SEQ ID NO: 39. The binding molecule may not comprise a transmembrane or cytoplasmic domain of a TCR.

Alternatively, rather than full-length or truncated constant domains, there may be no TCR constant domains. Accordingly, the pMHC-binding domain may consist of the TCR alpha and beta variable domains, optionally with additional domains as described herein. Additional domains include but are not limited to immune suppressor domains (such as antibody domains), Fc domains or albumin binding domains, therapeutic agents or detectable labels.

The binding molecule may comprise the alpha chain and beta chain in a single polypeptide chain format (i.e., the alpha and beta chains may be present in the same polypeptide chain). Single polypeptide chain formats include, but are not limited to, αβ TCR polypeptides of the Vα-L-Vβ, Vβ-L-Vα, Vα-Cα-L-Vβ, Vα-L-Vβ-Cβ, or Vα-Cα-L-Vβ-Cβ types, wherein Vα and Vβ are TCR α and β variable regions respectively, Cα and Cβ are TCR α and β constant regions respectively, and L is a linker sequence (Weidanz et al., (1998) J Immunol Methods. December 1; 221(1-2):59-76; Epel et al., (2002), Cancer Immunol Immunother. November; 51(10):565-73; WO 2004/033685; WO9918129).

The term “linker” as used herein refers to one or more amino acid residues inserted between domains, or a domain and an agent, to provide sufficient mobility for the domains or elements, for example the domains of the binding molecules of the invention to fold correctly to form the antigen binding sites. A linker may be inserted at the transition between variable domains or between variable domains and constant domains (or other domains), respectively, at the amino acid sequence level. The transition between domains can be identified because the approximate size of antibody domains as well as TCR domains is well understood by those skilled in the art. The precise location of a domain transition can be determined by locating peptide stretches that do not form secondary structural elements such as beta-sheets or alpha-helices as demonstrated by experimental data or as can be assumed by techniques of modeling or secondary structure prediction.

Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length, The linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used in binding molecules of the invention include, but are not limited to: GGGGS (SEQ ID NO: 73), GGGSG (SEQ ID NO: 55), GGSGG (SEQ ID NO: 56), GSGGG (SEQ ID NO: 57), GSGGGP (SEQ ID NO: 58), GGEPS (SEQ ID NO: 59), GGEGGGP (SEQ ID NO: 60), GGEGGGSEGGGS (SEQ ID NO: 61), GGGSGGGG (SEQ ID NO: 62), GGGGSGGGGSGGGGSGGGGSGGGS (SEQ ID NO: 63), GGGGGGGGSGGGGSGGGGS (SEQ ID NO: 64), EAAAK (SEQ ID NO: 65) and EAAAKEAAAKEAAAK (SEQ ID NO: 66).

Single polypeptide chain TCRs may be soluble, i.e., they do not comprise a transmembrane or cytoplasmic domain. Single polypeptide chain TCRs may have an introduced disulphide bond between residues of the respective constant domains, as described in WO 2004/033685. Single polypeptide chain TCRs are further described in WO2004/033685; WO98/39482; WO01/62908; Weidanz et al. (1998) J Immunol Methods 221(1-2): 59-76; Hoo et al. (1992) Proc Natl Acad Sci U S A 89(10):4759-4763; Schodin (1996) Mol Immunol 33(9): 819-829).

Alternatively the binding molecule may comprise two or more polypeptide chains, wherein the alpha chain and the beta chain are comprised in separate polypeptide chains.

The TCR variable domains may be arranged in diabody format. In the diabody format two single polypeptide chain fragments dimerize in a head-to-tail orientation resulting in a compact molecule with a molecular mass similar to tandem scFv (~50 kDa).

Particularly suitable alpha chain sequences include, but are not limited to, any one of SEQ ID NOS: 2, 70, 36, and 40. Particularly suitable beta chain sequences include, but are not limited to, any one of SEQ ID NOs: 12, 69, 38, 41, 80, 81 and 82. Such sequences do not comprise transmembrane or cytoplasmic domains. It is expected that every alpha chain sequence (i.e., SEQ ID NOs: 2, 70, 36, and 40) is compatible with every beta chain sequence (i.e., SEQ ID NOs: 12, 69, 38, 41, 80, 81 and 82), as they are all derived from the same native (scaffold) TCR sequences (SEQ ID NOs: 2 and 12 respectively). Therefore, the alpha chain may comprise an amino acid sequence provided in any one of SEQ ID NOs: 2, 70, 36, and 40, or an amino acid sequence that has at least 90% identity, such as at least 90%, at least 91%, at least 92%, at least 93%, at least 94%, at least 95%, at least 96%, at least 97%, at least 98%, or at least 99%, to the amino acid sequence provided in any one of SEQ ID NOs: 2, 70, 36, and 40, and the beta chain may comprise an amino acid sequence provided in any one of SEQ ID NOs: 12, 69, 38, 41, 80, 81 and 82, or an amino acid sequence that has at least 90% identity, such as at least 90%, at least 91%, at least 92%, at least 93%, at least 94%, at least 95%, at least 96%, at least 97%, at least 98%, or at least 99%, to the amino acid sequence provided in any one of SEQ ID NOs: 12, 69, 38, 41, 80, 81 and 82.

More specifically, the pMHC-binding domain may comprise
(a) an alpha chain comprising the amino acid sequence of SEQ ID NO: 2 and a beta chain comprising the amino acid sequence of SEQ ID NO: 12;
(b) an alpha chain comprising the amino acid sequence of SEQ ID NO: 70 and a beta chain comprising the amino acid sequence of SEQ ID NO: 69;
(c) an alpha chain comprising the amino acid sequence of SEQ ID NO: 36 and a beta chain comprising the amino acid sequence of SEQ ID NO: 38;
(d) an alpha chain comprising the amino acid sequence of SEQ ID NO: 40 and a beta chain comprising the amino acid sequence of SEQ ID NO: 41;
(e) an alpha chain comprising the amino acid sequence of SEQ ID NO: 40 and a beta chain comprising the amino acid sequence of SEQ ID NO: 80;
(f) an alpha chain comprising the amino acid sequence of SEQ ID NO: 40 and a beta chain comprising the amino acid sequence of SEQ ID NO: 81; or
(g) an alpha chain comprising the amino acid sequence of SEQ ID NO: 40 and a beta chain comprising the amino acid sequence of SEQ ID NO: 82.

Preferably, the pMHC-binding domain comprises an alpha chain comprising the amino acid sequence of SEQ ID NO: 40 and a beta chain comprising the amino acid sequence of SEQ ID NO: 80. In this regard, the invention provides a binding molecule comprising a pMHC-binding domain having the property of binding to ALWGPDPAAA (SEQ ID NO: 1) in complex with HLA-A*02, wherein the pMHC-binding domain comprises an alpha chain comprising the amino acid sequence of SEQ ID NO: 40, or an amino acid sequence that has at least 90% identity, such as at least 90%, at least 91%, at least 92%, at least 93%, at least 94%, at least 95%, at least 96%, at least 97%, at least 98%, or at least 99%, to the amino acid sequence of SEQ ID NO: 40, and a beta chain comprising the amino acid sequence of SEQ ID NO: 80, or an amino acid sequence that has at least 90% identity, such as at least 90%, at least 91%, at least 92%, at least 93%, at least 94%, at least 95%, at least 96%, at least 97%, at least 98%, or at least 99%, to the amino acid sequence of SEQ ID NO: 80.

Binding molecules of the invention are useful for delivering detectable labels or therapeutic agents to antigen presenting cells and tissues containing antigen presenting cells. They may therefore comprise or be associated (covalently or otherwise) with a detectable label (for diagnostic purposes wherein the binding molecule is used to detect the presence of cells presenting the cognate antigen); and/or a therapeutic agent, including immune effectors; and or a pharmacokinetics (PK) modifying moiety.

Examples of PK modifying moieties include, but are not limited to, PEG (Dozier et al., (2015) Int J Mol Sci. October 28; 16(10):25831-64 and Jevsevar et al., (2010) Biotechnol J. January; 5(1):113-28), PASylation (Schlapschy et al., (2013) Protein Eng Des Sel. August; 26(8):489-501), albumin, and albumin binding domains, (Dennis et al., (2002) J Biol Chem. September 20; 277(38):35035-43), and/or unstructured polypeptides (Schellenberger et al., (2009) Nat Biotechnol. December; 27(12):1186-90). Further PK modifying moieties include immunoglobulin Fc domains. PK modifying moieties may serve to extend the in vivo half-life of binding molecules of the invention. Thus, a PK modifying moiety may be a half-life extending domain.

The binding molecule may comprise a half-life extending domain. A “half-life extending domain”, as used herein, refers to a protein domain for extending the half-life of the binding molecule of the invention, relative to a corresponding binding molecule lacking the half-life extending domain. For example, the half-life extending domain may comprise an immunoglobulin Fc domain. The immunoglobulin Fc domain may be any antibody Fc region. Thus, the binding molecule of the invention may comprise an Fc region, such as an IgG Fc region. The Fc region is the tail region of an antibody that interacts with cell surface Fc receptors and some proteins of the complement system. The Fc region typically comprises two polypeptide chains, i.e., two “portions” FC1 and FC2, both having two or three heavy chain constant domains (termed CH2, CH3 and CH4), and a hinge region. The two portions may be linked by one or more disulphide bonds within the hinge region. Fc regions from immunoglobulin subclasses IgG1, IgG2 and IgG4 bind to and undergo FcRn mediated recycling, thus extending the half-life of the binding molecule. The interaction of IgG with FcRn has been localized in the Fc region covering parts of the CH2 and CH3 domain. Particularly suitable immunoglobulin Fc for use in the present invention include but are not limited to Fc domains from IgG1 or IgG4. For example, the Fc region may be an IgG1 Fc region, i.e., the FC1 and FC2 regions may be IgG1 Fc sequences. The Fc region may be derived from human sequences, for example a wild-type human IgG1 Fc region (SEQ ID NO: 92). In this regard, the FC1 and FC2 region may each comprise, or consist of, an amino acid sequence that is at least 90%, at least 95% or at least 98% identical to SEQ ID NO: 92.

The two portions of the Fc region may comprise mutations relative to a wild-type or unmodified Fc sequence. Mutations include substitutions, insertions and deletions. Such mutations may be made for the purpose of introducing desirable therapeutic properties. For example, to facilitate hetero-dimerisation, knobs into holes (KiH) mutations may be engineered into the CH3 domain. Thus, the half-life extending domain may comprise one or more amino acid substitutions which facilitate dimerisation of the FC1 region and the FC2 region. Such substitutions include “Knob-in-hole” substitutions. In this case, one chain (i.e. one of the FC1 or FC2 regions) is engineered to contain a bulky protruding residue (i.e. the knob), such as Y, and the other chain (i.e., the other of the FC1 and FC2 regions) is engineered to contain a complementary pocket (i.e. the hole). For example, a knob may be constructed by replacing a small amino acid side chain with a larger side chain. A hole may be constructed by replacing a large amino acid side chain with a smaller side chain. Without wishing to be bound to theory, this is thought to stabilize a hetero-dimer of the FC1 and FC2 regions by favouring formation of the hetero-dimer over other species, for example homomultimers of FC1 and FC2, thereby enhancing the stability and manufacturability of the binding molecule of the invention.

Suitable positions and substitutions for KiH mutations, and other mutations for facilitating dimerisation of Fc regions, are known in the art and include those described in Merchant et al., Nat Biotechnol 16:677 (1998) and Ridgway et al., Prot Engineering 9:617 (1996) and Atwell et al. J Mol Biol 270,1 (1997): 26-35. For example, the substitutions forming corresponding knobs and holes in two Fc regions may correspond to one or more pairs provided in the following table:

| CH3 of one of the FC1 | CH3 of the other of |
|---|---|
| and FC2 regions | the FC1 and FC2 regions |
| T366Y | Y407T |
| T366W | Y407A |
| T366W | T366S:L368A:Y407V |
| F405A | T394W |
| Y407T | T366Y |
| T366Y:F405A | T394W:Y407T |
| T366W:F405W | T394S:Y407A |
| F405W:Y407A | T366W:T394S |
| F405W | T394S |

The substitutions in the table above are denoted by the original residue, followed by the position using the EU numbering system, and then the import residue (all residues are given in single-letter amino acid code). Multiple substitutions are separated by a colon.

The FC1 and FC2 regions may comprise one or more substitutions in the table above. For example:
(i) one of the FC1 region and the FC2 region may comprise one or more amino acid substitutions selected from the group consisting of T366S, L368A, T394S, F405A, Y407A, Y407T and Y407V, according to the EU numbering scheme; and
(ii) the other of the FC1 region and the FC2 region may comprise one or more amino acid substitutions selected from the group consisting of T366W, T366Y, T366W, T394W and F405W according to the EU numbering scheme. The substitutions in (i) and (ii) are hole-forming and knob-forming substitutions respectively. The FC1 region may comprise one or more of the substitutions in (i) and the FC2 region may comprise one or more of the substitutions in (ii).

For example:
(i) one of the FC1 region and the FC2 region may comprise one or more amino acid substitutions selected from the group consisting of T366S, L368A, and Y407V, according to the EU numbering scheme; and
(ii) the other of the FC1 region and the FC2 region may comprise a T366W amino acid substitution, according to the EU numbering scheme. The FC1 region may comprise one or more of the substitutions in (i) and the FC2 region may comprise the substitution in (ii).

Preferably, (i) one of the FC1 region and the FC2 region comprises T366S, L368A, and Y407V amino acid substitutions, according to the EU numbering scheme; and (ii) the other of the FC1 region and the FC2 region comprises a T366W amino acid substitution, according to the EU numbering scheme. For example, the FC1 region may comprise T366S, L368A, and Y407V amino acid substitutions, according to the EU numbering scheme; and the FC2 region may comprise a T366W amino acid substitution, according to the EU numbering scheme.

The Fc region may also comprise one or more mutations that attenuate an effector function of the Fc region. Exemplary effector functions include, without limitation, complement-dependent cytotoxicity (CDC) and/or antibody-dependent cellular cytotoxicity (ADCC). The modification to attenuate effector function may be a modification that alters the glycosylation pattern of the Fc region, e.g., a modification that results in an aglycosylated Fc region. Alternatively, the modification to attenuate effector function may be a modification that does not alter the glycosylation pattern of the Fc region. The modification to attenuate effector function may reduce or eliminate binding to human effector cells, binding to one or more Fc receptors, and/or binding to cells expressing an Fc receptor. FC1 and/or FC2 may comprise one or more amino acid substitutions which prevent or reduce binding to FcγR. For example, FC1 and/or FC2 may comprise one or more amino acid substitutions selected from the group consisting of S228P, E233P, L234A, L235A, L235E, L235P, G236R, G237A, P238S, F241A, V264A D265A, H268A, D270A, N297A, N297G, N297Q, E318A, K322A, L328R, P329G, P329A, A330S, A330L, P331A and P331S, according to the EU numbering scheme. Particular modifications include a N297G or N297A substitution in the Fc region of human IgG1 (EU numbering). Other suitable modifications include L234A, L235A and P329G substitutions in the Fc region of human IgG1 (EU numbering), that result in attenuated effector function. The FC1 and/or FC2 region may comprise a substitution at residue N297, numbering according to EU index. For example, the substitution may be an N297G or N297A substitution. Other suitable mutations (e.g., at residue N297) are known to those skilled in the art.

Fc regions having reduced effector function refers to variants that reduce effector function (e.g., CDC, ADCC, and/or binding to FcR, etc. activities) by at least 10%, 20%, 30%, 40%, 50%, 60%, 70%, 80%, 90%, 95%, 97%, 98%, 99% or more as compared to the effector function achieved by a wild-type Fc region (e.g., an Fc region not having a mutation to reduce effector function, although it may have other mutations). The Fc variants having reduced effector function may be Fc variants that eliminate all detectable effector function as compared to a wild-type Fc region. Assays for measuring effector function are known in the art and described below.

In vitro and/or in vivo cytotoxicity assays can be conducted to confirm the reduction/depletion of CDC and/or ADCC activities. For example, Fc receptor (FcR) binding assays can be conducted to ensure that the Fc region or fusion protein lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)).

Substitutions may be introduced into the FC1 and FC2 regions that abrogate or reduce binding to Fcy receptors and/or increase binding to FcRn, and/or prevent Fab arm exchange, and/or remove protease sites. In this regard, the half-life extending domain may also comprise one or more amino acid substitutions which prevent or reduce binding to activating receptors. The half-life extending domain may comprise one or more amino acid substitutions which prevent or reduce binding to FcγR. For example, the FC1 region and/or the FC2 region may comprise a N297G amino acid substitution, according to the EU numbering scheme. Both the FC1 region and the FC2 region may comprise the N297G amino acid substitution.

The serum half-life of binding molecules comprising Fc regions may be further increased by increasing the binding affinity of the Fc region for FcRn, and thus the half-life extending domain may comprise one or more modifications (e.g., amino acid substitutions, amino acid insertions, or amino acid deletions) which promote binding to FcRn. The one or more modifications are relative to a corresponding wild-type Fc region (e.g., a human IgG1 or IgG4 Fc region). Methods of measuring binding to FcRn are known. Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody substitutions with improved or diminished binding to FcRs. In particular, Mackness et al., MAbs. 11:1276-1288 (2019) describes suitable amino acid substitutions in antibody Fc regions for enhancing binding to FcRn.

The term “half-life”, as used herein, refers to a pharmacokinetic property of a binding molecule that is a measure of the mean survival time of binding molecules following their administration to a subject. Binding molecule half-life can be expressed as the time required to eliminate 50 percent of a known quantity of a binding molecule from the subject's body (or other mammal) or a specific compartment thereof, for example, as measured in serum, i.e., circulating half-life, or in other tissues.

An increase in half-life allows for the reduction in amount of drug given to a patient as well as reducing the frequency of administration. An increase in half-life can also be beneficial, for example, for treatment of an autoimmune disease or condition. Binding proteins with increased half-lives may also be generated by modifying amino acid residues identified as involved in the interaction between the Fc and the FcRn receptor. Binding proteins comprising Fc regions that comprise one or more modifications which promote binding to FcRn may have an increased half-life of about 5%, about 10%, about 15%, about 20%, about 25%, about 30%, about 35%, about 40%, about 45%, about 50%, about 60%, about 65%, about 70%, about 80%, about 85%, about 90%, about 95%, about 100%, about 125%, about 150% or more as compared to a binding protein comprising a native Fc region. Binding proteins comprising Fc regions that comprise one or more modifications which promote binding to FcRn may have an increased half-life of about 2 fold, about 3 fold, about 4 fold, about 5 fold, about 10 fold, about 20 fold, about 50 fold or more, or is between 2 fold and 10 fold, or between 5 fold and 25 fold, or between 15 fold and 50 fold, as compared to binding proteins comprising a native Fc region.

The modification(s) (e.g., amino acid substitutions, amino acid insertions, or amino acid deletions) in the Fc region which promote binding to FcRn may be at one or more positions selected from the group consisting of 234, 235, 236, 237, 238, 239, 240, 241, 243, 244, 245, 247, 251, 252, 254, 255, 256, 262, 263, 264, 265, 266, 267, 268, 269, 279, 280, 284, 292, 296, 297, 298, 299, 305, 313, 316, 325, 326, 327, 328, 329, 330, 331, 332, 333, 334, 339, 341, 343, 370, 373, 378, 392, 416, 419, 421, 440 and 443 as numbered by the EU index as set forth in Kabat. Optionally, the Fc region may comprise a non-naturally occurring amino acid residue at additional and/or alternative positions known in the art.

More specifically, the Fc region can comprise at least one substitution selected from the group consisting of 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235F, 236E, 239D, 239E, 239N, 239Q, 239F, 239T, 239H, 239Y, 2401, 240A, 240T, 240M, 241W, 241L, 241Y, 241E, 241R. 243W, 243L 243Y, 243R, 243Q, 244H, 245A, 247L, 247V, 247G, 251F, 252Y, 254T, 255L, 256E, 256M, 2621, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 2651, 265L, 265H, 265T, 2661, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 2961, 296H, 269G, 297S, 297D, 297E, 298H, 298I, 298T, 298F, 2991, 299L, 299A, 299S, 299V, 299H, 299F, 299E, 3051, 313F, 316D, 325Q, 325L, 3251, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 339T, 370E, 370N, 378D, 392T, 396L, 416G, 419H, 421K, 440Y and 434W as numbered by the EU index as set forth in Kabat. Optionally, the Fc region may comprise additional and/or alternative non-naturally occurring amino acid residues known in the art.

The modification(s) (e.g., amino acid substitutions, amino acid insertions, or amino acid deletions) in the Fc region which promote binding to FcRn may be at one or more positions selected from the group consisting of 234, 235 and 331, as numbered by the EU index as set forth in Kabat. For example, the Fc region may comprise at least one substitution selected from the group consisting of 234F, 235F, 235Y, and 331S, as numbered by the EU index as set forth in Kabat.

The modification(s) (e.g., amino acid substitutions, amino acid insertions, or amino acid deletions) in the Fc region which promote binding to FcRn may be at one or more positions selected from the group consisting of 239, 330 and 332, as numbered by the EU index as set forth in Kabat. For example, the Fc region may comprise at least one substitution selected from the group consisting of 239D, 330L and 332E, as numbered by the EU index as set forth in Kabat.

The modification(s) (e.g., amino acid substitutions, amino acid insertions, or amino acid deletions) in the Fc region which promote binding to FcRn may be at one or more positions selected from the group consisting of 252, 254, and 256, as numbered by the EU index as set forth in Kabat. For example, the Fc region may comprise at least one substitution selected from the group consisting of 252Y, 254T and 256E, as numbered by the EU index as set forth in Kabat, as described in U.S. Pat. No. 7,083,784, the contents of which are herein incorporated by reference in its entirety. The Fc region may comprise all of the following substitutions: 252Y, 254T and 256E, as numbered by the EU index as set forth in Kabat.

The substitutions which promote binding to FcRn listed above are relative to a corresponding wild-type Fc region (e.g., a human IgG1 or IgG4 Fc region) and may be present in one of or preferably both of the FC1 and FC2 portions of the Fc region. In other words, the substitutions refer to amino acids that are not normally present in a corresponding wild-type Fc region, for example a human IgG1 or IgG4 Fc region. In this regard, a “substitution”, as used herein, refers to the presence of one of the listed amino acids in a polypeptide and does not necessarily require replacing one amino acid with another.

Additionally or alternatively, mutations may be made for manufacturing reasons, for example to remove or replace amino acids that may be subject to post-translational modifications such as glycosylation, as described herein.

The two Fc regions FC1 and FC2 may both comprise a CH2 and CH3 constant domain and all or part of a hinge sequence. The hinge sequence may correspond substantially or partially to a hinge region from IgG1, IgG2, IgG3 or IgG4. The hinge sequence may be an IgG1 hinge sequence, such as the amino acid sequence provided in SEQ ID NO: 50 or 95. The hinge may alternatively be an IgG4 hinge sequence, such as the amino acid sequence provided in SEQ ID NO: 96. A preferred IgG hinge sequence is SEQ ID NO: 50. The hinge may comprise all or part of a core hinge domain and all or part of a lower hinge region.

As mentioned above, a binding molecule of the invention may comprise a half-life extending domain comprising a first portion of an IgG Fc region (FC1) and a second portion of an IgG1 Fc region (FC2). FC1 and FC2 dimerize to form the Fc region. The FC1 region may comprise, or consist of, an amino acid sequence that is at least 80% identical to the sequence of SEQ ID NO: 49 or 93 and the FC2 region may comprise, or consist of, an amino acid sequence that is at least 80% identical to the sequence of SEQ ID NO: 52 or 94. The FC1 region may comprise, or consist of, an amino acid sequence that is at least 90%, at least 95%, or at least 98% identical to the sequence of SEQ ID NO: 49 or 93 and the FC2 region may comprise, or consist of, an amino acid sequence that is at least 90%, at least 95%, or at least 98% identical to the sequence of SEQ ID NO: 52 or 94. the FC1 region may comprise, or consist of, the amino acid sequence provided in SEQ ID NO: 49 and the FC2 region may comprise, or consist of, the amino acid sequence provided in SEQ ID NO: 52. Alternatively, the FC1 region may comprise, or consist of, the amino acid sequence provided in SEQ ID NO: 93 and the FC2 region may comprise, or consist of, the amino acid sequence provided in SEQ ID NO: 94.

As the skilled person would appreciate, the sequences provided above for FC1 and FC2 are suitable vice versa. For example, (a) either FC1 or FC2 may comprise the amino acid sequence provided in SEQ ID NO: 49, or an amino acid sequence that has at least 90%, at least 95%, or at least 98% identity to the amino acid sequence provided in SEQ ID NO: 49, and (b) the other of FC1 and FC2 may comprise the amino acid sequence provided in SEQ ID NO: 52, or an amino acid sequence that has at least 90%, at least 95%, or at least 98% identity to the amino acid sequence provided in SEQ ID NO: 52. Similarly, (a) either FC1 or FC2 may comprise the amino acid sequence provided in SEQ ID NO: 93, or an amino acid sequence that has at least 90%, at least 95%, or at least 98% identity to the amino acid sequence provided in SEQ ID NO: 93, and (b) the other of FC1 and FC2 may comprise the amino acid sequence provided in SEQ ID NO: 94, or an amino acid sequence that has at least 90%, at least 95%, or at least 98% identity to the amino acid sequence provided in SEQ ID NO: 94.

The Fc region may be fused to the other domains (e.g., alpha or beta chain) in the binding molecule of the invention via a linker, and/or a hinge sequence in any suitable orientation. Alternatively no linker may be used. Preferred formats for binding molecules comprising an Fc region are described herein below.

Alternatively, the half-life extending domain may be albumin or an albumin-binding domain. As is known in the art, albumin has a long circulatory half-life of 19 days, due in part to its size, being above the renal threshold, and by its specific interaction and recycling via FcRn. Attachment to albumin is a well-known strategy to improve the circulatory half-life of a therapeutic molecule in vivo. Albumin may be attached non-covalently, through the use of a specific albumin binding domain, or covalently, by conjugation or direct genetic fusion. Examples of therapeutic molecules that have exploited attachment to albumin for improved half-life are given in Sleep et al., Biochim Biophys Acta. 2013 December; 1830 (12): 5526-34.

The albumin-binding domain may be any moiety capable of binding to albumin, including any known albumin-binding moiety. Albumin binding domains may be selected from endogenous or exogenous ligands, small organic molecules, fatty acids, peptides and proteins that specifically bind albumin. Examples of preferred albumin binding domains include short peptides, such as described in Dennis et al., J Biol Chem. 2002 Sep. 20; 277(38):35035-43 (for example the peptide QRLMEDICLPRWGCLWEDDF); proteins engineered to bind albumin such as antibodies, antibody fragments and antibody like scaffolds, for example Albudab® (O'Connor-Semmes et al., Clin Pharmacol Ther. 2014 December; 96 (6): 704-12), commercially provided by GSK and Nanobody® (Van Roy et al., Arthritis Res Ther. 2015 May 20; 17:135), commercially provided by Ablynx; and proteins based on albumin binding domains found in nature such as Streptococcal protein G Protein (Stork et al., Eng Des Sel. 2007 November; 20 (11): 569-76), for example Albumod® commercially provided by Affibody. Preferably, albumin is human serum albumin (HSA). The affinity of the albumin binding domain for human albumin may be in the range of picomolar to micromolar. Given the extremely high concentration of albumin in human serum (35-50 mg/ml, approximately 0.6 mM), it is calculated that substantially all of the albumin binding domains will be bound to albumin in vivo.

The albumin-binding moiety may be fused to the C or N terminus of the other domains (i.e., the TCR variable domains and/or TCR constant domains and/or an immune effector domain), in any suitable order or configuration. The albumin-binding moiety may be fused to one or more of the other domains (i.e., the TCR variable domains and/or TCR constant domains and/or an immune effector domain) via a linker. Suitable linkers are known in the art and include those described herein. Where the albumin-binding moiety is linked to the TCR, it may be linked to either the alpha or beta chains, with or without a linker.

Detectable labels for diagnostic purposes include for instance, fluorescent labels, radiolabels, enzymes, nucleic acid probes and contrast reagents.

For some purposes, the binding molecules of the invention may be aggregated into a complex comprising several binding molecules to form a multivalent binding molecule complex. There are a number of human proteins that contain a multimerisation domain that may be used in the production of multivalent binding molecule complexes. For example the tetramerisation domain of p53 which has been utilised to produce tetramers of scFv antibody fragments which exhibited increased serum persistence and significantly reduced off-rate compared to the monomeric scFv fragment (Willuda et al. (2001) J. Biol. Chem. 276 (17) 14385-14392). Haemoglobin also has a tetramerisation domain that could be used for this kind of application. A multivalent binding molecule complex of the invention may have enhanced binding capability for the complex compared to a non-multimeric native (also referred to as parental, natural, unmutated wild type, or scaffold) T cell receptor heterodimer of the invention. Thus, multivalent complexes of binding molecules of the invention are also included within the invention. Such multivalent binding molecule complexes according to the invention are particularly useful for tracking or targeting cells presenting particular antigens in vitro or in vivo, and are also useful as intermediates for the production of further multivalent binding molecule complexes having such uses.

Therapeutic agents which may be associated with or comprised in the binding molecules of the invention include immune suppressors, such as interleukins, cytokines or immune checkpoint agonists. To ensure that the therapeutic effects are exercised in the desired location the agent could be inside a liposome or other nanoparticle structure linked to the binding molecule so that the compound is released slowly. This will prevent damaging effects during the transport in the body and ensure that the agent has maximum effect after binding of the binding molecule to the relevant antigen presenting cells.

Examples of suitable therapeutic agents include, but are not limited to, antibodies, or fragments thereof, including immune checkpoint agonist antibodies (e.g. anti-PD-1) or alternative protein scaffolds with antibody-like binding characteristics (e.g. DARPins). Other suitable therapeutic agents include ligands of immune checkpoint receptors, interleukins or cytokines. IL-2, IL-4, IL-10 and IL-13 are example cytokines suitable for association with the binding molecules of the present invention.

Binding molecules of the invention may be multispecific. As used herein, the term “multispecific” refers to a binding molecule comprising two or more binding moieties, including the pMHC-binding domain. Such binding molecules are able to bind to the ALWGPDPAAA (SEQ ID NO: 1)-HLA-A*02 complex and a further one or more different targets. For example, the binding molecule may be bispecific. Such binding molecules comprise a pMHC-binding domain that binds to the ALWGPDPAAA (SEQ ID NO: 1)-HLA-A*02 complex and one other binding moiety (e.g., an antibody antigen binding moiety) that binds to a different target.

Binding molecules of the invention may comprise an immune suppressor. As used herein, the term “immune suppressor” refers to any molecule, e.g., a protein, that is capable of inhibiting an immune response, such as inhibiting T cell activation. The immune suppressor may bind to a target (e.g. antigen). For example, the immune suppressor may be an immune checkpoint agonist, i.e., a molecule that induces immune checkpoint signalling. Such immune suppressors are described below.

The immune suppressor may comprise an antigen-binding moiety that is capable of binding to an antigen. The antigen of the immune suppressor may be located on an immune cell, such as a T cell. The binding molecule may comprise an antibody or antigen binding fragment thereof. The term “antibody” as used herein is meant to include conventional/native antibodies and engineered antibodies, in particular functional antibody fragments, single chain antibodies, single domain antibodies, bispecific or multispecific antibodies. “Native” or “conventional” refers to an antibody that has the same type of domains and domain arrangements as an antibody found in nature and comprises antibody-derived CDR and FR sequences. In a native/conventional antibody, two heavy chains are linked to each other by disulfide bonds and each heavy chain is linked to a light chain by a disulfide bond. The variable domains of both light (VL) and heavy (VH) chains determine binding recognition and specificity to the antigen. Conventional antibody binding sites are made up of residues that are primarily from the “antibody complementarity determining regions” (CDRs) or hypervariable regions. Occasionally, residues from non-hypervariable or framework regions (FR) influence the overall domain structure and hence the binding site. CDRs refer to amino acid sequences that together define the binding affinity and specificity of the natural Fv region of a native antibody binding site. The light and heavy chains of a conventional antibody each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a VH and VL.

“Engineered” antibody formats include functional antibody fragments, single chain antibodies, single domain antibodies, and chimeric, humanized, bispecific or multispecific antibodies. Engineered antibody formats further include constructs in which TCR-derived CDRs, possibly including additional 3, 2 or 1N and/or C terminal framework residues, or entire TCR-derived variable domains are grafted onto antibody heavy or light chains. A “functional antibody fragment” refers to a portion of a full-length antibody, or a protein that resembles a portion of a full-length antibody, that retains the ability to bind to its target antigen, in particular the antigen binding region or variable region of the full-length antibody. Examples of functional antibody “fragments” include Fv, Fab, F(ab′)2, Fab′, dsFv, (dsFv)2, scFv, sc(Fv)2 and diabodies. For example, a binding molecule of the invention may comprise a scFv. A functional antibody fragment may also be a single domain antibody, such as a heavy chain antibody. In this regard, the term “single domain antibody” refers to an antibody that consists of a single antibody variable domain (e.g., a heavy chain variable domain). Thus, the immune suppressor may comprise a VHH (i.e., the variable domain of a heavy chain antibody), for example. As is known in the art, the antigen binding site of a single domain antibody, such as a VHH, may comprise three CDRs (as opposed to six in a conventional four-chain antibody). The term “antigen binding moiety of an antibody”, as used herein, encompasses such binding sites. Alternatively, or additionally, the binding molecule may comprise a Fab or Fv fragment. The term “Fab” (“fragment antigen-binding”) denotes an antigen-binding fragment of an antibody, which comprises the antibody light chain (VL-CL) and the variable and CH1 domain (VH-CH1) of the antibody heavy chain. Fab fragments typically have a molecular weight of about 50,000 Dalton. The Fv fragment is the N-terminal part of the Fab fragment of an antibody and consists of the variable portions of one light chain (VL) and one heavy chain (VH).

The immune suppressor may comprise an antibody heavy chain variable region (VH) and an antibody light chain variable region (VL), which associate to form the antigen-binding moiety that is capable of binding to the antigen. Thus, the antigen binding moiety may comprise the VH and the VL. For example, the immune suppressor may comprise a scFv comprising the VH and VL.

Other suitable antigen binding moieties are heavy chain antibodies (hcAb), single domain antibodies (sdAb), minibodies (Tramontano et al (1994) J. Mol. Recognition 7, 9-24), the variable domain of camelid heavy chain antibodies (VHH), the variable domain of the new antigen receptors (VNAR), affibodies (Nygren P. A. (2008) FEBS J. 275, 2668-2676), alphabodies (see WO2010066740), designed ankyrin-repeat domains (DARPins) (Stumpp et al (2008) Drug Discovery Today 13, 695-701), anticalins (Skerra et al (2008) FEBS J. 275, 2677-2683), knottins (Kolmar et al (2008) FEBS J. 275, 2684-2690) and engineered CH2 domains (nanoantibodies, see Dimitrov D S (2009) mAbs 1, 26-28).

The antigen binding moiety may be, or comprise, a heavy chain variable domain that comprises, consists or essentially consists of four framework regions (FR1 to FR4 respectively) and three complementarity determining regions (CDR1 to CDR3 respectively); or any suitable fragment of such a heavy chain variable domain (which retains the antigen binding site). The antigen binding moiety may be a heavy chain antibody. The antigen binding moiety may be a heavy chain variable domain sequence of an antibody that is derived from a conventional four-chain antibody, such as, without limitation, a VH sequence that is derived from a human antibody. Preferably, the antigen binding moiety is, or comprises, the variable domain of a heavy chain antibody (e.g., a camelid antibody), such as a VHH (also referred to herein as a “VHH domain”). Preferably, the antigen binding moiety is a VHH.

As described herein, the immune suppressor may comprise an antigen binding moiety (e.g., an antibody antigen binding moiety) that binds to an antigen located on an immune cell. In the context of the present invention, “immune cell” may refer to, for example, a T cell or a B cell. In particular, the antigen of the antigen-binding moiety may be a T cell surface antigen.

The target (i.e., antigen) of the immune suppressor may be an immune modulator. For example, the target may be an immune checkpoint molecule, such as PD-1 (Programmed Death 1 receptor), A2AR (Adenosine A2A receptor), A2BR (Adenosine A2B receptor), B7-H3 (B7 Homolog 3, also called CD276) B7-H4 (B7 Homolog 4, also called VTCN1), BTLA (B and T Lymphocyte Attenuator, also called CD272), CTLA-4 (Cytotoxic T-Lymphocyte-Associated protein 4, also called CD152), IDO (Indoleamine 2,3-dioxygenase), CD200 Receptor, KIR (Killer-cell Immunoglobulin-like Receptor), TIGIT (T cell Immunoreceptor with Ig and ITIM domains), LAG3 (Lymphocyte Activation Gene-3), NOX2 (nicotinamide adenine dinucleotide phosphate NADPH oxidase isoform 2), TIM-3 (T-cell Immunoglobulin domain and Mucin domain 3), VISTA (V-domain Ig suppressor of T cell activation), SIGLEC7 (Sialic acid-binding immunoglobulin-type lectin 7, also called CD328), and SIGLEC9 (Sialic acid-binding immunoglobulin-type lectin 9, also called CD329). In this regard, the immune suppressor may be an agonist of one or more of the above immune checkpoint molecules. Thus, the immune suppressor may be an immune checkpoint agonist (i.e., to inhibit immune activation). Suitable immune checkpoint agonists, including native ligands and antibodies, are reviewed in Paluch et al Front Immunol, 2018, 9:2306, for example.

Instead of comprising an antigen binding moiety of an antibody, the immune suppressor may comprise one of a receptor-ligand pair, whereby the immune suppressor is capable of binding to the other of the receptor-ligand pair. The target ligand or receptor may be located on an immune cell. For example, the immune suppressor may comprise a ligand of an immune checkpoint molecule described above. In particular, the immune suppressor may comprise the extracellular region of PD-L1 (Uniprot ref: Q9NZQ7) or PD-L2 (Q9BQ51) or a functional fragment thereof (i.e., a portion that is capable of binding to PD-1). For example, the immune suppressor may comprise the amino acid sequence provided in SEQ ID NO: 102, or an amino acid sequence having at least 90% or at least 95% identity to SEQ ID NO: 102. Such an immune suppressor may engage an immune cell by binding to PD-1 and stimulate PD-1 signalling.

Alternatively the immune suppressor may comprise an agonist antibody that binds to, and preferably stimulates signalling of, an immune checkpoint molecule. For example, the immune suppressor may be, or comprise, a PD-1 agonist antibody (e.g., single domain antibody). Preferably, such PD-1 agonists do not compete with PD-L1 for binding to PD-1. Preferably, such PD-1 agonists have a high degree of specificity for PD-1 and give rise to a potent inhibitory response when tested in reporter assays as described in Example 4. The PD-1 agonist may be a full-length antibody or fragment thereof, such as a scFv antibody or a Fab fragment, or a single domain antibody. Examples of such antibodies are provided in WO2011110621 and WO2010029434 and WO2018024237. Thus the antigen of the immune suppressor may be PD-1 and the antigen binding moiety of the immune suppressor may be a PD-1 agonist. The antigen binding moiety of the immune suppressor may comprise a single domain antibody, optionally a VHH. Preferably, the immune suppressor is a PD-1 agonist VHH.

As described above, the immune suppressor is preferably a PD-1 agonist. As used herein, the term “PD-1 agonist” refers to any molecule that is capable of binding to PD-1 and activating PD-1 signalling, including e.g., the PD-1 ligand, PD-L1, and PD-1 agonist antibodies. Activation of the PD-1 pathway down-regulates immune activity, promoting peripheral immune tolerance and preventing autoimmunity (Keir et al., Annu Rev Immunol, 26:677-704, 2008; Okazaki et al., Int Immunol 19:813-824, 2007). PD-1 is a transmembrane receptor protein expressed on the surface of activated immune cells, including T cells, B cells, NK cells and monocytes (Agata et al., Int Immunol 8:765-772, 1996). The cytoplasmic tail of PD-1 comprises an immunoreceptor tyrosine-based inhibitory motif (ITIM). PD-L1 and PD-L2 are the natural ligands of PD-1 and are expressed on the surface of antigen presenting cells (Dong et al., Nat Med., 5:1365-1369, 1999; Freeman et al., J Exp Med 192:1027-1034, 2000; Latchman et al., Nat Immunol 2:261-268, 2001). Upon ligand engagement, phosphatases are recruited to the ITIM region of PD-1 leading to inhibition of TCR-mediated signaling, and subsequent reduction in lymphocyte proliferation, cytokine secretion and cytotoxic activity. PD-1 may also induce apoptosis in T cells via its ability to inhibit survival signals from co-stimulation (Keir et al., Annu Rev Immunol, 26:677-704, 2008). Targeted activation of the PD-1 pathway therefore provides an approach for the treatment of autoimmune conditions, such as T1DM.

The antigen binding moiety of the immune suppressor may have the general structure:
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively. In this regard, the antigen binding moiety may be a single domain antibody that binds to PD-1 and comprises CDRs, CDR1, CDR2 and CDR3, having the following amino acid sequences:
CDR1—GFTFSSYA (SEQ ID NO: 43), optionally with one, two or three mutations therein,
CDR2—IASDGAST (SEQ ID NO: 44), optionally with one, two or three mutations therein, and
CDR3—CARGGYLTYDRY (SEQ ID NO: 45), optionally with one, two or three mutations therein. Preferably, CDR1 comprises the amino acid sequence provided in SEQ ID NO: 43, CDR2 comprises the amino acid sequence provided in SEQ ID NO: 44 and CDR3 comprises the amino acid sequence provided in SEQ ID NO: 45. Preferably, the antigen-binding moiety is a VHH.

The single domain antibody may be a VHH comprising the amino acid sequence of SEQ ID NO: 42, or a humanised version thereof, or an amino acid sequence having at least 80%, at least 90%, at least 95% or at least 98% identity to SEQ ID NO: 42. Alternatively, the single domain antibody may be a VHH comprising the amino acid sequence of SEQ ID NO: 71, or an amino acid sequence having at least 80%, at least 90%, at least 95% or at least 98% identity to SEQ ID NO: 71.

Preferably, the single domain antibody is a VHH comprising the amino acid sequence provided in SEQ ID NO: 71.

The immune suppressor, if present, may be covalently linked to the pMHC-binding domain via the C- or N-terminus of the alpha chain or beta chain, optionally via a linker sequence. For example, the C-terminus of the immune suppressor may be covalently linked to the N terminus of the beta chain, optionally via a linker sequence. Suitable linker sequences are known in the art. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length. For example, the immune suppressor may be covalently linked to the pMHC-binding domain via the C- or N-terminus of the alpha chain or beta chain via a linker sequence selected from GGGGS (SEQ ID NO: 73), GGGSG (SEQ ID NO: 55), GGSGG (SEQ ID NO: 56), GSGGG (SEQ ID NO: 57), GSGGGP (SEQ ID NO: 58), GGEPS (SEQ ID NO: 59), GGEGGGP (SEQ ID NO: 60), GGEGGGSEGGGS (SEQ ID NO: 61), GGGSGGGG (SEQ ID NO: 62), GGGGSGGGGSGGGGSGGGGSGGGS (SEQ ID NO: 63), GGGGSGGGGSGGGGSGGGGS (SEQ ID NO: 64), EAAAK (SEQ ID NO: 65) and EAAAKEAAAKEAAAK (SEQ ID NO: 66).

The immune suppressor may be covalently linked to the C- or N-terminus of the alpha chain or beta chain via a linker sequence of GGGGS (SEQ ID NO: 73). For example, the C-terminus of the immune suppressor may be covalently linked to the N terminus of the beta chain via a linker sequence of GGGGS (SEQ ID NO: 73).

A binding molecule comprising an immune suppressor, may comprise at least a first polypeptide chain and a second polypeptide chain. For example, the binding molecule may comprise:
(a) a first polypeptide chain comprising the immune suppressor and the beta chain of the pMHC binding domain; and
(b) a second polypeptide chain comprising the alpha chain of the pMHC-binding domain.
   The C-terminus of the immune suppressor may be covalently linked to the N-terminus of the beta chain, optionally via the linker sequence of SEQ ID NO: 73.

The first polypeptide may comprise the structure N—IS-Beta-C, and the second polypeptide may comprise the structure N-Alpha-C, where “IS” refers to the immune suppressor, “Beta” refers to the beta chain of the pMHC-binding domain and “Alpha” refers to alpha chain of the pMHC-binding domain. The immune suppressor may be as described herein above and may comprise an scFv or VHH, for example. The first and/or second polypeptide chain(s) may or may not further comprise other polypeptide sequences at the N- or C-terminus.

Exemplary binding molecules of the invention comprising two polypeptides as described immediately above include a2b3VHH (consisting of SEQ ID NOs: 70 and 72), a18b16VHH (consisting of SEQ ID NOs: 36 and 46), a19b19VHH (consisting of SEQ ID NOs: 40 and 47), a19b20VHH (consisting of SEQ ID NOs: 40 and 83), a19b21VHH (consisting of SEQ ID NOs: 40 and 84) and a19b22VHH (consisting of SEQ ID NOs: 40 and 85).

A binding molecule comprising two polypeptides as described above may comprise a first polypeptide chain comprising an amino acid sequence as set forth in any one of SEQ ID NOs: 72, 46, 47, 83, 84 and 85, or an amino acid sequence that has at least 90% identity, such as at least 90%, at least 91%, at least 92%, at least 93%, at least 94%, at least 95%, at least 96%, at least 97%, at least 98%, at least 99% or 100% identity, to an amino acid sequence as set forth in any one of SEQ ID NOs: 72, 46, 47, 83, 84 and 85, and
a second polypeptide chain comprising the amino acid sequence as set forth in any one of SEQ ID NOS: 70, 36 and 40, or an amino acid sequence that has at least 90% identity, such as at least 90%, at least 91%, at least 92%, at least 93%, at least 94%, at least 95%, at least 96%, at least 97%, at least 98%, at least 99% or 100% identity, to an amino acid sequence as set forth in any one of SEQ ID NOs: 70, 36 and 40.

More particularly, a binding molecule in the format described above may comprise
(a) a first polypeptide chain comprising the amino acid sequence of SEQ ID NO: 72 and a second polypeptide chain comprising the amino acid sequence of SEQ ID NO: 70;
(b) a first polypeptide chain comprising the amino acid sequence of SEQ ID NO: 46 and a second polypeptide chain comprising the amino acid sequence of SEQ ID NO: 36;
(c) a first polypeptide chain comprising the amino acid sequence of SEQ ID NO: 47 and a second polypeptide chain comprising the amino acid sequence of SEQ ID NO: 40;
(d) a first polypeptide chain comprising the amino acid sequence of SEQ ID NO: 83 and a second polypeptide chain comprising the amino acid sequence of SEQ ID NO: 40;
(e) a first polypeptide chain comprising the amino acid sequence of SEQ ID NO: 84 and a second polypeptide chain comprising the amino acid sequence of SEQ ID NO: 40; or
(f) a first polypeptide chain comprising the amino acid sequence of SEQ ID NO: 85 and a second polypeptide chain comprising the amino acid sequence of SEQ ID NO: 40.

Preferably, the binding molecule comprises a first polypeptide chain and second polypeptide chain, wherein the first polypeptide chain comprises the amino acid sequence set forth in SEQ ID NO: 83 and the second polypeptide chain comprises the amino acid sequence set forth in SEQ ID NO: 40. Thus, the invention provides a binding molecule having the property of binding to ALWGPDPAAA (SEQ ID NO: 1) in complex with HLA-A*02, wherein the binding molecule comprises a first polypeptide chain comprising a TCR beta chain covalently linked to a PD-1 agonist VHH and a second polypeptide chain comprising a TCR alpha chain, wherein the first polypeptide chain comprises the amino acid sequence set forth in SEQ ID NO: 83 and the second polypeptide chain comprises the amino acid sequence set forth in SEQ ID NO: 40.

The binding molecules comprising two polypeptide chains (i.e., a first and a second polypeptide chain) described above may further comprise another one or more polypeptide chains (e.g., a third polypeptide chain). Thus, the binding molecules of the invention may comprise three polypeptide chains, i.e., a first, a second and a third polypeptide chain. Such binding molecules include those that comprise an immunoglobulin Fc region, comprising a first portion FC1 and a second portion FC2, as described herein. For example, the binding molecule may comprise:
(a) a first polypeptide chain comprising the immune suppressor and either (i) the alpha chain or (ii) beta chain of the pMHC binding domain;
(b) a second polypeptide chain comprising FC1 and the other of (i) the alpha chain and (ii) the beta chain; and
(c) a third polypeptide chain comprising FC2.

The FC1 and FC2 regions may have any one or more of the features described herein above in relation to half-life extending domains.

The C-terminus of the immune suppressor may be covalently linked to the N-terminus of either (i) the alpha chain or (ii) beta chain of the pMHC binding domain, optionally via a linker sequence. The linker sequence may be SEQ ID NO: 73, for example. The C-terminus of the other of (i) the alpha chain and (ii) the beta chain may be covalently linked to the N-terminus of FC1. For example, the C-terminus of the other of (i) the alpha chain and (ii) the beta chain may be covalently linked to the N-terminus of FC1 via an IgG hinge sequence. The third polypeptide may comprise an IgG hinge sequence at the N-terminus of FC2. The IgG hinge may comprise the amino acid sequence of SEQ ID NO: 50.

The first polypeptide chain may comprise the beta chain of the pMHC binding domain and the second polypeptide chain may comprise the alpha chain of the pMHC binding domain. Thus, the C-terminus of the immune suppressor may be covalently linked to the N-terminus of the beta chain of the pMHC binding domain, optionally via a linker sequence such as SEQ ID NO: 73. The C-terminus of the alpha chain may be covalently linked to the N-terminus of FC1. For example, the C-terminus of the alpha chain may be covalently linked to the N-terminus of FC1 via an IgG hinge sequence, such as SEQ ID NO: 50.

The inventors have identified that molecules in the above “three-chain” format have the highest activity (i.e., potency and selectivity) of over 20 different formats tested. In this context, “three-chain” is used to describe a binding molecule that is expressed as three separate polypeptide chains which associate with each other to form a single three-dimensional folded structure comprising i) the pMHC-binding domain (formed by dimerisation of the alpha and beta chain), ii) the immune suppressor (e.g., VHH) and iii) the half-life extending domain comprising a Fc region. Preferably, for binding molecules in the format described immediately above, the first polypeptide chain comprises the beta chain of the pMHC-binding domain, and the second polypeptide chain comprises the alpha chain of the pMHC-binding domain. Such molecules have the general structure of
First polypeptide chain: N—IS-Beta-C;
Second polypeptide chain: N-Alpha-FC1-C; and
Third polypeptide chain: N-FC2-C,
   or, more specifically, the general structure of:
First polypeptide chain: N—IS-linker-Beta-C;
Second polypeptide chain: N-Alpha-hinge-FC1-C; and
Third polypeptide chain: N-hinge-FC2-C.

Where “N” and “C” are the N- and C-termini of each polypeptide chain respectively, “IS” is the immune suppressor, “Alpha” is the alpha chain of the pMHC-binding domain, “Beta” is the beta chain of the pMHC-binding domain, “linker” is a linker sequence as described herein (preferably SEQ ID NO: 73), and “hinge” is an IgG hinge sequence as described herein (SEQ ID NO: 50). The alpha and beta chains in the context of the three-chain binding molecules described above preferably do not comprise a transmembrane or cytoplasmic region.

Suitable linker sequences are described herein above. For example, if present, the linker (i.e., the linker in the first and/or second polypeptide chain) may have an amino acid sequence selected from the group of GGGGS (SEQ ID NO: 73), GGGSG (SEQ ID NO: 55), GGSGG (SEQ ID NO: 56), GSGGG (SEQ ID NO: 57), GSGGGP (SEQ ID NO: 58), GGEPS (SEQ ID NO: 59), GGEGGGP (SEQ ID NO: 60), GGEGGGSEGGGS (SEQ ID NO: 61), GGGSGGGG (SEQ ID NO: 62), GGGGSGGGGSGGGGSGGGGSGGGS (SEQ ID NO: 63), GGGGSGGGGSGGGGSGGGGS (SEQ ID NO: 64), EAAAK (SEQ ID NO: 65) and EAAAKEAAAKEAAAK (SEQ ID NO: 66). Preferably, the C-terminus of the immune suppressor is linked to the N-terminus of the beta chain in the first polypeptide chain via a linker having the amino acid sequence of GGGGS (SEQ ID NO: 73).

Suitable IgG hinge sequences are described herein above. For example, if present, the hinge (e.g., the hinge in the first and/or second and/or third polypeptide chain) may have the amino sequence provided in SEQ ID NO: 50, or an amino acid sequence that is at least 80%, at least 90%, or at least 95% identical to SEQ ID NO: 50. Preferably, the C-terminus of the alpha chain is linked to the N-terminus of FC1 in the second polypeptide chain via an IgG hinge having the amino acid sequence of SEQ ID NO: 50. Also, preferably, an IgG hinge having the amino acid sequence of SEQ ID NO: 50 may be present at the N-terminus of the third polypeptide chain (i.e., at the N-terminus of FC2).

A binding molecule of the invention may comprise a first polypeptide chain, second polypeptide chain and a third polypeptide chain, wherein
a) the first polypeptide comprises, from N- to C-terminus, a VHH (preferably a PD1 agonist VHH), a linker sequence (preferably SEQ ID NO: 73), and the beta chain of the pMHC-binding domain,
b) the second polypeptide comprises, from N- to C-terminus, the alpha chain of the pMHC-binding domain, an IgG hinge (preferably SEQ ID NO: 50), and a first portion of an Fc region (FC1), and
c) the third polypeptide comprises, from N- to C-terminus, an IgG hinge (preferably SEQ ID NO: 50) and a second portion of the Fc region (FC2).

The first polypeptide chain may comprise an amino acid sequence as set forth in any one of SEQ ID NOs: 72, 46, 47, 83, 84 and 85 or an amino acid sequence that has at least 90% identity, such as at least 90%, at least 91%, at least 92%, at least 93%, at least 94%, at least 95%, at least 96%, at least 97%, at least 98%, or at least 99%, to an amino acid sequence as set forth in any one of SEQ ID NOs: 72, 46, 47, 83, 84 and 85; and/or
the second polypeptide chain may comprise an amino acid sequence as set forth in any one of SEQ ID NOs: 48, 53, 54, 97, 99 or 100, or an amino acid sequence that has at least 90% identity, such as at least 90%, at least 91%, at least 92%, at least 93%, at least 94%, at least 95%, at least 96%, at least 97%, at least 98%, or at least 99%, to an amino acid sequence as set forth in any one of SEQ ID NOs: 48, 53, 54, 97, 99 or 100; and/or
the third polypeptide chain may comprise the amino acid sequence of SEQ ID NO: 51 or 98, or an amino acid sequence that has at least 90% identity, such as at least 90%, at least 91%, at least 92%, at least 93%, at least 94%, at least 95%, at least 96%, at least 97%, at least 98%, or at least 99%, to the amino acid sequence of SEQ ID NO: 51 or 98.

More particularly, the binding molecule may comprise
(a) a first polypeptide chain comprising the amino acid sequence of SEQ ID NO: 72, a second polypeptide chain comprising the amino acid sequence of SEQ ID NO: 48 and a third polypeptide chain comprising the amino acid sequence of SEQ ID NO: 51;
(b) a first polypeptide chain comprising the amino acid sequence of SEQ ID NO: 46, a second polypeptide chain comprising the amino acid sequence of SEQ ID NO: 53 and a third polypeptide chain comprising the amino acid sequence of SEQ ID NO: 51;
(c) a first polypeptide chain comprising the amino acid sequence of SEQ ID NO: 47, a second polypeptide chain comprising the amino acid sequence of SEQ ID NO: 54 and a third polypeptide chain comprising the amino acid sequence of SEQ ID NO: 51;
(d) a first polypeptide chain comprising the amino acid sequence of SEQ ID NO: 83, a second polypeptide chain comprising the amino acid sequence of SEQ ID NO: 54 and a third polypeptide chain comprising the amino acid sequence of SEQ ID NO: 51;
(e) a first polypeptide chain comprising the amino acid sequence of SEQ ID NO: 84, a second polypeptide chain comprising the amino acid sequence of SEQ ID NO: 54 and a third polypeptide chain comprising the amino acid sequence of SEQ ID NO: 51;
(f) a first polypeptide chain comprising the amino acid sequence of SEQ ID NO: 85, a second polypeptide chain comprising the amino acid sequence of SEQ ID NO: 54 and a third polypeptide chain comprising the amino acid sequence of SEQ ID NO: 51;
(g) a first polypeptide chain comprising the amino acid sequence of SEQ ID NO: 72, a second polypeptide chain comprising the amino acid sequence of SEQ ID NO: 97 and a third polypeptide chain comprising the amino acid sequence of SEQ ID NO: 98;
(h) a first polypeptide chain comprising the amino acid sequence of SEQ ID NO: 46, a second polypeptide chain comprising the amino acid sequence of SEQ ID NO: 99 and a third polypeptide chain comprising the amino acid sequence of SEQ ID NO: 98;
(i) a first polypeptide chain comprising the amino acid sequence of SEQ ID NO: 47, a second polypeptide chain comprising the amino acid sequence of SEQ ID NO: 100 and a third polypeptide chain comprising the amino acid sequence of SEQ ID NO: 98;
(j) a first polypeptide chain comprising the amino acid sequence of SEQ ID NO: 83, a second polypeptide chain comprising the amino acid sequence of SEQ ID NO: 100 and a third polypeptide chain comprising the amino acid sequence of SEQ ID NO: 98;
(k) a first polypeptide chain comprising the amino acid sequence of SEQ ID NO: 84, a second polypeptide chain comprising the amino acid sequence of SEQ ID NO: 100 and a third polypeptide chain comprising the amino acid sequence of SEQ ID NO: 98; or
(l) a first polypeptide chain comprising the amino acid sequence of SEQ ID NO: 85, a second polypeptide chain comprising the amino acid sequence of SEQ ID NO: 100 and a third polypeptide chain comprising the amino acid sequence of SEQ ID NO: 98.

For example, the first, second and third polypeptide chains may comprise the following amino acid sequences, respectively:
i) SEQ ID NO: 72, SEQ ID NO: 48 and SEQ ID NO: 51;
ii) SEQ ID NO: 46, SEQ ID NO: 53 and SEQ ID NO: 51;
iii) SEQ ID NO: 47; SEQ ID NO: 54 and SEQ ID NO: 51;
iv) SEQ ID NO: 83; SEQ ID NO: 54 and SEQ ID NO: 51;
v) SEQ ID NO: 84; SEQ ID NO: 54 and SEQ ID NO: 51;
vi) SEQ ID NO: 85; SEQ ID NO: 54 and SEQ ID NO: 51;
vii) SEQ ID NO: 72; SEQ ID NO: 97 and SEQ ID NO: 98;
viii) SEQ ID NO: 46; SEQ ID NO: 99 and SEQ ID NO: 98;
ix) SEQ ID NO: 47; SEQ ID NO: 100 and SEQ ID NO: 98;
x) SEQ ID NO: 83; SEQ ID NO: 100 and SEQ ID NO: 98;
xi) SEQ ID NO: 84; SEQ ID NO: 100 and SEQ ID NO: 98; or
xii) SEQ ID NO: 85; SEQ ID NO: 100 and SEQ ID NO: 98,
or sequences having at least 90%, at least 91%, at least 92%, at least 93%, at least 94%, at least 95%, at least 96%, at least 97%, at least 98%, or at least 99% identity thereto.
   The binding molecule may comprise
a) a first polypeptide chain comprising the amino acid sequence of SEQ ID NO: 83, or an amino acid sequence having at least 90%, at least 91%, at least 92%, at least 93%, at least 94%, at least 95%, at least 96%, at least 97%, at least 98%, or at least 99% identity to SEQ ID NO: 83;
b) second polypeptide chain comprising the amino acid sequence of SEQ ID NO: 54, or an amino acid sequence having at least 90%, at least 91%, at least 92%, at least 93%, at least 94%, at least 95%, at least 96%, at least 97%, at least 98%, or at least 99% identity to SEQ ID NO: 54; and
c) a third polypeptide chain comprising the amino acid sequence of SEQ ID NO: 51, or an amino acid sequence having at least 90%, at least 91%, at least 92%, at least 93%, at least 94%, at least 95%, at least 96%, at least 97%, at least 98%, or at least 99% identity to SEQ ID NO: 51.
   The binding molecule may comprise
a) a first polypeptide chain comprising the amino acid sequence of SEQ ID NO: 83, or an amino acid sequence having at least 90%, at least 91%, at least 92%, at least 93%, at least 94%, at least 95%, at least 96%, at least 97%, at least 98%, or at least 99% identity to SEQ ID NO: 83;
b) second polypeptide chain comprising the amino acid sequence of SEQ ID NO: 100, or an amino acid sequence having at least 90%, at least 91%, at least 92%, at least 93%, at least 94%, at least 95%, at least 96%, at least 97%, at least 98%, or at least 99% identity to SEQ ID NO: 100; and
c) a third polypeptide chain comprising the amino acid sequence of SEQ ID NO: 98, or an amino acid sequence having at least 90%, at least 91%, at least 92%, at least 93%, at least 94%, at least 95%, at least 96%, at least 97%, at least 98%, or at least 99% identity to SEQ ID NO: 98.
   The binding molecule may comprise
a) a first polypeptide chain having the following amino acid sequence:

(SEQ ID NO: 83)
AVQLVESGGG LVQPGGSLRL SCAASGFTFS SYAMTWVRQA PGKGPEWVSA IASDGASTSY
ADSVKGRFTI SRDNSKNTLY LQMNSLRPED TAVYYCARGG YLTYDRYGQG TLVTVSSGGG
GSNAGVTQTP KFRILKIGQS MTLQCAQDLQ HSYMYWYRQD PGMGLKPIYY SVGVGFTDKG
EVPQGYQVSR STTEDFPLRL ESAAPSQTSV YFCASAYMTG ELFFGEGSRL TVLEDLKNVF
PPEVAVFEPS EAEISHTQKA TLVCLATGFY PDHVELSWWV NGKEVHSGVC TDPQPLKEQP
ALQDSRYALS SRLRVSATFW QDPRNHFRCQ VQFYGLSEND EWTQDRAKPV TQIVSAEAWG
RAD;

b) a second polypeptide chain having the following amino acid sequence:

(SEQ ID NO: 54)
AKEVEQNSGP LSVPEGAIAS LQCTYSDKHS QGFFWYRQYS GKSPELIMSI YSQGDKEDGR
FTAQLNKASQ YVSLLIRDSQ PSDSATYLCA VRGNEKLTFG TGTRLTIIPN IQNPDPAVYQ
LRDSKSSDKS VCLFTDFDSQ TQVSQSKDSD VYITDKCVLD MRSMDFKSNS AVAWSQKSDF
ACANAFQNSI IPEDTDKTHT CPPCPAPELL GGPSVFLFPP KPKDTLMISR TPEVTCVVVD
VSHEDPEVKF NWYVDGVEVH NAKTKPREEQ YGSTYRVVSV LTVLHQDWLN GKEYKCKVSN
KALPAPIEKT ISKAKGQPRE PQVYTLPPSR DELTKNQVSL SCAVKGFYPS DIAVEWESNG
QPENNYKTTP PVLDSDGSFF LVSKLTVDKS RWQQGNVFSC SVMHEALHNH YTQKSLSLSP
GK;

and
c) a third polypeptide chain having the following amino acid sequence:

(SEQ ID NO: 51)
DKTHTCPPCP APELLGGPSV FLFPPKPKDT LMISRTPEVT CVVVDVSHED PEVKFNWYVD
GVEVHNAKTK PREEQYGSTY RVVSVLTVLH QDWLNGKEYK CKVSNKALPA PIEKTISKAK
GQPREPQVYT LPPSRDELTK NQVSLWCLVK GFYPSDIAVE WESNGQPENN YKTTPPVLDS
DGSFFLYSKL TVDKSRWQQG NVFSCSVMHE ALHNHYTQKS LSLSPGK.

Furthermore, the invention also provides a binding molecule having the property of binding to ALWGPDAAA (SEQ ID NO: 1) in complex with HLA-A*02, wherein the binding molecule comprises a first polypeptide chain, a second polypeptide chain and a third polypeptide chain comprising the amino acid sequences provided in SEQ ID NO: 47, SEQ ID NO: 54 and SEQ ID NO: 51 respectively.

The binding molecule may comprise
a) a first polypeptide chain having the following amino acid sequence:

(SEQ ID NO: 83)
AVQLVESGGG LVQPGGSLRL SCAASGFTFS SYAMTWVRQA PGKGPEWVSA IASDGASTSY
ADSVKGRFTI SRDNSKNTLY LQMNSLRPED TAVYYCARGG YLTYDRYGQG TLVTVSSGGG
GSNAGVTQTP KFRILKIGQS MTLQCAQDLQ HSYMYWYRQD PGMGLKPIYY SVGVGFTDKG
EVPQGYQVSR STTEDFPLRL ESAAPSQTSV YFCASAYMTG ELFFGEGSRL TVLEDLKNVF
PPEVAVFEPS EAEISHTQKA TLVCLATGFY PDHVELSWWV NGKEVHSGVC TDPQPLKEQP
ALQDSRYALS SRLRVSATFW QDPRNHFRCQ VQFYGLSEND EWTQDRAKPV TQIVSAEAWG
RAD;

b) a second polypeptide chain having the following amino acid sequence:

(SEQ ID NO: 100)
AKEVEQNSGP LSVPEGAIAS LQCTYSDKHS QGFFWYRQYS GKSPELIMSI YSQGDKEDGR
FTAQLNKASQ YVSLLIRDSQ PSDSATYLCA VRGNEKLTFG TGTRLTIIPN IQNPDPAVYQ
LRDSKSSDKS VCLFTDFDSQ TQVSQSKDSD VYITDKCVLD MRSMDFKSNS AVAWSQKSDF
ACANAFQNSI IPEDTDKTHT CPPCPAPELL GGPSVFLFPP KPKDTLYITR EPEVTCVVVD
VSHEDPEVKF NWYVDGVEVH NAKTKPREEQ YGSTYRVVSV LTVLHQDWLN GKEYKCKVSN
KALPAPIEKT ISKAKGQPRE PQVYTLPPSR DELTKNQVSL SCAVKGFYPS DIAVEWESNG
QPENNYKTTP PVLDSDGSFF LVSKLTVDKS RWQQGNVFSC SVMHEALHNH YTQKSLSLSP
GK;

and
c) a third polypeptide chain having the following amino acid sequence:

(SEQ ID NO: 98)
DKTHTCPPCP APELLGGPSV FLFPPKPKDT LYITREPEVT CVVVDVSHED PEVKFNWYVD
GVEVHNAKTK PREEQYGSTY RVVSVLTVLH QDWLNGKEYK CKVSNKALPA PIEKTISKAK
GQPREPQVYT LPPSRDELTK NQVSLWCLVK GFYPSDIAVE WESNGQPENN YKTTPPVLDS
DGSFFLYSKL TVDKSRWQQG NVFSCSVMHE ALHNHYTQKS LSLSPGK.

Furthermore, the invention also provides a binding molecule having the property of binding to ALWGPDAAA (SEQ ID NO: 1) in complex with HLA-A*02, wherein the binding molecule comprises a first polypeptide chain, a second polypeptide chain and a third polypeptide chain comprising the amino acid sequences provided in SEQ ID NO: 83, SEQ ID NO: 100 and SEQ ID NO: 98 respectively.

The binding molecules of the invention are preferably comprised of protein. The binding molecule may be glycosylated, amidated, carboxylated, phosphorylated, esterified, N-acylated, cyclized via, e.g., a disulfide bridge, or converted into an acid addition salt and/or optionally dimerized or polymerized, or conjugated. All such forms are encompassed by the present invention.

The binding molecule may comprise one or more substitutions in the TCR alpha chain variable or constant domain and/or the TCR beta chain variable or constant domain which remove one or more glycosylation sites, such as N-linked glycosylation sites. The substitutions in this context are relative to a native (e.g., wild-type) TCR (e.g., comprising an alpha chain of SEQ ID NO: 2 and a beta chain of SEQ ID NO: 12). For example, the binding molecule may comprise completely deglycosylated (i.e., aglycosylated) TCR chains, i.e., no N-linked glycosylation sites present in the alpha chain and beta chain sequences. In this regard, the inventors surprisingly found that fully deglycosylated TCR sequences resulted in increased potency relative to a fully glycosylated equivalent sequence.

The binding molecules may be synthetic, recombinant, isolated, engineered and/or purified. By “purified” it is meant, when referring to a polypeptide, or nucleotide sequence, that the indicated molecule is present in the substantial absence of other biological macromolecules of the same type. The term “purified” as used herein means that at least 75%, 85%, 95%, or 98% by weight, of biological macromolecules of the same type are the indicated molecule. A purified nucleic acid molecule that encodes a particular polypeptide refers to a nucleic acid molecule that is substantially free of other nucleic acid molecules that do not encode the subject polypeptide; however, the molecule may include some additional bases or moieties, which do not deleteriously affect the basic characteristics of the composition.

The binding molecule may be isolated. The term “isolated” means altered or removed from its natural state. For example, a nucleic acid or a polypeptide naturally present in a living animal is not “isolated,” but the same nucleic acid or peptide partially or completely separated from the coexisting materials of its natural state is “isolated”. An isolated nucleic acid or protein can exist in substantially purified form, or can exist in a non-native environment such as, for example, a host cell. An isolated binding molecule is substantially free of other binding molecules having different antigenic specificities. Moreover, an isolated binding molecule may be substantially free of other cellular material and/or chemicals.

The binding molecule may be recombinant. A “recombinant” molecule is one that has been prepared, expressed, created, or isolated by recombinant means. In this regard, recombinant molecules do not exist in nature.

### Single Domain Antibodies

Also provided by the present invention is a single domain antibody that binds to PD-1 and comprises CDRs, CDR1, CDR2 and CDR3, having the following amino acid sequences:
CDR1—GFTFSSYA (SEQ ID NO: 43), optionally with one, two or three mutations therein;
CDR2—IASDGAST (SEQ ID NO: 44), optionally with one, two or three mutations therein; and
CDR3—CARGG YLTYDRY (SEQ ID NO: 45), optionally with one, two or three mutations therein.

The single domain antibody of the invention may be a PD-1 agonist and/or may comprise any one or more of the features described above in relation to the immune suppressor of the binding molecule of the invention.

For example, the single domain antibody of the invention may be isolated and/or recombinant and/or soluble and/or humanised. The single domain antibody of the invention may be a VHH. Furthermore, the single domain antibody preferably does not compete with PD-L1 for binding to PD-1.

The single domain antibody of the invention may comprise the amino acid sequence provided in SEQ ID NO: 42 or a humanised version thereof, or an amino acid sequence having at least 80%, at least 90%, at least 95% or at least 98% identity to SEQ ID NO: 42. Alternatively, the single domain antibody of the invention may comprise the amino acid sequence of SEQ ID NO: 71, or an amino acid sequence having at least 80%, at least 90%, at least 95% or at least 98% identity to SEQ ID NO: 71.

Preferably, the single domain antibody of the invention comprises the amino acid sequence of SEQ ID NO: 71. In this regard, the present invention also provides a protein comprising the amino acid sequence of SEQ ID NO: 71.

The single domain antibody may have an affinity (i.e., K_{D}) for PD-1 of from about 1 nM to about 500 nM or preferably from about 50 nM to about 70 nM. The single domain antibody may have a binding half life of from about 1 seconds to about 40 seconds, or preferably from about 15 seconds to about 20 seconds. The affinity of the single domain antibody may be measured using methods described above for the binding molecule of the invention. The affinity of the single domain antibody may be measured using the methods provided in Example 1 or Example 4, for example.

The single domain antibody may have a high degree of specificity for PD-1 and give rise to a potent inhibitory response when tested in reporter assays as described in Example 4.

The single domain antibody may bind to an epitope in PD-1 comprising one or more or all of the following amino acids: E38, F59, P60, E61, T75, Q76, L77, P78, N79 and G80, numbered according to SEQ ID NO: 101. For example, the single domain antibody may bind to an epitope in PD-1 comprising at least 5, at least 6, at least 7, at least 8, or at least 9 of the following amino acids: E38, F59, P60, E61, T75, Q76, L77, P78, N79 and G80, numbered according to SEQ ID NO: 101. The single domain antibody may bind to an epitope in PD-1 comprising the following amino acids: E38, F59, P60, E61, T75, Q76, L77, P78, N79 and G80, numbered according to SEQ ID NO: 101.

The present invention also provides a binding molecule comprising
(a) the single domain antibody of the invention;
(b) a pMHC-binding domain, optionally comprising (i) an alpha chain, comprising at least a TCR alpha chain variable domain, and (ii) a beta chain, comprising at least a TCR beta chain variable domain; and
(c) optionally, a half-life extending domain.

The pMHC-binding domain and/or half-life extending domain may comprise any one or more features described herein.

### Amino Acid Sequences

Within the scope of the invention are phenotypically silent variants of any molecule disclosed herein. As used herein the term “phenotypically silent variants” is understood to refer to a variant which incorporates one or more further amino acid changes, including substitutions, insertions and deletions, in addition to those set out above, and which variant has a similar phenotype to the corresponding molecule without said change(s). For the purposes of this invention, phenotype comprises binding affinity (K_{D} and/or binding half-life) and/or specificity. The phenotype for a binding molecule may include potency of immune activation and purification yield, in addition to binding affinity and specificity. A phenotypically silent variant may have a K_{D} and/or binding half-life for the ALWGPDPAAA (SEQ ID NO: 1) HLA-A*02 complex within 50%, or more preferably within 30%, 25% or 20%, of the measured K_{D} and/or binding half-life of the corresponding binding molecule without said change(s), when measured under identical conditions (for example at 25° C. and/or on the same SPR chip). Suitable conditions are further provided in the Examples.

Furthermore, a phenotypically silent variant may retain the same, or substantially the same, therapeutic window between binding to the ALWGPDPAAA (SEQ ID NO: 1) HLA-A*02 complex and binding to one or more alternative peptide-HLA complexes. A phenotypically silent variant may retain the same, or substantially the same, therapeutic window between potency of immune cell inhibition in response to cells presenting to the ALWGPDPAAA (SEQ ID NO: 1) HLA-A*02 complex and cells presenting one or more alternative off-target peptide-HLA complexes. The therapeutic window may be calculated based on lowest effective concentrations (“LOEL”) observed for normal cells and the indication relevant cell line. The therapeutic window may be at least 10 fold different; at least 100 fold difference, at least 1000 fold difference, or more. A phenotypic variant may share the same, or substantially the same recognition motif as determined by sequential mutagenesis techniques discussed further below.

As is known to those skilled in the art, it may be possible to produce binding molecules that incorporate changes in the variable domains thereof compared to those detailed above without significantly altering the affinity of the interaction with the ALWGPDPAAA (SEQ ID NO: 1) HLA-A*02 complex, and or other functional characteristics. In particular, such silent mutations may be incorporated within parts of the sequence that are known not to be directly involved in antigen binding (e.g. the framework regions and or parts of the CDRs that do not contact the antigen). Such variants are included in the scope of this invention.

Phenotypically silent variants may contain one or more conservative substitutions and/or one or more tolerated substitutions. By tolerated substitutions it is meant those substitutions which do not fall under the definition of conservative as provided below but are nonetheless phenotypically silent. The skilled person is aware that various amino acids have similar properties and thus are ‘conservative’. One or more such amino acids of a protein, polypeptide or peptide can often be substituted by one or more other such amino acids without eliminating a desired activity of that protein, polypeptide or peptide.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). It should be appreciated that amino acid substitutions within the scope of the present invention can be made using naturally occurring or non-naturally occurring amino acids. For example, it is contemplated herein that the methyl group on an alanine may be replaced with an ethyl group, and/or that minor changes may be made to the peptide backbone. Whether or not natural or synthetic amino acids are used, it is preferred that only L-amino acids are present.

Substitutions of this nature are often referred to as “conservative” or “semi-conservative” amino acid substitutions. The present invention therefore extends to use of a molecule comprising any of the amino acid sequences described above but with one or more conservative substitutions and or one or more tolerated substitutions in the sequence, such that the amino acid sequence of the molecule, or any domain or region thereof, has at least 90% identity, such as 90%, 91%, 92%, 93%, 94%, 95%, 96%, 97%, 98%, 99% or 100% identity, to the sequences disclosed herein.

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)).

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The BLASTn and BLASTp programs of Altschul, et al. (1990) J. Mol. Biol. 215:403-410 have incorporated such an algorithm. Determination of percent identity between two nucleotide sequences can be performed with the BLASTn program. Determination of percent identity between two protein sequences can be performed with the BLASTp program. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., BLASTp and BLASTp) can be used. See http://www.ncbi.nlm.nih.gov. Default general parameters may include for example, Word Size=3, Expect Threshold=10. Parameters may be selected to automatically adjust for short input sequences. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search. For the purposes of evaluating percent identity in the present disclosure, BLASTp with the default parameters is used as the comparison methodology. In addition, when the recited percent identity provides a non-whole number value for amino acids (i.e., a sequence of 25 amino acids having 90% sequence identity provides a value of “22.5”, the obtained value is rounded down to the next whole number, thus “22”). Accordingly, in the example provided, a sequence having 22 matches out of 25 amino acids is within 90% sequence identity.

As used herein, where a sequence is referred to as having sequence identity to another sequence, that sequence retains the function, e.g. the general binding characteristics in the case of a peptide, of the other sequence.

As will be obvious to those skilled in the art, it may be possible to truncate, or extend, the sequences provided at the C-terminus and/or N-terminus thereof, by 1, 2, 3, 4, 5 or more residues, without substantially affecting the functional characteristics of the molecule, for example a TCR portion. The sequences provided at the C-terminus and/or N-terminus thereof may be truncated or extended by 1, 2, 3, 4 or 5 residues. All such variants are encompassed by the present invention.

Mutations, including conservative and tolerated substitutions, insertions and deletions, may be introduced into the sequences provided using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning—A Laboratory Manual (3^{rd} Ed.) CSHL Press. Further information on ligation independent cloning (LIC) procedures can be found in Rashtchian, (1995) Curr Opin Biotechnol 6(1):30-6. The protein sequences provided herein may be obtained from recombinant expression, solid state synthesis, or any other appropriate method known in the art.

### Assessing Binding Characteristics and Activity of Binding Molecules

Methods to determine binding affinity (inversely proportional to the equilibrium constant K_{D}) and binding half-life (expressed as T_{1/2}) are known to those skilled in the art. Binding affinity and binding half-life may be determined using Surface Plasmon Resonance (SPR) or Bio-Layer Interferometry (BLI), for example using a BIAcore instrument or Octet instrument, respectively. For example, binding affinity of a binding molecule for a peptide-MHC complex may be determined using SPR at 25° C., and/or at 37° C., wherein the peptide-MHC complex is immobilised on a solid support (e.g., a sensor chip) and is contacted with a solution comprising the binding molecule. Suitable experimental conditions and methods for determining binding parameters are described in the Examples (e.g., Example 1 and Example 2).

It will be appreciated by those skilled in the art that a higher affinity refers to a lower numerical value for K_{D} and indicates stronger binding. In other words, a doubling of affinity refers to halving the numerical value of the K_{D}. T_{1/2} is calculated as In2 divided by the off-rate (k_{off}). Therefore, doubling of T_{1/2} results in a halving in k_{off}. K_{D} and k_{off} values for TCRs are usually measured for soluble forms of the TCR, i.e. those forms which are truncated to remove cytoplasmic and transmembrane domain residues. To account for variation between independent measurements, and particularly for interactions with dissociation times in excess of 20 hours, the binding affinity and or binding half-life of a given protein may be measured several times, for example 3 or more times, using the same assay protocol, and an average of the results taken. To compare binding data between two samples (i.e. two different proteins and or two preparations of the same protein) it is preferable that measurements are made using the same assay conditions (e.g. temperature). Measurement methods described in relation to TCRs may also be applied to the binding molecules described herein.

Certain binding molecules of the invention, i.e., those comprising an immune checkpoint agonist, are able to generate potent inhibition of a T cell response in vitro against antigen positive cells. The T cell response that is measured may be the release of T cell activation markers such as Interferon γ or Granzyme B, or target cell killing, or other measure of T cell activation, such as T cell proliferation. Other methods of assessing inhibition of T cell activation include the Jurkat NFAT cell reporter assay described in Example 4.

Molecules encompassed by the present invention may have an improved half-life. Methods for determining whether a protein has an improved half-life will be apparent to the skilled person. For example, the ability of a protein to bind to a neonatal Fc receptor (FcRn) is assessed. In this regard, increased binding affinity for FcRn increases the serum half-life of the protein (see for example, Kim et al. Eur J Immunol., 24:2429, 1994).

The half-life of a protein disclosed herein can also be measured by pharmacokinetic studies, e.g., according to the method described by Kim et al. Eur J of Immunol 24:542, 1994. According to this method radiolabeled protein is injected intravenously into mice and its plasma concentration is periodically measured as a function of time, for example at 3 minutes to 72 hours after the injection. Alternatively, an unlabelled binding molecule of the invention can be injected and its plasma concentration periodically measured using an ELISA. The clearance curve thus obtained should be biphasic, that is, an alpha phase and beta phase. For the determination of the in vivo half-life of the protein, the clearance rate in beta-phase is calculated and compared with that of the wild type or unmodified protein.

### Nucleic Acids, Vectors and Host Cells

The present invention provides a nucleic acid encoding a binding molecule or single domain antibody of the invention. The alpha and beta chains of the binding molecule may be encoded within a single open reading frame, or within two distinct open reading frames. Similarly, for binding molecules comprising three polypeptide chains, the polypeptide chains may be encoded within a single open reading frame or three distinct open reading frames. Alternatively, the alpha and beta chains, or the two or three polypeptide chains, of the binding molecule may be encoded on separate nucleic acids. The term “nucleic acid” includes but is not limited to ribonucleic acid (RNA) and deoxyribonucleic acid (DNA) molecules, which may be single or double stranded. The nucleic acid may be present in whole cells, in a cell lysate, or may be in an isolated, partially purified or substantially pure form. A nucleic acid is rendered “substantially pure” when purified away from other cellular components or other contaminants, e.g., other cellular nucleic acids or proteins, by standard techniques. The nucleic acid may be recombinant and/or non-naturally occurring and/or engineered. The nucleic acid sequence may be codon optimised, in accordance with the expression system utilised. As is known to those skilled in the art, expression systems may include bacterial cells such as E. coli, or yeast cells, or mammalian cells, or insect cells, or they may be cell free expression systems.

The present invention also provides constructs in the form of plasmids, vectors, transcription or expression cassettes which comprise at least one nucleic acid as described above. In particular, the invention provides an expression vector comprising the nucleic acid of the invention. The terms “vector”, “cloning vector” and “expression vector” refer to a vehicle by which a DNA or RNA sequence (e.g. a foreign gene) can be introduced into a host cell, so as to transform the host and optionally promote expression (e.g. transcription and translation) of the introduced sequence. The vector may be capable of expressing in T cells both Foxp3 and a binding molecule of the invention.

The alpha and beta chains of the binding molecule of the invention may be expressed together with Foxp3 (optionally a GFP/Foxp3 fusion protein). For example, the binding molecule of the invention and Foxp3 may be expressed from a multicistronic retroviral vector using, for example, a viral ribosome skip (2A) and internal ribosome entry sites (IRES). Vectors of this type may efficiently convert conventional CD4⁺ T cells into antigen specific regulatory phenotype T cells (Treg). Without wishing to be bound by theory, it is expected that co-delivery of an islet-antigen specific binding molecule of the invention and Foxp3 ensures islet specificity is not dissociated from regulatory activity and therefore enables the transfected T cells to exercise optimal control over the pro-inflammatory environment which otherwise supports the destruction of the islet cells.

The present invention also provides a recombinant host cell which comprises one or more the constructs as above. As mentioned, a nucleic acid encoding a binding molecule, or single domain antibody, of the invention forms an aspect of the present invention, as does a method of production of the binding molecule or single domain antibody comprising expression from a nucleic acid encoding the binding molecule or single domain antibody of the invention. Expression may conveniently be achieved by culturing recombinant host cells containing the nucleic acid under appropriate conditions. Following production by expression, a binding molecule or single domain antibody may be isolated and/or purified using any suitable technique, then used as appropriate.

Systems for cloning and expression of a protein, such as a binding molecule or single domain antibody of the invention, in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli. The expression of TCRs, antibodies and antibody fragments in prokaryotic cells such as E. coli is well established in the art. For a review, see for example Pluckthun, Bio/Technology 9:545-551 (1991). Expression in eukaryotic cells in culture is also available to those skilled in the art as an option for production of a binding molecule, see for recent review, for example Reff, Curr. Opinion Biotech. 4:573-576 (1993); Trill et al., Curr. Opinion Biotech. 6:553-560 (1995).

Suitable vectors can be chosen or constructed, containing appropriate regulatory sequences, including promoter sequences, terminator sequences, polyadenylation sequences, enhancer sequences, marker genes and other sequences as appropriate. Vectors may be any suitable vectors known in the art, including plasmids or viral vectors (e.g. ‘phage, or phagemid), as appropriate. For further details see, for example, Sambrook et al., Molecular Cloning: A Laboratory Manual: 2nd Edition, Cold Spring Harbor Laboratory Press (1989). Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al. eds., Short Protocols in Molecular Biology, 2nd Edition, John Wiley & Sons (1992).

The present invention also provides a host cell containing a nucleic acid as disclosed herein. The invention also provides a cell harbouring
(a) an expression vector of the invention;
(b) a first expression vector comprising a nucleic acid encoding a first polypeptide comprising the alpha chain of a binding molecule of the invention and a second expression vector comprising a nucleic acid encoding a second polypeptide comprising the beta chain of a binding molecule of the invention; or
(c) a first expression vector comprising a nucleic acid encoding the first polypeptide of a three-chain binding molecule described herein, a second expression vector comprising a nucleic acid encoding the second polypeptide of a three-chain binding molecule described herein, and a third expression vector comprising a nucleic acid encoding the third polypeptide of a three-chain binding molecule described herein.

Also provided is a non-naturally occurring and/or purified and/or engineered cell, preferably a T-cell, presenting the binding molecule of the invention. There are a number of methods suitable for the transfection of T cells with nucleic acid (such as DNA or RNA) encoding the TCRs of the invention (see for example Robbins et al., 2008 J Immunol. 180:6116-6131 and Plesa et al. 2012 Blood. 119(15):3420-3430). The cell may be a CD4+ and/or Foxp3+ T cell. The cell may be a Treg cell, for example. Such cells presenting a binding molecule of the invention may be used in adoptive therapy for treating diabetes.

Further, the invention provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene.

Suitable host cells for cloning or expression of proteins from nucleic acids and/or vectors of the present invention are known in the art. Suitable host cells for the expression of (glycosylated) proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse Sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for protein production, see, e.g., Yazaki, P. and Wu, A. M., Methods in Molecular Biology, Vol. 248, Lo, B. K. C. (ed.), Humana Press, Totowa, NJ (2004), pp. 255-268. The host cell may be eukaryotic, e.g., a Chinese Hamster Ovary (CHO) cell or lymphoid cell (e.g., Y0, NS0, Sp20 cell). Alternatively, the host cell may be prokaryotic, e.g., an E. coli cell.

The nucleic acid of the invention may be integrated into the genome (e.g. chromosome) of the host cell. Integration may be promoted by inclusion of sequences which promote recombination with the genome, in accordance with standard techniques.

### Methods of Making Binding Molecules

Further provided herein are methods for producing a binding molecule or a single domain antibody of the invention. In one aspect, the methods comprise a) maintaining a cell of the invention under conditions suitable for expression of the binding molecule or single domain antibody, and b) isolating the binding molecule or a single domain antibody.

Methods of producing recombinant proteins, such as a binding molecules or a single domain antibody of the invention, are well known in the art. Nucleic acids encoding the protein can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary mammalian cells used for expressing a protein are CHO cells, myeloma cells or HEK cells. Preferred cells for producing the binding molecules of the invention are E. coli cells. Molecular cloning techniques to achieve these ends are known in the art and described, for example in Ausubel et al. (editors), Current Protocols in Molecular Biology, Greene Pub. Associates and Wiley-Interscience (1988, including all updates until present) or Sambrook et al. Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

The nucleic acid may be operably linked to a promoter in an expression construct or expression vector for further cloning (amplification of the DNA) or for expression in a cell-free system or in cells. As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid. As used herein, the term “operably linked to” means positioning a promoter relative to a nucleic acid such that expression of the nucleic acid is controlled by the promoter.

Many vectors for expression in cells are commercially available. The vector components generally include, but are not limited to, one or more of the following: a signal sequence, a sequence encoding a protein (e.g., derived from the information provided herein), an enhancer element, a promoter, and a transcription termination sequence. The skilled person will be aware of suitable sequences for expression of a protein. Exemplary signal sequences include prokaryotic secretion signals (e.g., pe1B, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-a promoter (EF1), small nuclear RNA promoters (Ula and Ulb), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or an active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GALA promoter, the CUP1 promoter, the PH05 promoter, the nmt promoter, the RPR1 promoter, or the TEF1 promoter.

The host cells used to produce the protein may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

Methods for isolating a protein are known in the art. Where a protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing.

These methods are known in the art and described, for example in WO99/57134 or Ed Harlow and David Lane (editors) Antibodies: A Laboratory Manual, Cold Spring Harbor Laboratory, (1988). The skilled person will also be aware that a protein can be modified to include a tag to facilitate purification or detection, e.g., a poly-histidine tag, a hexa-histidine tag, an influenza virus hemagglutinin (HA) tag, a Simian Virus 5 (V5) tag, a LLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Molecules of the invention may be amenable to high yield purification. Yield may be determined based on the amount of material retained during the purification process (i.e. the amount of correctly folded material obtained at the end of the purification process relative to the amount of solubilised material obtained prior to refolding), and or yield may be based on the amount of correctly folded material obtained at the end of the purification process, relative to the original culture volume. High yield means greater than 1%, or greater than 5%, or higher yield. High yield means greater than 1 mg/ml, or greater than 3 mg/ml, or greater than 5 mg/ml, or higher yield.

### Pharmaceutical Compositions and Medical Methods

The binding molecules, single domain antibodies, nucleic acids, expression vectors and/or cells of the invention may be used in a method of treating or diagnosing an autoimmune disease, such as type 1 diabetes. For administration to patients, the binding molecules, single domain antibodies, nucleic acids, expression vectors and/or cells of the invention may be provided as part of a pharmaceutical composition together with one or more pharmaceutically acceptable carriers or excipients (for example a buffering agent, also known as a “buffer”). This pharmaceutical composition may be in any suitable form, (e.g. depending upon the desired method of administering it to a patient). It may be provided in unit dosage form and will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, such as parenteral (including subcutaneous, intramuscular, intrathecal or intravenous), enteral (including oral or rectal), inhalation or intranasal routes. For example, the pharmaceutical composition may be adapted (e.g., formulated) for subcutaneous administration. Such compositions may be prepared by any method known in the art, for example by mixing the active ingredient with the carrier(s) or excipient(s) under sterile conditions. Methods for preparing a protein into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions will commonly comprise a solution of the binding molecule of the invention (or the nucleic acid, cell, or vector of the invention) dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of binding molecules and single domain antibodies of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Binding molecules, single domain antibodies, pharmaceutical compositions, vectors, nucleic acids and cells of the invention may be provided in substantially pure form, for example, at least 80%, at least 85%, at least 90%, at least 91%, at least 92%, at least 93%, at least 94%, at least 95%, at least 96%, at least 97%, at least 98%, at least 99% or 100% pure.

Binding molecules of the invention may have an ideal safety profile for use as therapeutic reagents. “Safety profile”, as used herein, refers to the capacity to distinguish a antigen positive cell, in particular a ALWGPDPAAA (SEQ ID NO: 1)-HLA-A*02 complex presenting cell, from antigen negative cells. This capacity is often expressed by indication of a “safety window” or “therapeutic window”. In this case the binding molecules may be in soluble form and may preferably be fused to an immune suppressor. Suitable immune suppressors are described herein and include but are not limited to, immune checkpoint agonists, interleukins, cytokines, antibodies and antibody like scaffolds, including fragments, derivatives and variants thereof that bind to antigens on immune cells such as T cells, B cells or NK cells (e.g. anti-PD-1 agonist antibodies). An ideal safety profile means that in addition to demonstrating good specificity, the binding molecules of the invention may have passed further preclinical safety tests. Examples of such tests include whole blood assays to confirm minimal cytokine release in the presence of whole blood and thus low risk of causing a potential cytokine release syndrome in vivo, and alloreactivity tests to confirm low potential for recognition of alternative HLA types.

Suitable dosages of the binding molecules or a single domain antibodies of the present invention can vary between wide limits, depending upon the disease or disorder to be treated, the age and condition of the subject to be treated, etc. Preferably, the subject is a human. A physician may ultimately determine appropriate dosages to be used. Administration of the binding molecule or single domain antibody may be in a “therapeutically effective amount,” this being an amount sufficient to show benefit to the patient.

In another aspect, the invention provides a binding molecule, single domain antibody, nucleic acid, vector, pharmaceutical composition or cell of the invention for use in medicine. In particular, the binding molecule, a single domain antibody, nucleic acid, vector, pharmaceutical composition and cell of the invention may be used for treating autoimmune diseases such as diabetes. Thus, also provided by the invention is a binding molecule, a single domain antibody, nucleic acid, vector, pharmaceutical composition or cell of the invention for use in a method of treating diabetes. The diabetes to be treated is preferably type 1 diabetes mellitus (T1DM). For example, the method of treating diabetes may comprise administering a soluble binding molecule, single domain antibody, or pharmaceutical composition of the invention or may be an adoptive therapy method comprising administering a cell of the invention (such as a Treg cell presenting the binding molecule of the invention). As will be known to those skilled in the art, there are a number of suitable methods by which adoptive therapy can be carried out (see for example Rosenberg et al., 2008 Nat Rev Cancer 8 (4): 299-308).

Also provided by the invention are:
a binding molecule, single domain antibody, nucleic acid, vector, pharmaceutical composition or cell of the invention for use in medicine, preferably for use in a human subject and/or preferably for use in a method of treating diabetes;
a binding molecule, single domain antibody, nucleic acid, vector, pharmaceutical composition or cell of the invention for use in a method of diagnosing diabetes in a subject;
use of a binding molecule, a single domain antibody, nucleic acid, vector, pharmaceutical composition or cell of the invention in the manufacture of a medicament for treating diabetes;
a method of treating diabetes in a subject, comprising administering to the subject a binding molecule, a single domain antibody, nucleic acid, vector, pharmaceutical composition or cell of the invention;
an injectable formulation for administering to a human subject comprising a binding molecule, a single domain antibody, nucleic acid, vector pharmaceutical composition or cell of the invention.

### Kits and Articles of Manufacture

In another aspect, a kit or an article of manufacture containing materials useful for the treatment, diagnosis and/or prevention of the diseases described above is provided.

The kit may comprise (a) a container comprising the binding molecule, a single domain antibody, nucleic acid, vector or cell of the invention, optionally in a pharmaceutically acceptable carrier or diluent; and (b) a package insert with instructions for treating or diagnosing a disease (e.g., diabetes) in a subject. The kit may further comprise (c) at least one further therapeutically active compound or drug.

The package insert may be on or associated with the container. Suitable containers include, for example, bottles, vials, syringes, etc. The containers may be formed from a variety of materials such as glass or plastic. The container holds or contains a composition that comprises the binding molecule, a single domain antibody, nucleic acid, vector or cell of the invention and may have a sterile access port (for example, the container may be an intravenous solution bag or a vial having a stopper pierceable by a hypodermic injection needle). At least one active agent in the composition is the binding molecule, a single domain antibody, nucleic acid, vector or cell of the invention. The label or package insert may indicate that the composition is used for treating a subject eligible for treatment, e.g., one having or predisposed to developing a disease described herein, with specific guidance regarding dosing amounts and intervals of the composition and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The invention also includes particles displaying binding molecules or single domain antibodies of the invention and the inclusion of said particles within a library of particles. Such particles include but are not limited to phage, yeast cells, ribosomes, or mammalian cells. Method of producing such particles and libraries are known in the art (for example see WO2004/044004; WO01/48145, Chervin et al. (2008) J. Immuno. Methods 339.2:175-184).

Various modifications of the invention in addition to those shown and described herein will become apparent to those skilled in the art from the foregoing description and fall within the scope of the appended claims. Preferred features of each aspect of the invention are as for each of the other aspects mutatis mutandis. The documents referred to herein are incorporated by reference to the fullest extent permitted by law.

### DESCRIPTION OF THE DRAWINGS

FIG. 1: Graphs showing recognition of PPI peptide and mimetic peptides by soluble native TCRs

FIG. 2: Graphs showing recognition of serine substituted PPI peptide by native soluble TCRs

FIG. 3: Schematic of an exemplary TCR-PD-1 agonist binding molecule incorporating an Fc domain

FIG. 4: a) Graphs showing inhibition of T cell signalling in Jurkat NFAT reporter assay by TCR PD-1 agonist binding molecules b) A graph comparing inhibition of T cell signalling in Jurkat NFAT reporter assay by TCR PD-1 agonist with and without Fc.

FIG. 5: A graph showing in vivo concentration of TCR PD-1 agonist binding molecule in SCID mice over 3 weeks following intravenous (IV) or subcutaneous (SC) administration.

FIG. 6: A graph showing inhibition of IL2 release by primary CD4⁺ T cells in the presence of TCR PD-1 agonist

FIG. 7: Graphs showing inhibition of B cell killing (a) and IFNγ cytokine release (b) by two autoreactive T cell clones in the presence of TCR PD-1 agonist binding molecule

FIG. 8: Graphs showing inhibition of stimulation in PD1+ve and PD-1−ve NK cells indicated by % cells positive for CD107a and IFNγ, in the presence of TCR PD-1 agonist binding molecules (*p≤0.05, **p≤0.01; ns=not significant).

FIG. 9: Images showing results of experiments analysing the specificity of the selected PD-1 agonist VHH described in Example 4 using Retrogenix Cell Microarray Technology. The VHH bound to PD-1 but not the other control proteins in the microarray.

FIG. 10: TCR-PD-1 agonist inhibits NK-92-PD-1 cells stimulated in-vitro with HLA-A*02+ve K562 target cells pulsed with PPI peptide. (a) Schematic of experimental model. (b) Representative flow cytometry profile and graph showing specific and concentration dependent binding of TCR-PD-1 agonist molecules to target cells. (c) Graph showing % inhibition of CD107a expression and IFNγ intracellular production by NK92-PD-1 cells stimulated with target cells and incubated with different concentration of control (grey dot) or TCR-PD-1 agonist (white dot). Each dot represents the mean of 3 independent experiments, n=6, two-way ANOVA, **p≤0.01, ****p≤0.0001). (d) Representative flow cytometry profiles and graphs showing CD107a expression and IFNγ intracellular production by NK92-PD-1 cells stimulated with target cells in presence of 10 nM control (grey) or 10 nM TCR-PD-1 agonist (white). Each dot represents one sample, n=6, 3 independent experiments, Paired t-test, ***p≤0.001, ****p≤0.0001).

FIG. 11: TCR-PD-1 agonist interaction with CD4 T cell leads to a prolonged modulation of the T cell response. (a) Schematic of experimental model. (b) Graph showing IL-2 produced during activation from day 0 to day 3 by CD4 T cells in presence (white) or absence (grey) of TCR-PD-1 agonist. Each dot represents one donor, n=12, 5 independent experiments, Paired t test, *p≤0.05. (c) Graph showing IL-2 produced from day 8 to day 11 by CD4 T cells previously activated with (white) or without (grey) TCR-PD-1 agonist. Each dot represents one donor, n=8, 3 independent experiments, Paired t test, **p≤0.01. (d) Graph showing IL-2 produced from day 8 to day 11 by CD4 T cells re-activated in presence (white) or absence (grey) of TCR-PD-1 agonist. Each dot represents one donor, n=8, 3 independent experiments, Paired t test, **p≤0.01.

### DESCRIPTION OF THE SEQUENCES

### HLA-A*02 Restricted Peptides

Source protein gene is indicated in brackets below.
SEQ ID NO: 1 (Pre-Pro Insulin 15-24; Uniprot ref: P01308): ALWGPDPAAA
SEQ ID NO: 67 (Basic helix-loop-helix family, member e41, “Mim1”): ALLGPDPAAA
SEQ ID NO: 88 (nuclear DEAF-1 related transcriptional regulator protein 8, “Mim2”): ALPGPDEAAA
SEQ ID NO: 89 (forkhead box protein F2, “Mim3”): ALMSPPPAAA
SEQ ID NO: 90 (myeloid zinc finger protein, “Mim4”): ALWDPGPEAA
SEQ ID NO: 91 (histone-lysine N-methyltransferase 2D, “Mim5”): ALGSPPPAAA

### Alpha Chain of an Exemplary Scaffold TCR (SEQ ID NO: 2)

QKEVEQNSGP LSVPEGAIAS LNCTYSDRGS QSFFWYRQYS GKSPELIMSI YSNGDKEDGR
FTAQLNKASQ YVSLLIRDSQ PSDSATYLCA VRGNEKLTFG TGTRLTIIPN IQNPDPAVYQ
LRDSKSSDKS VCLFTDFDSQ TNVSQSKDSD VYITDKCVLD MRSMDFKSNS AVAWSNKSDF
ACANAFNNSI IPEDT

SEQ ID NO: 2 is an amino acid sequence of the alpha chain of an exemplary wild type (e.g., “scaffold”) TCR (comprising the alpha chain of SEQ ID NO: 2 and the beta chain of SEQ ID NO: 12) that binds to ALWGPDPAAA (SEQ ID NO: 1) in complex with HLA-A*02. This TCR is referred to as “S2” herein. The alpha chain comprises a variable domain (SEQ ID NO: 3) and a constant domain (SEQ ID NO: 4, italics). CDRs (CDR1, CDR2 and CDR3) are underlined and are designated SEQ ID NO: 5, 6 and 7 respectively, framework regions (FR1, FR2, FR3 and FR4) are in regular text and are designated SEQ ID NO: 8, 9, 10 and 11 respectively. The constant domain includes a T48C (numbered according to SEQ ID NO: 4) mutation (relative to a wild type constant domain), in bold text, to introduce a non-native covalent disulphide bond between the alpha and beta chains.

### Beta Chain of an Exemplary Scaffold TCR (SEQ ID NO: 12)

NAGVTQTPKF RILKIGQSMT LQCAQDMNHN YMYWYRQDPG MGLKPIYYSV GAGITDKGEV
PNGYNVSRST TEFPLRLES AAPSQTSVYF CASSYMTGEL FFGEGSRLTV LEDLKNVFPP
EVAVFEPSEA EISHTQKATL VCLATGFYPD HVELSWWVNG KEVHSGVCTD PQPLKEQPAL
NDSRYALSSR LRVSATFWQD PRNHFRCQVQ FQDRAKPVTQ IVSAEAWGRA D

SEQ ID NO: 12 is an amino acid sequence of the beta chain of an exemplary wild type (e.g., “scaffold”) TCR (comprising the alpha chain of SEQ ID NO: 2 and the beta chain of SEQ ID NO: 12) that binds to ALWGPDPAAA (SEQ ID NO: 1) in complex with HLA-A*02. This TCR is referred to as “S2” herein. The beta chain comprises a variable domain (SEQ ID NO: 13) and a constant domain (SEQ ID NO: 14, italics). CDRs (CDR1, CDR2 and CDR3) are underlined and are designated SEQ ID NO: 15, 16 and 17 respectively, framework regions (FR1, FR2, FR3 and FR4) are in regular text and are designated SEQ ID NO: 18, 19, 20 and 21 respectively. The constant domain includes a S57C (numbered according to SEQ ID NO: 14) mutation (relative to a wild type constant domain), in bold text, to introduce a non-native covalent disulphide bond between the alpha and beta chains. Also in bold text is a C75A mutations (numbered according to SEQ ID NO: 14) which removes a native cysteine to decrease incorrect disulphide formation

### Exemplary Mutated TCR Alpha Chain Variable Domains

The following sequences are exemplary alpha chain variable domains which contain mutations relative to the wild type sequence in SEQ ID NO: 3, which were introduced to enhance affinity, stability and/or manufacturability. The CDRs are underlined and the mutations are shown in bold.

Alpha chain variable domain “a2” (SEQ ID NO: 22) comprising CDRs (CDR1, CDR2 and CDR3—underlined) designated SEQ ID NO: 23, 6 and 7, respectively, and framework regions (FR1, FR2, FR3 and FR4—regular text) designated SEQ ID NO: 8, 9, 10 and 11, respectively:

QKEVEQNSGP LSVPEGAIAS LNCTYSDKHS QGFFWYRQYS
GKSPELIMSI YSNGDKEDGR FTAQLNKASQ YVSLLIRDSQ
PSDSATYLCA VRGNEKLTFG TGTRLTIIP

Alpha chain variable domain “a18” (SEQ ID NO: 24) comprising CDRs (CDR1, CDR2 and CDR3—underlined) designated SEQ ID NO: 23, 6 and 7, respectively, and framework regions (FR1, FR2, FR3 and FR4—regular text) designated SEQ ID NO: 25, 9, 10 and 11, respectively:

AKEVEQNSGP LSVPEGAIAS LQCTYSDKHS QGFFWYRQYS
GKSPELIMSI YSNGDKEDGR FTAQLNKASQ YVSLLIRDSQ
PSDSATYLCA VRGNEKLTFG TGTRLTIIP

Alpha chain variable domain “a19” (SEQ ID NO: 26) comprising CDRs (CDR1, CDR2 and CDR3—underlined) designated SEQ ID NO: 23, 27 and 7, respectively, and framework regions (FR1, FR2, FR3 and FR4—regular text) designated SEQ ID NO: 25, 9, 10 and 11, respectively

AKEVEQNSGP LSVPEGAIAS LQCTYSDKHS QGFFWYRQYS
GKSPELIMSI YSQGDKEDGR FTAQLNKASQ YVSLLIRDSQ
PSDSATYLCA VRGNEKLTFG TGTRLTIIP

### Exemplary Mutated TCR Beta Chain Variable Domains

The following sequences are exemplary beta chain variable domains which contain mutations relative to the wild type sequence in SEQ ID NO: 12, which were introduced to enhance affinity, stability and/or manufacturability. The CDRs are underlined and the mutations are shown in bold.

Beta chain variable domain “b3” (SEQ ID NO: 68) comprising CDRs (CDR1, CDR2 and CDR3—underlined) designated SEQ ID NO: 28, 29 and 30 respectively, framework regions (FR1, FR2, FR3 and FR4—regular text) designated SEQ ID NO: 18, 19, 20 and 21 respectively:

NAGVTQTPKF RILKIGQSMT LQCAQDMNHS YMYWYRQDPG
MGLKPIYYSV GVGFTDKGEV PNGYNVSRST TEDFPLRLES
AAPSQTSVYF CASAYMTGEL FFGEGSRLTV L

Beta chain variable domain “b16” (SEQ ID NO: 31) comprising CDRs (CDR1, CDR2 and CDR3—underlined) designated SEQ ID NO: 32, 29 and 30, respectively, and framework regions (FR1, FR2, FR3 and FR4—regular text) designated SEQ ID NO: 18, 19, 33 and 21, respectively:

NAGVTQTPKF RILKIGQSMT LQCAQDMQHS YMYWYRQDPG
MGLKPIYYSV GVGFTDKGEV PNGYQVSRST TEDFPLRLES
AAPSQTSVYF CASAYMTGEL FFGEGSRLTV L

Beta chain variable domain “b19” (SEQ ID NO: 34) comprising CDRs (CDR1, CDR2 and CDR3—underlined) designated SEQ ID NO: 35, 29 and 30, respectively, and framework regions (FR1, FR2, FR3 and FR4—regular text) designated SEQ ID NO: 18, 19, 33 and 21, respectively:

NAGVTQTPKF RILKIGQSMT LQCAQDLQHS YMYWYRQDPG
MGLKPIYYSV GVGFTDKGEV PNGYQVSRST TEDEPLRLES
AAPSQTSVYF CASAYMTGEL FFGEGSRLTV L

Beta chain variable domain “b20” (SEQ ID NO: 74) comprising CDRs (CDR1, CDR2 and CDR3—underlined) designated SEQ ID NO: 35, 29 and 30, respectively, and framework regions (FR1, FR2, FR3 and FR4—regular text) designated SEQ ID NO: 18, 19, 75 and 21, respectively:

NAGVTQTPKF RILKIGQSMT LQCAQDLQHS YMYWYRQDPG
MGLKPIYYSV GVGFTDKGEV PQGYQVSRST TEDFPLRLES
AAPSQTSVYF CASAYMTGEL FFGEGSRLTV L

Beta chain variable domain “b21” (SEQ ID NO: 76) comprising CDRs (CDR1, CDR2 and CDR3—underlined) designated SEQ ID NO: 35, 29 and 30, respectively, and framework regions (FR1, FR2, FR3 and FR4—regular text) designated SEQ ID NO: 18, 19, 77 and 21, respectively:

NAGVTQTPKF RILKIGQSMT LQCAQDLQHS YMYWYRQDPG
MGLKPIYYSV GVGFTDKGEV PEGYQVSRST TEDFPLRLES
AAPSQTSVYF CASAYMTGEL FFGEGSRLTV L

Beta chain variable domain “b22” (SEQ ID NO: 78) comprising CDRs (CDR1, CDR2 and CDR3—underlined) designated SEQ ID NO: 35, 29 and 30, respectively, and framework regions (FR1, FR2, FR3 and FR4—regular text) designated SEQ ID NO: 18, 19, 79 and 21, respectively:

NAGVTQTPKF RILKIGQSMT LQCAQDLQHS YMYWYRQDPG
MGLKPIYYSV GVGFTDKGEV PDGYQVSRST TEDFPLRLES
AAPSQTSVYF CASAYMTGEL FFGEGSRLTV L

### Exemplary TCRs

The following sequences are TCRs comprising exemplary combinations of the alpha and beta chain variable domains provided above. Constant domains are shown in italics. The CDRs are underlined and the mutations relative to the scaffold TCR sequence (i.e., SEQ ID NO: 2 or 12) are shown in bold.

a2b3 TCR

TCR “a2b3” alpha chain sequence (SEQ ID NO: 70), comprising the a2 variable domain (SEQ ID NO: 22—regular text) described above and the constant domain (SEQ ID NO: 4—italics) from the scaffold TCR described above:

QKEVEQNSGP LSVPEGAIAS LNCTYSDKHS QGFFWYRQYS
GKSPELIMSI YSNGDKEDGR FTAQLNKASQ YVSLLIRDSQ
PSDSATYLCA VRGNEKLTFG TGTRLTIIPN IQNPDPAVYQ
LRDSKSSDKS VCLFTDEDSQ TNVSQSKDSD VYITDKCVLD
MRSMDFKSNS AVAWSNKSDF ACANAFNNSI IPEDT

TCR “a2b3” beta chain sequence (SEQ ID NO: 69), comprising the b3 variable domain (SEQ ID NO: 68—regular text) and the constant domain (SEQ ID NO: 14—italics) from the scaffold TCR described above:

NAGVTQTPKF RILKIGQSMT LQCAQDMNHS YMYWYRQDPG
MGLKPIYYSV GVGFTDKGEV PNGYNVSRST TEDFPLRLES
AAPSQTSVYF CASAYMTGEL FFGEGSRLTV LEDLKNVFPP
EVAVFEPSEA EISHTQKATL VCLATGFYPD HVELSWWVNG
KEVHSGVCTD PQPLKEQPAL NDSRYALSSR LRVSATFWQD
PRNHERCQVQ FYGLSENDEW TQDRAKPVTQ IVSAEAWGRA D

a18b16 TCR

TCR “a18b16” alpha chain sequence (SEQ ID NO: 36), comprising the a18 variable domain (SEQ ID NO: 24—regular text) described above and a mutated constant domain (SEQ ID NO: 37—italics):

AKEVEQNSGP LSVPEGAIAS LQCTYSDKHS QGFFWYRQYS
GKSPELIMSI YSNGDKEDGR FTAQLNKASQ YVSLLIRDSQ
PSDSATYLCA VRGNEKLTFG TGTRLTIIPN IQNPDPAVYQ
LRDSKSSDKS VCLFTDFDSQ TQVSQSKDSD VYITDKCVLD
MRSMDFKSNS AVAWSQKSDF ACANAFQNSI IPEDT

TCR “a18b16” beta chain sequence (SEQ ID NO: 38), comprising the b16 variable domain (SEQ ID NO: 31—regular text) and a mutated constant domain (SEQ ID NO: 39—italics):

NAGVTQTPKF RILKIGQSMT LQCAQDMQHS YMYWYRQDPG
MGLKPIYYSV GVGFTDKGEV PNGYQVSRST TEDFPLRLES
AAPSQTSVYF CASAYMTGEL FFGEGSRLTV LEDLKNVFPP
EVAVFEPSEA EISHTQKATL VCLATGFYPD HVELSWWVNG
KEVHSGVCTD PQPLKEQPALQD SRYALSSRLR VSATFWQDPR
NHFRCQVQFY GLSENDEWTQ DRAKPVTQIV SAEAWGRAD

a19b19 TCR

TCR “a19b19” alpha chain sequence (SEQ ID NO: 40), comprising the a19 variable domain (SEQ ID NO: 26—regular text) described above and a mutated constant domain (SEQ ID NO: 37—italics):

AKEVEQNSGP LSVPEGAIAS LQCTYSDKHS QGFFWYRQYS
GKSPELIMSI YSQGDKEDGR FTAQLNKASQ YVSLLIRDSQ
PSDSATYLCA VRGNEKLTFG TGTRLTIIPN IQNPDPAVYQ
LRDSKSSDKS VCLFTDFDSQ TQVSQSKDSD VYITDKCVLD
MRSMDFKSNS AVAWSQKSDF ACANAFQNSI IPEDT

TCR “a19b19” beta chain sequence (SEQ ID NO: 41), comprising the b19 variable domain (SEQ ID NO: 34—regular text) and a mutated constant domain (SEQ ID NO: 39—italics):

NAGVTQTPKF RILKIGQSMT LQCAQDLQHS YMYWYRQDPG
MGLKPIYYSV GVGFTDKGEV PNGYQVSRST TEDFPLRLES
AAPSQTSVYF CASAYMTGEL FFGEGSRLTV LEDLKNVFPP
EVAVFEPSEA EISHTQKATL VCLATGFYPD HVELSWWVNG
KEVHSGVCTD PQPLKEQPALQD SRYALSSRLR VSATFWQDPR
NHFRCQVQFY GLSENDEWTQ DRAKPVTQIV SAEAWGRAD

a19b20 TCR

TCR “a19b20” alpha chain sequence (SEQ ID NO: 40), comprising the a19 variable domain (SEQ ID NO: 26—regular text) described above and a mutated constant domain (SEQ ID NO: 37—italics):

AKEVEQNSGP LSVPEGAIAS LQCTYSDKHS QGFFWYRQYS
GKSPELIMSI YSQGDKEDGR FTAQLNKASQ YVSLLIRDSQ
PSDSATYLCA VRGNEKLTFG TGTRLTIIPN IQNPDPAVYQ
LRDSKSSDKS VCLFTDFDSQ TQVSQSKDSD VYITDKCVLD
MRSMDFKSNS AVAWSQKSDF ACANAFQNSI IPEDT

TCR “a19b20” beta chain sequence (SEQ ID NO: 80), comprising the b20 variable domain (SEQ ID NO: 74—regular text) and a mutated constant domain (SEQ ID NO: 39—italics):

NAGVTQTPKF RILKIGQSMT LQCAQDLQHS YMYWYRQDPG
MGLKPIYYSV GVGFTDKGEV PQGYQVSRST TEDFPLRLES
AAPSQTSVYF CASAYMTGEL FFGEGSRLTV LEDLKNVFPP
EVAVFEPSEA EISHTQKATL VCLATGFYPD HVELSWWVNG
KEVHSGVCTD PQPLKEQPALQD SRYALSSRLR VSATFWQDPR
NHFRCQVQFY GLSENDEWTQ

a19b21 TCR

TCR “a19b21” alpha chain sequence (SEQ ID NO: 40), comprising the a19 variable domain (SEQ ID NO: 26—regular text) described above and a mutated constant domain (SEQ ID NO: 37—italics):

AKEVEQNSGP LSVPEGAIAS LQCTYSDKHS QGFFWYRQYS
GKSPELIMSI YSQGDKEDGR FTAQLNKASQ YVSLLIRDSQ
PSDSATYLCA VRGNEKLTFG TGTRLTIIPN IQNPDPAVYQ
LRDSKSSDKS VCLFTDFDSQ TQVSQSKDSD VYITDKCVLD
MRSMDFKSNS AVAWSQKSDF ACANAFQNSI IPEDT

TCR “a19b21” beta chain sequence (SEQ ID NO: 81), comprising the b21 variable domain (SEQ ID NO: 76—regular text) and a mutated constant domain (SEQ ID NO: 39—italics):

NAGVTQTPKF RILKIGQSMT LQCAQDLQHS YMYWYRQDPG
MGLKPIYYSV GVGFTDKGEV PEGYQVSRST TEDFPLRLES
AAPSQTSVYF CASAYMTGEL FFGEGSRLTV LEDLKNVFPP
EVAVFEPSEA EISHTQKATL VCLATGFYPD HVELSWWVNG
KEVHSGVCTD PQPLKEQPALQD SRYALSSRLR VSATFWQDPR

a19b22 TCR

TCR “a19b22” alpha chain sequence (SEQ ID NO: 40), comprising the a19 variable domain (SEQ ID NO: 26—regular text) described above and a mutated constant domain (SEQ ID NO: 37—italics):

AKEVEQNSGP LSVPEGAIAS LQCTYSDKHS QGFFWYRQYS
GKSPELIMSI YSQGDKEDGR FTAQLNKASQ YVSLLIRDSQ
PSDSATYLCA VRGNEKLTFG TGTRLTIIPN IQNPDPAVYQ
LRDSKSSDKS VCLFTDFDSQ TQVSQSKDSD VYITDKCVLD
MRSMDFKSNS AVAWSQKSDF ACANAFQNSI IPEDT

TCR “a19b22” beta chain sequence (SEQ ID NO: 82), comprising the b22 variable domain (SEQ ID NO: 78—regular text) and a mutated constant domain (SEQ ID NO: 39—italics):

NAGVTQTPKF RILKIGQSMT LQCAQDLQHS YMYWYRQDPG
MGLKPIYYSV GVGFTDKGEV PDGYQVSRST TEDFPLRLES
AAPSQTSVYF CASAYMTGEL FFGEGSRLTV LEDLKNVFPP
EVAVFEPSEA EISHTQKATL VCLATGFYPD HVELSWWVNG
KEVHSGVCTD PQPLKEQPALQD SRYALSSRLR VSATFWQDPR
NHFRCQVQFY GLSENDEWTQ DRAKPVTQIV SAEAWGRAD

### Exemplary PD1 Agonist VHH Sequences

SEQ ID NO: 42 is the amino acid sequence of an exemplary camelid PD1 agonist VHH. The CDRs (CDR1, CDR2 and CDR3) are underlined and are designated SEQ ID NO: 43, 44 and 45 respectively. Positions different from the human IgHV3-23 consensus are shown in bold.

SEQ ID NO: 42:
EVQLVESGGA LVQPGGSLRL SCAASGFTFS SYAMTWVRQA
PGKGPEWVSA IASDGASTSY LDSVKGRFTV SRDNAKNTLY
LQMNSLKPED TAVYYCARGG YLTYDRYGQG TQVTVSS

SEQ ID NO: 71 is the amino acid sequence of a humanised version of the exemplary PD1 agonist VHH of SEQ ID NO: 42. Mutations relative to SEQ ID NO: 42 are shown in bold.

AVQLVESGGG LVQPGGSLRL SCAASGFTFS SYAMTWVRQA
PGKGPEWVSA IASDGASTSY ADSVKGRFTI SRDNSKNTLY
LQMNSLRPED TAVYYCARGG YLTYDRYGQG TLVTVSS

### Exemplary TCR-PD1 Agonist VHH Sequences

a2b3VHH

“a2b3VH” is a binding molecule comprising the TCR “a2” alpha chain (SEQ ID NO: 70) described above and a TCR beta chain-PD1 agonist VHH fusion (SEQ ID NO: 72). The beta chain-PD1 agonist VHH fusion sequence (SEQ ID NO: 72) is shown below and comprises the PD1 agonist VHH of SEQ ID NO: 71, italics) described above fused to the TCR “b3” beta chain (SEQ ID NO: 69) described above. The TCR beta chain and PD1 agonist VHH sequences are linked via a glycine-serine linker (underlined), designated SEQ ID NO: 73.

SEQ ID NO: 72:
AVQLVESGGG LVQPGGSLRL SCAASGFTFS SYAMTWVRQA
PGKGPEWVSA IASDGASTSY ADSVKGRFTI SRDNSKNTLY
LQMNSLRPED TAVYYCARGG YLTYDRYGQG TLVTVSSGGG
GSNAGVTQTP KFRILKIGQS MTLQCAQDMQ HSYMYWYRQD
PGMGLKPIYY SVGVGFTDKG EVPNGYQVSR STTEDFPLRL
ESAAPSQTSV YFCASAYMTG ELFFGEGSRL TVLEDLKNVF
PPEVAVFEPS EAEISHTQKA TLVCLATGFY PDHVELSWWV
NGKEVHSGVC TDPQPLKEQP ALQDSRYALS SRLRVSATFW
QDPRNHFRCQ VQFYGLSEND EWTQDRAKPV TQIVSAEAWG
RAD

a18b16VHH

“a18b16VH” is a binding molecule comprising the TCR “a18” alpha chain (SEQ ID NO: 36) described above and a TCR beta chain-PD1 agonist VHH fusion (SEQ ID NO: 46). The beta chain-PD1 agonist VHH fusion sequence (SEQ ID NO: 46) is shown below and comprises the PD1 agonist VHH of SEQ ID NO: 71, italics) described above fused to the TCR “b16” beta chain (SEQ ID NO: 38) described above. The TCR beta chain and PD1 agonist VHH sequences are linked via a glycine-serine linker (underlined), designated SEQ ID NO: 73.

SEQ ID NO: 46:
AVQLVESGGG LVQPGGSLRL SCAASGFTFS SYAMTWVRQA
PGKGPEWVSA IASDGASTSY ADSVKGRFTI SRDNSKNTLY
LQMNSLRPED TAVYYCARGG YLTYDRYGQG TLVTVSSGGG
GSNAGVTQTP KFRILKIGQS MTLQCAQDMQ HSYMYWYRQD
PGMGLKPIYY SVGVGFTDKG EVPNGYQVSR STTEDFPLRL
ESAAPSQTSV YFCASAYMTG ELFFGEGSRL TVLEDLKNVF
PPEVAVFEPS EAEISHTQKA TLVCLATGFY PDHVELSWWV
NGKEVHSGVC TDPQPLKEQP ALQDSRYALS SRLRVSATFW
QDPRNHFRCQ VQFYGLSEND EWTQDRAKPV TQIVSAEAWG
RAD

a19b19VHH

“a19b19VHH” is a binding molecule comprising the TCR “a19” alpha chain (SEQ ID NO: 40) described above and a TCR beta chain-PD1 agonist VHH fusion (SEQ ID NO: 47). The beta chain-PD1 agonist VHH fusion sequence (SEQ ID NO: 47) is shown below and comprises the PD1 agonist VHH of SEQ ID NO: 71, italics) described above fused to the TCR “b19” beta chain (SEQ ID NO: 41) described above. The TCR beta chain and PD1 agonist VHH sequences are linked via a glycine-serine linker (underlined), designated SEQ ID NO: 73.

SEQ ID NO: 47:
AVQLVESGGG LVQPGGSLRL SCAASGFTFS SYAMTWVRQA
PGKGPEWVSA IASDGASTSY ADSVKGRFTI SRDNSKNTLY
LQMNSLRPED TAVYYCARGG YLTYDRYGQG TLVTVSSGGG
GSNAGVTQTP KFRILKIGQS MTLQCAQDLQ HSYMYWYRQD
PGMGLKPIYY SVGVGFTDKG EVPNGYQVSR STTEDFPLRL
ESAAPSQTSV YFCASAYMTG ELFFGEGSRL TVLEDLKNVF
PPEVAVFEPS EAEISHTQKA TLVCLATGFY PDHVELSWWV
NGKEVHSGVC TDPQPLKEQP ALQDSRYALS SRLRVSATFW
QDPRNHERCQ VQFYGLSEND EWTQDRAKPV TQIVSAEAWG
RAD

a19b20VHH

“a19b20VHH” is a binding molecule comprising the TCR “a19” alpha chain (SEQ ID NO: 40) described above and a TCR beta chain-PD1 agonist VHH fusion (SEQ ID NO: 83). The beta chain-PD1 agonist VHH fusion sequence (SEQ ID NO: 83) is shown below and comprises the PD1 agonist VHH of SEQ ID NO: 71, italics) described above fused to the TCR “b20” beta chain (SEQ ID NO: 80) described above. The TCR beta chain and PD1 agonist VHH sequences are linked via a glycine-serine linker (underlined), designated SEQ ID NO: 73.

SEQ ID NO: 83:
AVQLVESGGG LVQPGGSLRL SCAASGFTFS SYAMTWVRQA
PGKGPEWVSA IASDGASTSY ADSVKGRFTI SRDNSKNTLY
LQMNSLRPED TAVYYCARGG YLTYDRYGQG TLVTVSSGGG
GSNAGVTQTP KFRILKIGQS MTLQCAQDLQ HSYMYWYRQD
PGMGLKPIYY SVGVGFTDKG EVPQGYQVSR STTEDFPLRL
ESAAPSQTSV YFCASAYMTG ELFFGEGSRL TVLEDLKNVF
PPEVAVFEPS EAEISHTQKA TLVCLATGFY PDHVELSWWV
NGKEVHSGVC TDPQPLKEQP ALQDSRYALS SRLRVSATFW
QDPRNHFRCQ VQFYGLSEND EWTQDRAKPV TQIVSAEAWG
RAD

a19b21VHH

“a19b21VHH” is a binding molecule comprising the TCR “a19” alpha chain (SEQ ID NO: 40) described above and a TCR beta chain-PD1 agonist VHH fusion (SEQ ID NO: 84). The beta chain-PD1 agonist VHH fusion sequence (SEQ ID NO: 84) is shown below and comprises the PD1 agonist VHH of SEQ ID NO: 71, italics) described above fused to the TCR “b21” beta chain (SEQ ID NO: 81) described above. The TCR beta chain and PD1 agonist VHH sequences are linked via a glycine-serine linker (underlined), designated SEQ ID NO: 73.

SEQ ID NO: 84:
AVQLVESGGG LVQPGGSLRL SCAASGFTFS SYAMTWVRQA
PGKGPEWVSA IASDGASTSY ADSVKGRFTI SRDNSKNTLY
LQMNSLRPED TAVYYCARGG YLTYDRYGQG TLVTVSSGGG
GSNAGVTQTP KFRILKIGQS MTLQCAQDLQ HSYMYWYRQD
PGMGLKPIYY SVGVGFTDKG EVPEGYQVSR STTEDFPLRL
ESAAPSQTSV YFCASAYMTG ELFFGEGSRL TVLEDLKNVF
PPEVAVFEPS EAEISHTQKA TLVCLATGFY PDHVELSWWV
NGKEVHSGVC TDPQPLKEQP ALQDSRYALS SRLRVSATFW
QDPRNHFRCQ VQFYGLSEND EWTQDRAKPV TQIVSAEAWG
RAD

a19b22VHH

“a19b22VHH” is a binding molecule comprising the TCR “a19” alpha chain (SEQ ID NO: 40) described above and a TCR beta chain-PD1 agonist VHH fusion (SEQ ID NO: 85). The beta chain-PD1 agonist VHH fusion sequence (SEQ ID NO: 85) is shown below and comprises the PD1 agonist VHH of SEQ ID NO: 71, italics) described above fused to the TCR “b22” beta chain (SEQ ID NO: 82) described above. The TCR beta chain and PD1 agonist VHH sequences are linked via a glycine-serine linker (underlined), designated SEQ ID NO: 73.

SEQ ID NO: 85:
AVQLVESGGG LVQPGGSLRL SCAASGFTFS SYAMTWVRQA
PGKGPEWVSA IASDGASTSY ADSVKGRFTI SRDNSKNTLY
LQMNSLRPED TAVYYCARGG YLTYDRYGQG TLVTVSSGGG
GSNAGVTQTP KFRILKIGQS MTLQCAQDLQ HSYMYWYRQD
PGMGLKPIYY SVGVGFTDKG EVPDGYQVSR STTEDEPLRL
ESAAPSQTSV YFCASAYMTG ELFFGEGSRL TVLEDLKNVF
PPEVAVFEPS EAEISHTQKA TLVCLATGFY PDHVELSWWV
NGKEVHSGVC TDPQPLKEQP ALQDSRYALS SRLRVSATFW
QDPRNHFRCQ VQFYGLSEND EWTQDRAKPV TQIVSAEAWG
RAD

### Exemplary Extended Half Life TCR-PD1 Agonist VHH Sequences

a2b3VHH-HLE

“a2b3VHH-HLE” is a binding molecule comprising a first polypeptide chain comprising the TCR beta chain-PD1 agonist of SEQ ID NO: 72 described above, a second polypeptide chain (SEQ ID NO: 48) which comprises the TCR “a2” alpha chain (SEQ ID NO: 70) described above fused to an Fc domain (SEQ ID NO: 49—italics) via a hinge (SEQ ID NO: 50-underlined), and a third polypeptide chain (SEQ ID NO: 51) comprising an Fc domain (SEQ ID NO: 52—italics) fused to a hinge (SEQ ID 50—underlined).

SEQ ID NO: 48:
AKEVEQNSGP LSVPEGAIAS LQCTYSDKHS QGFFWYRQYS
GKSPELIMSI YSNGDKEDGR FTAQLNKASQ YVSLLIRDSQ
PSDSATYLCA VRGNEKLTFG TGTRLTIIPN IQNPDPAVYQ
LRDSKSSDKS VCLFTDFDSQ TQVSQSKDSD VYITDKCVLD
MRSMDFKSNS AVAWSQKSDF ACANAFQNSI IPEDTDKTHT
CPPCPAPELL GGPSVFLFPP KPKDTLMISR TPEVTCVVVD
VSHEDPEVKF NWYVDGVEVH NAKTKPREEQ YGSTYRVVSV
LTVLHQDWLN GKEYKCKVSN KALPAPIEKT ISKAKGQPRE
PQVYTLPPSR DELTKNQVSL SCAVKGFYPS DIAVEWESNG
QPENNYKTTP PVLDSDGSFF LVSKLTVDKS RWQQGNVFSC
SVMHEALHNH YTQKSLSLSPGK
SEQ ID NO: 51:
DKTHTCPPCP APELLGGPSV FLFPPKPKDT LMISRTPEVT
CVVVDVSHED PEVKENWYVD GVEVHNAKTK PREEQYGSTY
RVVSVLTVLH QDWLNGKEYK CKVSNKALPA PIEKTISKAK
GQPREPQVYT LPPSRDELTK NQVSLWCLVK GFYPSDIAVE
WESNGQPENN YKTTPPVLDS DGSFFLYSKL TVDKSRWQQG
NVFSCSVMHE ALHNHYTQKS LSLSPGK

a2b3VHH-HLE (YTE)

“a2b3VHH-HLE (YTE)” is a binding molecule which is identical to “a2b3VHH-HLE” above, except that the Fc region sequences comprise M252Y, S254T and T256E substitutions (EU numbering scheme) to enhance binding to FcRn. a2b3VHH-HLE (YTE) comprises a first polypeptide chain comprising the TCR beta chain-PD1 agonist of SEQ ID NO: 72 described above, a second polypeptide chain (SEQ ID NO: 97) which comprises the TCR “a2” alpha chain (SEQ ID NO: 70) described above fused to an Fc domain (SEQ ID NO: 93—italics) via a hinge (SEQ ID NO: 50—underlined), and a third polypeptide chain (SEQ ID NO: 98) comprising an Fc domain (SEQ ID NO: 94—italics) fused to a hinge (SEQ ID 50—underlined).

SEQ ID NO: 97:
AKEVEQNSGP LSVPEGAIAS LQCTYSDKHS QGFFWYRQYS
GKSPELIMSI YSNGDKEDGR FTAQLNKASQ YVSLLIRDSQ
PSDSATYLCA VRGNEKLTFG TGTRLTIIPN IQNPDPAVYQ
LRDSKSSDKS VCLFTDFDSQ TQVSQSKDSD VYITDKCVLD
MRSMDFKSNS AVAWSQKSDF ACANAFQNSI IPEDTDKTHT
CPPCPAPELL GGPSVFLFPP KPKDTLYITR EPEVTCVVVD
VSHEDPEVKF NWYVDGVEVH NAKTKPREEQ YGSTYRVVSV
LTVLHQDWLN GKEYKCKVSN KALPAPIEKT ISKAKGQPRE
PQVYTLPPSR DELTKNQVSL SCAVKGFYPS DIAVEWESNG
QPENNYKTTP PVLDSDGSFF LVSKLTVDKS RWQQGNVFSC
SVMHEALHNH YTQKSLSLSP GK
SEQ ID NO: 98:
DKTHTCPPCP APELLGGPSV FLFPPKPKDT LYITREPEVT
CVVVDVSHED PEVKENWYVD GVEVHNAKTK PREEQYGSTY
RVVSVLTVLH QDWLNGKEYK CKVSNKALPA PIEKTISKAK
GQPREPQVYT LPPSRDELTK NQVSLWCLVK GFYPSDIAVE
WESNGQPENN YKTTPPVLDS DGSFFLYSKL TVDKSRWOQG
NVFSCSVMHE ALHNHYTQKS LSLSPGK

a18b16VHH-HLE

“a18b16VHH-HLE” is a binding molecule comprising a first polypeptide chain comprising the TCR beta chain-PD1 agonist of SEQ ID NO: 46 described above, a second polypeptide chain (SEQ ID NO: 53) which comprises the TCR “a18” alpha chain (SEQ ID NO: 36) described above fused to an Fc domain (SEQ ID NO: 49—italics) via a hinge (SEQ ID NO: 50-underlined), and a polypeptide third chain (SEQ ID NO: 51) described above comprising an Fc domain (SEQ ID NO: 52) fused to a hinge (SEQ ID 50).

SEQ ID NO: 53:
AKEVEQNSGP LSVPEGAIAS LQCTYSDKHS QGFFWYRQYS
GKSPELIMSI YSNGDKEDGR FTAQLNKASQ YVSLLIRDSQ
PSDSATYLCA VRGNEKLTFG TGTRLTIIPN IQNPDPAVYQ
LRDSKSSDKS VCLFTDFDSQ TQVSQSKDSD VYITDKCVLD
MRSMDFKSNS AVAWSQKSDF ACANAFQNSI IPEDTDKTHT
CPPCPAPELL GGPSVFLFPP KPKDTLMISR TPEVTCVVVD
VSHEDPEVKF NWYVDGVEVH NAKTKPREEQ YGSTYRVVSV
LTVLHQDWLN GKEYKCKVSN KALPAPIEKT ISKAKGQPRE
PQVYTLPPSR DELTKNQVSL SCAVKGFYPS DIAVEWESNG
QPENNYKTTP PVLDSDGSFF LVSKLTVDKS RWQQGNVFSC
SVMHEALHNH YTQKSLSLSP GK

a18b16VHH-HLE (YTE)

“a18b16VHH-HLE (YTE)” is a binding molecule which is identical to “a18b16VHH-HLE” above, except that the Fc region sequences comprise M252Y, S254T and T256E substitutions (EU numbering scheme) to enhance binding to FcRn. a18b16VHH-HLE (YTE) comprises a first polypeptide chain comprising the TCR beta chain-PD1 agonist of SEQ ID NO: 46 described above, a second polypeptide chain (SEQ ID NO: 99) which comprises the TCR “a18” alpha chain (SEQ ID NO: 36) described above fused to an Fc domain (SEQ ID NO: 93—italics) via a hinge (SEQ ID NO: 50-underlined), and a polypeptide third chain (SEQ ID NO: 98) described above comprising an Fc domain (SEQ ID NO: 94) fused to a hinge (SEQ ID 50).

SEQ ID NO: 99:
AKEVEQNSGP LSVPEGAIAS LQCTYSDKHS QGFFWYRQYS
GKSPELIMSI YSNGDKEDGR FTAQLNKASQ YVSLLIRDSQ
PSDSATYLCA VRGNEKLTFG TGTRLTIIPN IQNPDPAVYQ
LRDSKSSDKS VCLFTDFDSQ TQVSQSKDSD VYITDKCVLD
MRSMDFKSNS AVAWSQKSDF ACANAFQNSI IPEDTDKTHT
CPPCPAPELL GGPSVFLFPP KPKDTLYITR EPEVTCVVVD
VSHEDPEVKF NWYVDGVEVH NAKTKPREEQ YGSTYRVVSV
LTVLHQDWLN GKEYKCKVSN KALPAPIEKT ISKAKGQPRE
PQVYTLPPSR DELTKNQVSL SCAVKGFYPS DIAVEWESNG
QPENNYKTTP PVLDSDGSFF LVSKLTVDKS RWQQGNVFSC
SVMHEALHNH YTQKSLSLSP GK

a19b19VHH-HLE

“a19b19VHH-HLE” is a binding molecule comprising a first polypeptide chain comprising the TCR beta chain-PD1 agonist of SEQ ID NO: 47 described above, a second polypeptide chain (SEQ ID NO: 54) which comprises the TCR “a19” alpha chain (SEQ ID NO: 40) described above fused to an Fc domain (SEQ ID NO: 49—italics) via a hinge (SEQ ID NO: 50-underlined), and a third polypeptide chain (SEQ ID NO: 51) described above comprising an Fc domain (SEQ ID NO: 52) fused to a hinge (SEQ ID 50).

SEQ ID NO: 54:
AKEVEQNSGP LSVPEGAIAS LQCTYSDKHS QGFFWYRQYS
GKSPELIMSI YSQGDKEDGR FTAQLNKASQ YVSLLIRDSQ
PSDSATYLCA VRGNEKLTFG TGTRLTIIPN IQNPDPAVYQ
LRDSKSSDKS VCLFTDFDSQ TQVSQSKDSD VYITDKCVLD
MRSMDFKSNS AVAWSQKSDF ACANAFQNSI IPEDTDKTHT
CPPCPAPELL GGPSVFLFPP KPKDTLMISR TPEVTCVVVD
VSHEDPEVKF NWYVDGVEVH NAKTKPREEQ YGSTYRVVSV
LTVLHQDWLN GKEYKCKVSN KALPAPIEKT ISKAKGQPRE
PQVYTLPPSR DELTKNQVSL SCAVKGFYPS DIAVEWESNG
QPENNYKTTP PVLDSDGSFF LVSKLTVDKS RWQQGNVFSC
SVMHEALHNH YTQKSLSLSP GK

a19b19VHH-HLE (YTE)

“a19b19VHH-HLE (YTE)” is a binding molecule which is identical to “a19b19VHH-HLE” above, except that the Fc region sequences comprise M252Y, S254T and T256E substitutions (EU numbering scheme) to enhance binding to FcRn. a19b19VHH-HLE (YTE) comprises a first polypeptide chain comprising the TCR beta chain-PD1 agonist of SEQ ID NO: 47 described above, a second polypeptide chain (SEQ ID NO: 100) which comprises the TCR “a19” alpha chain (SEQ ID NO: 40) described above fused to an Fc domain (SEQ ID NO: 93—italics) via a hinge (SEQ ID NO: 50-underlined), and a third polypeptide chain (SEQ ID NO: 98) described above comprising an Fc domain (SEQ ID NO: 94) fused to a hinge (SEQ ID 50).

SEQ ID NO: 100:
AKEVEQNSGP LSVPEGAIAS LQCTYSDKHS QGFFWYRQYS
GKSPELIMSI YSQGDKEDGR FTAQLNKASQ YVSLLIRDSQ
PSDSATYLCA VRGNEKLTFG TGTRLTIIPN IQNPDPAVYQ
LRDSKSSDKS VCLFTDFDSQ TQVSQSKDSD VYITDKCVLD
MRSMDFKSNS AVAWSQKSDF ACANAFQNSI IPEDTDKTHT
CPPCPAPELL GGPSVFLFPP KPKDTLYITR EPEVTCVVVD
VSHEDPEVKF NWYVDGVEVH NAKTKPREEQ YGSTYRVVSV
LTVLHQDWLN GKEYKCKVSN KALPAPIEKT ISKAKGQPRE
PQVYTLPPSR DELTKNQVSL SCAVKGFYPS DIAVEWESNG
QPENNYKTTP PVLDSDGSFF LVSKLTVDKS RWQQGNVFSC
SVMHEALHNH YTQKSLSLSP GK

a19b20VHH-HLE

“a19b20VHH-HLE” is a binding molecule comprising a first polypeptide chain comprising the TCR beta chain-PD1 agonist of SEQ ID NO: 83 described above, a second polypeptide chain of SEQ ID NO: 54, described above, comprising the TCR “a19” alpha chain (SEQ ID NO: 40) described above fused to an Fc domain (SEQ ID NO: 49) via a hinge (SEQ ID NO: 50), and a third polypeptide chain (SEQ ID NO: 51) described above comprising an Fc domain (SEQ ID NO: 52) fused to a hinge (SEQ ID 50).

a19b20VHH-HLE (YTE)

“a19b20VHH-HLE (YTE)” is a binding molecule which is identical to “a19b20VHH-HLE” above, except that the Fc region sequences comprise M252Y, S254T and T256E substitutions (EU numbering scheme) to enhance binding to FcRn. a19b20VHH-HLE (YTE) comprises a first polypeptide chain comprising the TCR beta chain-PD1 agonist of SEQ ID NO: 83 described above, a second polypeptide chain of SEQ ID NO: 100, described above, comprising the TCR “a19” alpha chain (SEQ ID NO: 40) described above fused to an Fc domain (SEQ ID NO: 93) via a hinge (SEQ ID NO: 50), and a third polypeptide chain (SEQ ID NO: 98) described above comprising an Fc domain (SEQ ID NO: 94) fused to a hinge (SEQ ID 50).

a19b21VHH-HLE

“a19b21VHH-HL” is a binding molecule comprising a first polypeptide chain comprising the TCR beta chain-PD1 agonist of SEQ ID NO: 84 described above, a second polypeptide chain of SEQ ID NO: 54, described above, comprising the TCR “a19” alpha chain (SEQ ID NO: 40) described above fused to an Fc domain (SEQ ID NO: 49) via a hinge (SEQ ID NO: 50), and a third polypeptide chain (SEQ ID NO: 51) described above comprising an Fc domain (SEQ ID NO: 52) fused to a hinge (SEQ ID 50).

a19b21VHH-HLE (YTE)

“a19b21VHH-HLE (YTE)” is a binding molecule which is identical to “a19b21VHH-HLE” above, except that the Fc region sequences comprise M252Y, S254T and T256E substitutions (EU numbering scheme) to enhance binding to FcRn. a19b21VHH-HLE (YTE) comprises a first polypeptide chain comprising the TCR beta chain-PD1 agonist of SEQ ID NO: 84 described above, a second polypeptide chain of SEQ ID NO: 100, described above, comprising the TCR “a19” alpha chain (SEQ ID NO: 40) described above fused to an Fc domain (SEQ ID NO: 93) via a hinge (SEQ ID NO: 50), and a third polypeptide chain (SEQ ID NO: 98) described above comprising an Fc domain (SEQ ID NO: 94) fused to a hinge (SEQ ID 50).

a19b22VHH-HLE

“a19b22VHH-HL” is a binding molecule comprising a first polypeptide chain comprising the TCR beta chain-PD1 agonist of SEQ ID NO: 85 described above, a second polypeptide chain of SEQ ID NO: 54, described above, comprising the TCR “a19” alpha chain (SEQ ID NO: 40) described above fused to an Fc domain (SEQ ID NO: 49) via a hinge (SEQ ID NO: 50), and a third polypeptide chain (SEQ ID NO: 51) described above comprising an Fc domain (SEQ ID NO: 52) fused to a hinge (SEQ ID 50).

a19b22VHH-HLE (YTE)

“a19b22VHH-HLE (YTE)” is a binding molecule which is identical to “a19b22VHH-HLE” above, except that the Fc region sequences comprise M252Y, S254T and T256E substitutions (EU numbering scheme) to enhance binding to FcRn. a19b22VHH-HLE (YTE) comprises a first polypeptide chain comprising the TCR beta chain-PD1 agonist of SEQ ID NO: 85 described above, a second polypeptide chain of SEQ ID NO: 100, described above, comprising the TCR “a19” alpha chain (SEQ ID NO: 40) described above fused to an Fc domain (SEQ ID NO: 93) via a hinge (SEQ ID NO: 50), and a third polypeptide chain (SEQ ID NO: 98) described above comprising an Fc domain (SEQ ID NO: 94) fused to a hinge (SEQ ID 50).

### Exemplary Fc Domain Sequences

Human IgG1 Fc region (CH2 and CH3 domains), unmodified (SEQ ID NO: 92):

APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK

Another exemplary IgG1 Fc region sequence is shown below (SEQ ID NO: 49). This sequence has four substitutions, (bold), relative to the above unmodified IgG1 Fc sequence (SEQ ID NO: 92). These are an N297G substitution for inhibiting binding to FcγR as well as T366S, L368A, and Y407V substitutions (hole-forming substitutions) for enhancing dimerization with another Fc region (e.g., SEQ ID NO: 52) containing a T366W substitution (knob-forming substitution). The numbering of the substitutions in this sequence is according to the EU numbering scheme.

(SEQ ID NO: 49)
APELLGGP SVFLFPPKPK DTLMISRTPE VTCVVVDVSH
EDPEVKFNWY VDGVEVHNAK TKPREEQYGS TYRVVSVLTV
LHQDWLNGKE YKCKVSNKAL PAPIEKTISK AKGQPREPQV
YTLPPSRDEL TKNQVSLSCA VKGFYPSDIA VEWESNGQPE
NNYKTTPPVL DSDGSFFLVS KLTVDKSRWQ QGNVFSCSVM
HEALHNHYTQ KSLSLSPGK

Another exemplary IgG1 Fc region sequence is shown below (SEQ ID NO: 52). This sequence has two substitutions, double underlined, relative to the above unmodified IgG1 Fc sequence (SEQ ID NO: 92). These are an N297G substitution for inhibiting binding to FcγR as well as a T366W substitution (knob-forming substitution) for enhancing dimerization with another Fc region (e.g., SEQ ID NO: 49) containing T366S, L368A, and Y407V substitutions (hole-forming substitutions). The numbering of the substitutions in this sequence is according to the EU numbering scheme.

(SEQ ID NO: 52)
APELLGGP SVFLFPPKPK DTLMISRTPE VTCVVVDVSH
EDPEVKFNWY VDGVEVHNAK TKPREEQYGS TYRVVSVLTV
LHQDWLNGKE YKCKVSNKAL PAPIEKTISK AKGQPREPQV
YTLPPSRDEL TKNQVSLWCL VKGFYPSDIA VEWESNGQPE
NNYKTTPPVL DSDGSFFLYS KLTVDKSRWQ QGNVFSCSVM
HEALHNHYTQ KSLSLSPGK

Another exemplary IgG1 Fc region sequence is shown below (SEQ ID NO: 93). This sequence is identical to SEQ ID NO: 49 above except that it has additional M252Y, S254T and T256E substitutions (EU numbering scheme), shown in bold, to enhance binding to FcRn.

(SEQ ID NO: 93)
APELLGGP SVFLFPPKPK DTLYITREPE VTCVVVDVSH
EDPEVKFNWY VDGVEVHNAK TKPREEQYGS TYRVVSVLTV
LHQDWLNGKE YKCKVSNKAL PAPIEKTISK AKGQPREPQV
YTLPPSRDEL TKNQVSLSCA VKGFYPSDIA VEWESNGQPE
NNYKTTPPVL DSDGSFFLVS KLTVDKSRWQ QGNVFSCSVM
HEALHNHYTQ KSLSLSPGK

Another exemplary IgG1 Fc region sequence is shown below (SEQ ID NO: 94). This sequence is identical to SEQ ID NO: 52 above except that it has additional M252Y, S254T and T256E substitutions (EU numbering scheme), shown in bold, to enhance binding to FcRn.

(SEQ ID NO: 94)
APELLGGP SVFLFPPKPK DTLYITREPE VTCVVVDVSH
EDPEVKFNWY VDGVEVHNAK TKPREEQYGS TYRVVSVLTV
LHQDWLNGKE YKCKVSNKAL PAPIEKTISK AKGQPREPQV
YTLPPSRDEL TKNQVSLWCL VKGFYPSDIA VEWESNGQPE
NNYKTTPPVL DSDGSFFLYS KLTVDKSRWQ QGNVFSCSVM
HEALHNHYTQ KSLSLSPGK

### Exemplary IgG Hinge Sequences

SEQ ID NO: 95 below is an exemplary IgG1 hinge sequence (containing a C to S substitution at position 5, numbered according to SEQ ID NO: 95, relative to the native human IgG1 sequence; double underlined):

EPKSSDKTHTCPPCP

SEQ ID NO: 50 below is another exemplary IgG1 hinge sequence (which is a shorter version of the above IgG1 hinge sequence):

(SEQ ID NO: 50)
DKTHTCPPCP

SEQ ID NO: 96 below is an exemplary IgG4 hinge sequence

ESKYGPPCPSCP

### Exemplary Amino Acid Linker Sequences

(SEQ ID NO: 73)
GGGGS,
(SEQ ID NO: 55)
GGGSG,
(SEQ ID NO: 56)
GGSGG,
(SEQ ID NO: 57)
GSGGG,
(SEQ ID NO: 58)
GSGGGP,
(SEQ ID NO: 59)
GGEPS,
(SEQ ID NO: 60)
GGEGGGP,
(SEQ ID NO: 61)
GGEGGGSEGGGS,
(SEQ ID NO: 62)
GGGSGGGG,
(SEQ ID NO: 63)
GGGGSGGGGSGGGGSGGGGSGGGS,
(SEQ ID NO: 64)
GGGGSGGGGSGGGGSGGGGS,
(SEQ ID NO: 65)
EAAAK,
and
(SEQ ID NO: 66)
EAAAKEAAAKEAAAK.

Amino Acid Sequences from Human PD-1 and PD-L1

SEQ ID NO: 101 is the amino acid sequence of the extracellular region of human PD-1 (bold residues are in the epitope of the VHH provided in SEQ ID NOs: 42 and 71):

FLDSPDRPWNPPTFSPALLVVTEGDNATFTCSFSNTSESFVLNWYRMSPS
NQTDKLAAFPEDRSQPGQDCRFRVTQLPNGRDFHMSVVRARRNDSGTYLC
GAISLAPKAQIKESLRAELRVTERRAEVPTAHPSPSPRPAGQFQTLV

SEQ ID NO: 102 is the amino acid sequence of a soluble fragment of human PD-L1 that is capable of binding to PD-1:

FTVTVPKDLYWEYGSNMTIECKFPVEKQLDLAALIVYWEMEDKNIIQFVH
GEEDLKVQHSSYRQRARLLKDQLSLGNAALQITDVKLQDAGVYRCMISYG
GADYKRITVKVNAPY

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the purview of this application and scope of the appended claims.

### Example 1: Isolation of Soluble Native TCRs that Bind to a HLA-A*02 Restricted Peptide from PreProInsulin (PPI) and not to Mimetic Peptides

a) Production of soluble native TCRs

TCRs that bind to the HLA-A*02 restricted peptide ALWGPDPAAA, derived from PreProinsulin, were isolated by panning TCR phage libraries, and the amino acid sequences of the corresponding TCR alpha and beta variable regions determined. The construction and panning of native TCR phage libraries has been described previously (WO2015136072, WO2017046201, WO2017046198). Soluble TCRs were created by fusing the variable regions to truncated versions of the respective alpha and beta chain constant domains, and a non-native interchain disulphide bond was incorporated between constant domain residues as previously described (WO2003020763). To purify soluble TCRs the alpha and beta chains were expressed separately in E. coli inclusion bodies. Solubilised inclusion bodies containing alpha and beta chain were then combined. Refolded soluble TCRs were further purified by anion exchange and size exclusion chromatography using established methods Boulter, et al. (2003), Protein Eng. 16, 707-711; Liddy, et al. (2012), Nature medicine vol. 18,6:980-7). Yield was calculated from the concentration of purified material as determined by absorbance at 280 nm using a Nanodrop spectrophotometer.

### b) Binding Characterisation

To assess the ability of soluble TCRs to recognise the target peptide-MHC complex, binding parameters were obtained by Surface Plasmon Resonance (SPR). SPR measurements were carried out on a BIAcore 8K, BIAcore 3000 or BIAcore T200 instrument. Briefly, biotinylated class I HLA-A*02 molecules were refolded with the peptide of interest and purified using available methods (O'Callaghan et al. (1999), Anal Biochem 266(1):9-15; Garboczi, et al. (1992), Proc Natl Acad Sci USA 89(8):3429-3433). Biotinylated peptide-MHC monomers were immobilized on to streptavidin-coupled CM-5 sensor chips. Equilibrium binding constants were determined using serial dilutions of soluble TCR. K_{D} values were obtained by non-linear curve fitting using Prism software and the Langmuir binding isotherm, bound=C*Max/(C+KD), where “bound” is the equilibrium binding in response units at injected TCR concentration C and Max is the maximum binding. Measurements were performed at 25° C., unless otherwise indicated, in Dulbecco's PBS buffer, supplemented with 0.005% P20.

The table below shows details of two soluble TCRs that were identified from the libraries. Both TCRs bind to the target peptide-MHC complex with an affinity in the low micromolar range and can be purified from E. coli at high yield. The full amino acid sequence of the soluble S2 TCR alpha and beta chains is provided in SEQ ID NO: 2 and SEQ ID NO: 12 respectively

TABLE 1

| | Chain | | | | |
|---|---|---|---|---|---|
| | pairing | | | Binding | |
| | (TRAV/ | | | affinity K_{D} | Yield |
| TCR | TRBV) | aCDR3 | bCDR3 | (μM) | (mg/L) |
| S1 | 12-2/6-2/3 | CAVGAGSARQLTF | CASSQRGTFGEQFF | 11 | 17.2 |
| | | (SEQ ID NO: 86) | (SEQ ID NO: 87) | | |
| S2 | 12-2/6-6 | CAVRGNEKLTF | CASSYMTGELFF | 14 | 14.8 |
| | | (SEQ ID NO: 7) | (SEQ ID NO: 17) | | |

### c) Specificity Assessment

To determine the specificity of target recognition, binding of the native TCRs to alternative peptide-MHC complexes was assessed using the same Biacore procedure described above.

First, peptide mimetics of the target sequence ALWGPDPAAA were identified that have up to three mismatches and have been determined by mass spectrometry to be naturally presented on the cell surface in complex with HLA-A*02. Five mimetic peptides were identified and are shown in the table below.

TABLE 2

| Mimetic | | |
|---|---|---|
| ID | Peptide (SEQ ID) | Protein |
| Mim1 | ALLGPDPAAA (SEQ ID NO: 67) | Basic helix-loop-helix family, member e41 |
| Mim2 | ALPGPDEAAA (SEQ ID NO: 88) | nuclear DEAF-1 related transcriptional |
| | | regulator protein 8 |
| Mim3 | ALMSPPPAAA (SEQ ID NO: 89) | forkhead box protein F2 |
| Mim4 | ALWDPGPEAA (SEQ ID NO: 90) | myeloid zinc finger protein |
| Mim5 | ALGSPPPAAA (SEQ ID NO: 91) | histone-lysine N-methyltransferase 2D |

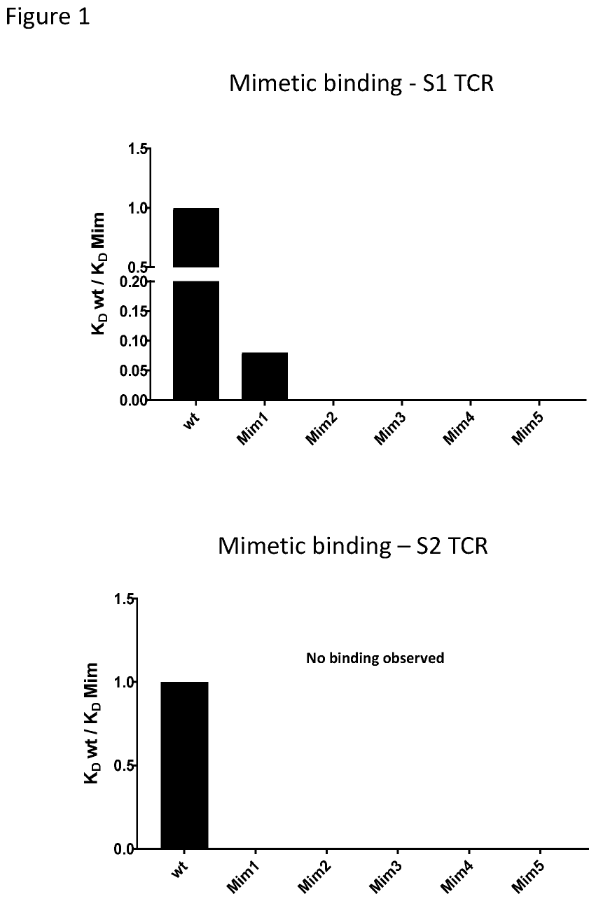

FIG. 1 shows relative binding of the soluble TCRs to each of the mimetic peptides. The data show that TCR S2 had no detectable binding to any of the above mimetic peptides, which are highly similar to the reference peptide ALWGPDPAAA (SEQ ID NO: 1). Whereas for TCR S1 binding was detected to Mim1.

Second, TCR binding was assessed against a mixture containing 20 HLA-A*02 bound peptides that were determined by mass spec to be naturally presented on cells at high abundance. In this case, no binding was detectable for TCR S1 and S2.

### d) Generation of Binding Motif

To further investigate the interaction of the soluble TCRs with the peptide ALWGPDPAAA (SEQ ID NO: 1), the binding motif of each was determined using a similar approach as previously described (WO2014096803). Briefly, each amino acid in the peptide was sequentially replaced with serine and TCR binding assessed by Biacore. Positions in the peptide were considered essential to recognition, and thus part of the binding motif, if the corresponding serine substituted variant resulted in 50% or greater reduction in binding affinity relative to the WT peptide. The greater the number of essential residues in the motif the greater the specificity of the TCR.

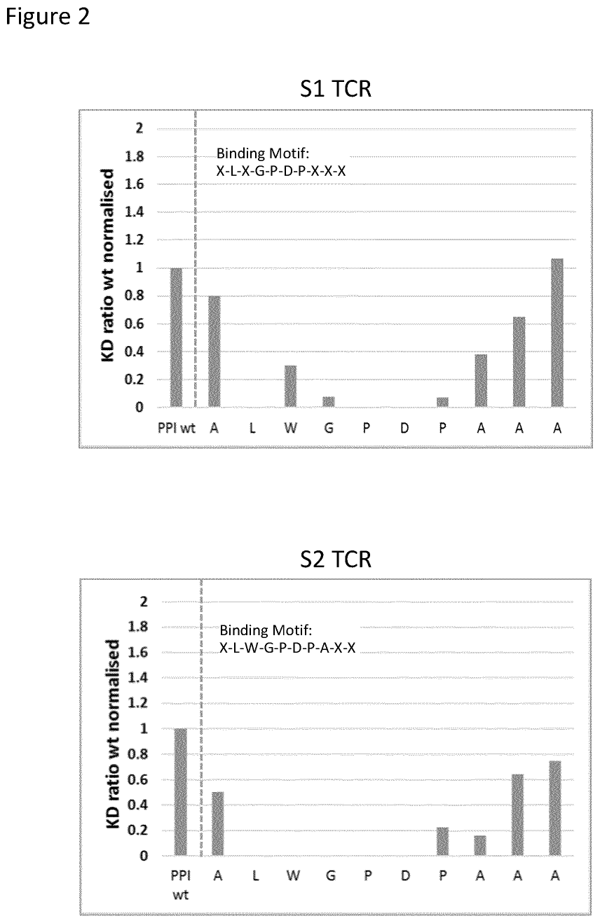

FIG. 2 shows the binding motif for S1 and S2 TCRs. For TCR S1 five residues were shown to be essential for recognition, while for TCR S2 7 residues were essential.

### Example 2: Generation of a Soluble TCR Variant with pM Affinity and High Specificity Following Affinity Maturation

### a) Affinity Maturation by Phage Display

The native soluble S2 TCR was used as a template to identify mutations with higher affinity as previously described (Li et al. (2005), Nat. Biotechnol. 23, 349-354). Briefly, TCR phage libraries were constructed using NNK oligonucleotides to generate mutations in the TCR alpha and beta complementarity-determining regions (CDRs). Typically, several rounds of affinity maturation are required to achieve picomolar (pM) affinity. Mimetic peptide binding was monitored during affinity maturation.

### b) Binding Characterisation

For high affinity interactions binding parameters were determined by single cycle kinetics analysis. Five different concentrations of binding molecule were injected over a flow cell coated with ~100-200 RU (or 50-100 RU for Biacore 8K instrument) of peptide-MHC complex using a flow rate of 50-60 μl min-1. Typically, 60-120 μl (or approx. 240 μl for Biacore 8K instrument) of binding molecule was injected at a top concentration of between 50-100 nM (or 2-50 nM for Biacore 8K instrument), with successive 2 fold dilutions used for the other four injections. The lowest concentration was injected first. To measure the dissociation phase, buffer was injected until ≥10% dissociation occurred, typically after 1-3 hours. Kinetic parameters were calculated using BIAevaluation® software. The dissociation phase was fitted to a single exponential decay equation enabling calculation of half-life. The equilibrium constant K_{D} was calculated from koff/kon. Measurements were performed at 25° C., unless otherwise indicated, in Dulbecco's PBS buffer, supplemented with 0.005% P20.

Mutations giving rise to pM affinity target binding, whist also maintaining a high level of binding specificity were surprisingly identified. The resulting TCR was denoted “a2b3”. Binding to mim1 was detected at the nM range. No binding was detected to any of the other mimetics.

TABLE 3

| | TRAV | TRBV | | | | | Mim1 |
|---|---|---|---|---|---|---|---|
| Molecule | SEQ | SEQ | alpha chain | beta chain | PPI (ALWGPDPAAA) | | (ALLGPDPAAA) |
| ID | ID | ID | (SEQ ID) | (SEQ ID) | K_{D} | T ½ | K_{D} |
| a2b3 | 22 | 68 | 70 | 69 | 15 pM | >20 h | 66 nM |

These data demonstrate that the TCR a2b3 has a high degree of specificity and can sufficiently distinguish a mimetic with just 1 residue mismatch. The affinity window between (SEQ ID NO: 1) and off target (Mim1) recognition was 4400 fold, which provides a large potential safety window and indicates that the TCR is particularly suitable for development as a therapeutic for the treatment of type I diabetes (T1D).

### Example 3: Soluble TCRs Demonstrate Increased Specificity and Greater Yield Relative to Previously Disclosed TCRs that Bind the Same Peptide

Soluble high affinity TCRs that recognise the same ALWGPDPAAA (SEQ ID NO: 1)-HLA-A*02 complex have been disclosed in WO2015092362. The disclosed TCRs are all variants of an alternative native TCR which was isolated from a T cell clone obtained from a human donor. Further analysis of the prior disclosed TCRs was carried out to determine production yield and specificity.

A soluble TCR comprising alpha and beta variable domains corresponding to SEQ ID NOs 58 and 90 in WO2015092362 respectively was assessed using the methods described above in Example 1 and Example 2. The binding affinity of this TCR for ALWGPDPAAA (SEQ ID NO: 1)-HLA-A*02 complex was found to be 240 μM, When expressed in E. coli using the methods described above, this TCR had a yield of <400 μg/L. In addition, the same TCR was shown to bind to Mim1 with an affinity window of <3 fold using the same Biacore methods as those described above. These data indicate that the prior disclosed TCRs in WO2015092362 had substantially lower yield and specificity compared to the TCRs disclosed herein and are therefore not as suitable for the development of therapeutics for T1D.

### Example 4: Binding Molecules Comprising TCR Variants Fused to a PD-1 Agonist Binding Domain Retain Target Specificity and are Functional In Vitro

### a) Sequence Optimisation

The soluble TCR a2b3 was further mutated to remove potential glycosylation sites and modify other residues that were considered to be potentially detrimental to manufacturing processes. The sequences of the resulting soluble TCR variants are provided.

TABLE 4

| Molecule ID | Alpha chain (SEQ ID) | Beta chain (SEQ ID) |
|---|---|---|
| a18b16 | 36 | 38 |
| a19b19 | 40 | 41 |
| a19b20 | 40 | 80 |
| a19b21 | 40 | 81 |
| a19b22 | 40 | 82 |

### b) PD1 Agonist Immune Suppressor

A PD-1 agonist antibody VHH domain was fused to the N terminus of the beta chain of the soluble TCR via a short linker to produce a “TCR PD-1” agonist binding molecule.

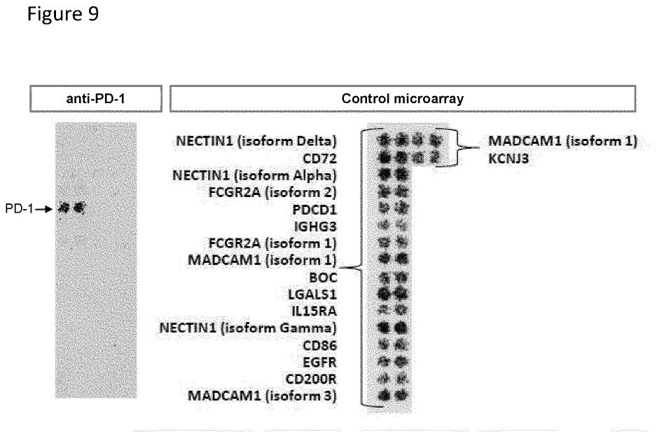

PD-1 specific antibodies were generated by immunisation of llamas with recombinant human PD-1-His protein (Acro Biosystems #PD1-H5221) and after 2-3 rounds of phage-display panning on immobilised recombinant human PD-1-Fc protein (Acro Biosystems #PD1-H5257). Specificity of the selected PD-1 agonist VHH domain in monomeric form was confirmed using Retrogenix Cell Microarray Technology comprising a panel of >5000 cell bound antigens (FIG. 9). Biacore measurements confirmed that TCR PD-1 agonist molecules bind PD-1 in a non-competitive manner with its natural ligand (PD-L1) and therefore are additive to the natural PD-L1 response. Briefly, biotinylated PPI peptide HLA-A*02 complexes were immobilized on a streptavidin-coated CM5 chip. TCR PD-1 agonist molecules were captured onto the chip via the affinity-enhanced PPI₁₅₋₂₄ TCR-PPI₁₅₋₂₄ peptide HLA-A*02 interaction. Excess PD-1 was passed over the chip (1 μM or 10× K_{D} of each PD-1 antibody), followed by an excess of PD-1 and PD-L1-Fc (15 μM). The PD-1 agonist VHH was determined to bind to PD-1 with a K_{D} in the range of 50-70 nM and a binding half life in the range of 15-20 seconds. The sequence of the PD-1 VHH domain is provided in SEQ ID NO: 42 and a humanised variant of the same VHH is provided in SEQ ID NO: 71.

Crystallography was used to map the epitope recognised by the PD-1 agonist VHH. All three CDR loops were shown to mediate contacts to PD-1. Molecule modelling showed that the epitope was located away from the membrane proximal region and is adjacent to but does not overlap the binding site for PD-L1, in line with Biacore competition measurements. The key residues that the PD-1 agonist VHH (SEQ ID NO: 42 and 71) binds to are indicated in bold text in the sequence of the extracellular domain of human PD-1 below (SEQ ID NO:101):

FLDSPDRPWNPPTFSPALLVVTEGDNATFTCSFSNTSESFVLNWYRMSPS
NQTDKLAAFPEDRSQPGQDCRFRVTQLPNGRDFHMSVVRARRNDSGTYLC
GAISLAPKAQIKESLRAELRVTERRAEVPTAHPSPSPRPAGQFQTLV

### c) Half-Life Extending (Fc) Domain

To extend in vivo half-life, a functionally silent Fc domain (comprising SEQ ID NO: 49 and SEQ ID NO: 52) was attached to the C terminus of the TCR alpha chain via a truncated hinge region (SEQ ID NO: 50).

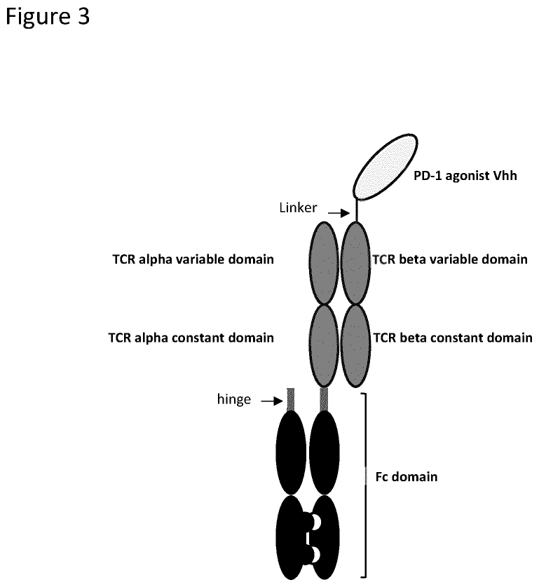

FIG. 3 provides a schematic of the resulting half-life extended TCR PD-1 agonist binding molecule.

### d) Mammalian Expression

Molecules were expressed in CHO cells using the Thermo ExpiCHO™ transient expression protocol, followed by purification using immobilized metal affinity chromatography and size exclusion chromatography.

### e) Biophysical Characterisation

TCR PD-1 agonist binding molecules with an Fc domain were tested for binding to the target peptide and mimetic peptides. Experiments were carried out using single cycle kinetics as described above, except that measurements were performed at 37° C.

TABLE 5

| | | | alpha | beta | | | | | |
|---|---|---|---|---|---|---|---|---|---|
| | TRAV | TRBV | chain | chain | | PPI | | | |
| Molecule | SEQ | SEQ | (SEQ | (SEQ | Yield | (ALWGPDPAAA) | | K_{D} mim1 | Window |
| ID | ID | ID | ID) | ID) | (mg/L) | K_{D} (pM) | T ½ (h) | (nM) | to mim1 |
| a18b16 | 24 | 31 | 36 | 38 | 27 | 39 | 15.6 | 31 | 503 |
| a19b19 | 26 | 34 | 40 | 41 | 39.9 | 57 | 14.0 | 60 | 1052 |
| a19b20 | 26 | 74 | 40 | 80 | 49.5 | 39 | 15.2 | 50 | 1282 |
| a19b21 | 26 | 76 | 40 | 81 | 53.2 | 40 | 15.5 | 65 | 1625 |
| a19b22 | 26 | 78 | 40 | 82 | 43.5 | 40 | 16.7 | 60 | 1500 |

Data show that TCR PD-1 agonist binding molecules including an Fc domain, can be produced in mammalian cells at high yield and maintain high affinity recognition of target and a suitable window of binding to mim1. This high level of specificity indicates that the molecules are particularly suitable for therapeutic development as a potential treatment of T1D.

### f) In Vitro Function-Jurkat NFAT Cell Reporter Assays

To determine the ability of the TCR PD-1 agonist binding molecules to inhibit signalling in activated T cells a NFAT reporter assay was developed. Briefly, Jurkat cells, expressing i) a TCR specific for a HLA-A*02 restricted peptide from Melan A (ELAGIGILTV), ii) PD-1, and iii) a luciferase reporter driven by an NFAT-response element, were incubated with PPI positive beta cell line ECN90 pulsed with a peptide derived from Melan-A to trigger TCR signalling and NFAT promoter-mediated luminescence. Control experiments were performed using PPI negative target cells lines (Mel624, NCI-H1703) in place of ECN90.

Target cells were harvested and plated at 50000 cells/well in Optiβ3 media, into the inner 60 wells of a white 96-well cell culture plate that had been pre-coated with β-coat (Univercell Biosolutions). After incubating at 37° C., 5% CO₂ for 16-20 hours, media was removed and assay buffer containing Melan-A peptide was added. No peptide was added to Mel624 melanoma line that naturally present the melan-A peptide. After pulsing for 2 hours at 37° C., 5% CO₂, assay buffer alone or assay buffer containing titrations of TCR PD-1 agonist binding molecules was added to each well. The assay was initiated by immediately adding 50000 Jurkat NFL Mel5 PD-1 effector cells and incubating for 16-20 hours at 37° C., 5% CO₂. Bioluminescent signal was detected and quantified using Bio-Glo™ Luciferase Assay System (Promega) and a luminometer (CLARIOstar). NFAT activity was normalised against TCR-stimulated controls and dose response data was analyzed in Prism (GraphPad) using a four parameter, non-linear least squares fit to determine IC₅₀ values.

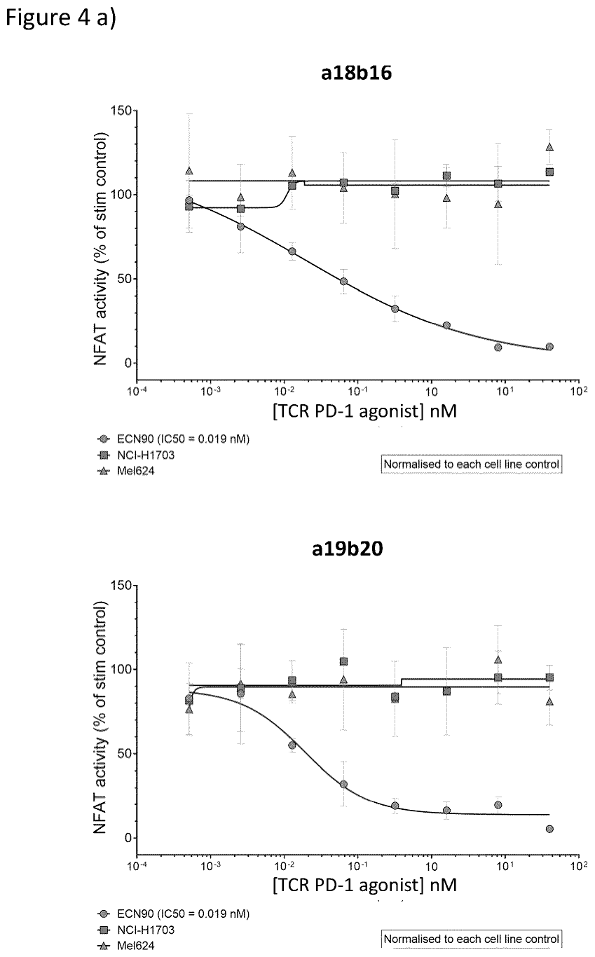

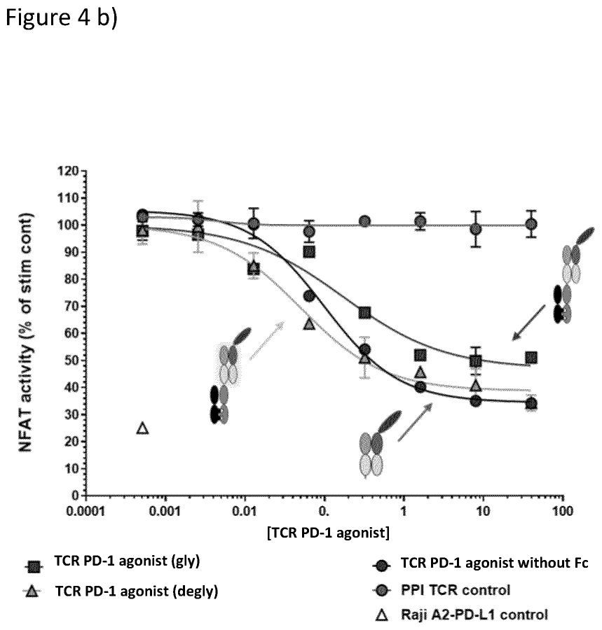

The resulting IC₅₀ values are provided in the table below for each of the indicated TCR-PD1 agonist binding molecules. Values are based on averages from 2 independent experiments. FIG. 4 shows data for 1 experiment from two of the molecules tested.

TABLE 6

| Molecule ID | IC₅₀ (pM) | | Max NFAT inhibition (%) | |
|---|---|---|---|---|
| a18b16 | 24 | (n = 2:19/29) | 89% | (90.25/86.7) |
| a19b19 | 23 | (n = 2:21/25) | 92% | (89.08/85) |
| a19b20 | 23 | (n = 2:19/27) | 92% | (94.62/88.9) |
| a19b21 | 21 | (n = 2:13/29) | 92% | (96.64/86.56) |
| a19b22 | 112.5 | (n = 2:113/112) | 88% | (88.19/87.8) |

A similar NFAT based reporter assay was used to assess the impact of the Fc domain on IC₅₀ values.

In this case HLA-A*02 human B lymphoblastoid cells (Raji) pulsed for 2 hours at 37° C., 5% CO₂ with 20 μM PPI peptide were used as target cells. Cells were harvested and plated at 50000 cells/well in assay media (R10 without antibiotics), into the inner 60 wells of a white 96-well cell culture plate. Subsequently, the cells were treated with 2 μg/ml SEB (Staphylococcal enterotoxin B) for 1 hour at 37° C., 5% CO₂. The assay buffer alone or assay buffer containing titrations of TCR PD-1 agonist binding molecules was added to each well. The assay was initiated by immediately adding 50000 Jurkat NFL Mel5 PD-1 effector cells and incubating for 16-20 hours at 37° C., 5% CO₂. Bioluminescent signal was detected and as described above.

Data were obtained using TCR-PD1 agonist binding molecule a18b16 with an Fc domain as described in Example 4 and a glycosylated variant with or without the Fc domain.

The data in FIG. 4b show that inclusion of the Fc domain has a minimal impact on in vitro potency.

The data from the reporter assays demonstrate that TCR PD-1 agonist binding molecules can potently inhibit activation of T cells and indicate the therapeutic potential of the molecules for the treatment of T1D.

### Example 5: TCR-PD1 Agonist Binding Molecules Provide Extended In Vivo Half Life

Pharmacokinetic properties of the TCR PD-1 agonist binding molecule a18b16 with an Fc domain (as described in example 4) was assessed in SCID mice. Test article was dosed intravenously (IV) or subcutaneously (SC) at 1 mg/Kg, with serial sampling of blood over a 21 day period. Four mice were sampled per time point per dosing route. The binding molecule was detected in serum using a bifunctional MSD (Meso Scale Diagnostics) assay. PK parameters were extracted by non-compartmental analysis.

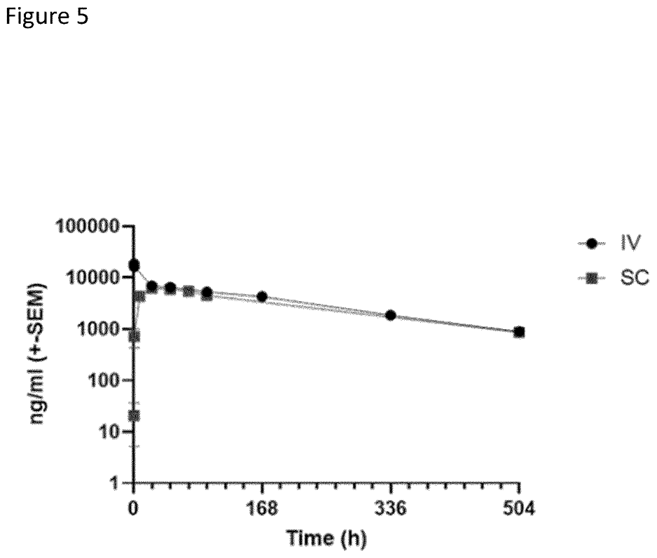

Mean PK parameters are shown in the table below. FIG. 5 shows concentrations of reagent in serum over the course of three weeks.

TABLE 7

| Dose | Tₘₐₓ | Cₘₐₓ | AUCₗₐₛₜ | AUC_{inf} | t_{1/2} | CL ^{a} | V ^{b} |
|---|---|---|---|---|---|---|---|
| Route (N) | (h) | (ng/mL) | (h*ng/mL) | (h*ng/mL) | (h) | (mL/h/kg) | (mL/kg) |
| Intravenous | 0.08 | 18500 | 1760000 | 1970000 | 6.57 | 0.509 | 116 |
| Subcutaneous | 24 | 6210 | 1400000 | 1610000 | 6.89 | 0.621 | 148 |

This study indicated that the TCR PD-1 agonist with Fc has a terminal t_{1/2} of approximately 7 days and a subcutaneous bioavailability of >80%. These properties indicate a therapeutic potential to provide a convenient dosing schedule for the treatment of T1D.

### Example 6-TCR-PD-1 Agonist Binding Molecules Demonstrate Robust Efficacy in In Vitro Models

### a) Primary Human T Cell IL-2 Assay

TCR PD-1 agonist binding molecules as described in Example 4 were tested to determine their ability to inhibit activation of primary human CD4⁺ T cells by antigen-presenting cells (APCs). Free PD-1 agonist was used as a control, along with a non-targeted TCR PD-1 agonist control that does not bind to PPI peptide.

Raji cells transduced with an HLA-A*02 B2-microglobulin were used as APCs (Raji-A2). Primary human CD4⁺ T cells were isolated from PBMCs using a pan T cell isolation kit (Miltenyi). T cells were pre-activated by incubating with irradiated Raji A2 cells, pre-loaded with 1 μg/ml SEB (Sigma). After pre-activation, the expanded T cells were predominantly CD4⁺ T cells and typically 60-70% PD-1 positive Raji A2 cells were pulsed, or not, with 20 μM PPI peptide at 2×10⁶ cell/ml in R10 for 2 hours at 37° C., 5% CO₂. Raji A2 cells were then loaded with 31.6 ng/ml SEB for 1 hour at 37° C., 5% CO₂ and irradiated with 33Gy. Raji A2 cells were plated at 100,000 cells/well and test molecule added. After 1 hour preincubation, washed pre-activated T cells were added to the Raji A2 cells at 100,000 cells/well and incubated for 48 hours at 37° C., 5% CO₂. Supernatants were collected and IL-2 levels were measured by ELISA (IL2 Ready-SET-Go! ELISA, Invitrogen). IL-2 release was normalised against SEB-stimulated controls and dose response data was analyzed in Prism (GraphPad) using a four parameter, non-linear least squares fit to determine IC₅₀ values.

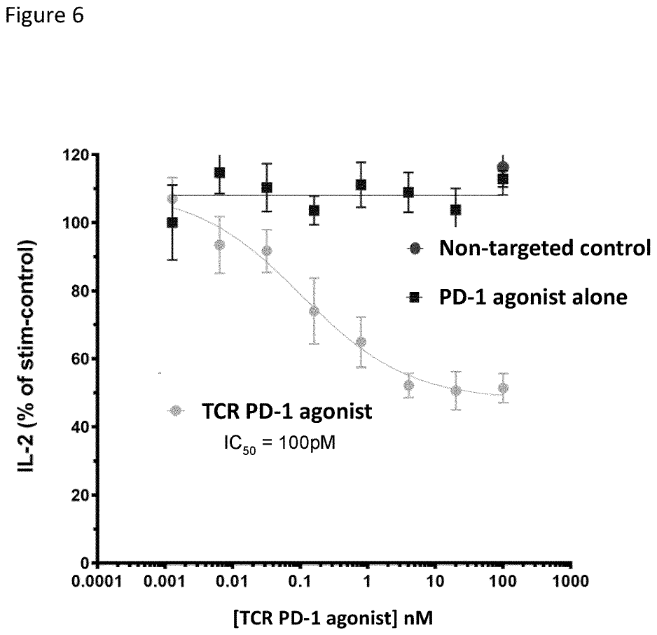

Results demonstrated that in the presence of PPI peptide-pulsed APCs, TCR PD-1 agonist molecules reduced IL-2 production from activated T cells by 40-50%, when present at picomolar levels (FIG. 6). In addition, PD-1 agonist alone, and the non-targeted TCR PD-1 agonist control, did not show a reduction in IL-2 levels, indicating that targeting of the PD-1 agonist to the immune synapse is required for functional activity.

These data demonstrate that targeted TCR PD-1 agonist molecules are potent inhibitors of primary CD4⁺ T cells. Furthermore, the lack of activity seen with non-targeted molecules indicate the potential to avoid the risk of systemic activation in vivo.

b) Protection of Pancreatic β-Cell Co-Cultured with Autoreactive T Cells

TCR PD-1 agonist binding molecules as described in Example 4 were tested to determine their ability to inhibit killing of the pancreatic β-cell line EndoCβH2-A2 and cytokine release by autoreactive CD8⁺ T cells.

EndoC-βH2 target cells labelled with mKate 2 (EndoC-βH2 Red) were generated by transducing EndoC-βH2 cells with HLA-A*02 B2-microglobulin lentivirus construct and NucLight red lentivirus reagent (Sartorius). Target cells were plated at 5×10⁴ cells per well of a 96 well plate in Optiβ3 media, incubated over night at 37° C. 5% CO₂. TCR PD-1 agonist molecules, or control molecules, were added at different concentrations and incubated for 2 hours. To initiate the assay, one of two β-cell specific CD8⁺ T cell clones, having high or low affinity for target cells, was added to EndoC-βH2 red target cells at 5×10⁴ cells per well. PD-L1 transduced EndoC-βH2 red target cells+/−anti-PD-L1 blocking antibody were used as additional controls. Cell killing was determined by quantification of EndoC-βH2 red cell number over time using the IncuCyte S3 imaging system (Sartorius). The number of red nucleus-labelled cells at each time point was normalised to the initial number of objects to take in account variation in cell density in the area visualised. The number of events were acquired in four images and averaged. Cytokine release was measured by V-PLEX Plus Proinflammatory Panel 1 (human) kit in accordance with the manufacturer's instructions (MSD, Meso Scale Diagnostics) using culture supernatants from the IncuCyte killing assays at 24 hours after time point. For the cytokine assay non-stimulated T cells alone were assessed as additional controls. Cytokine release was normalised against stimulated controls and dose response data was analyzed in Prism (GraphPad) using a four parameter, non-linear least squares fit to determine IC₅₀ values.

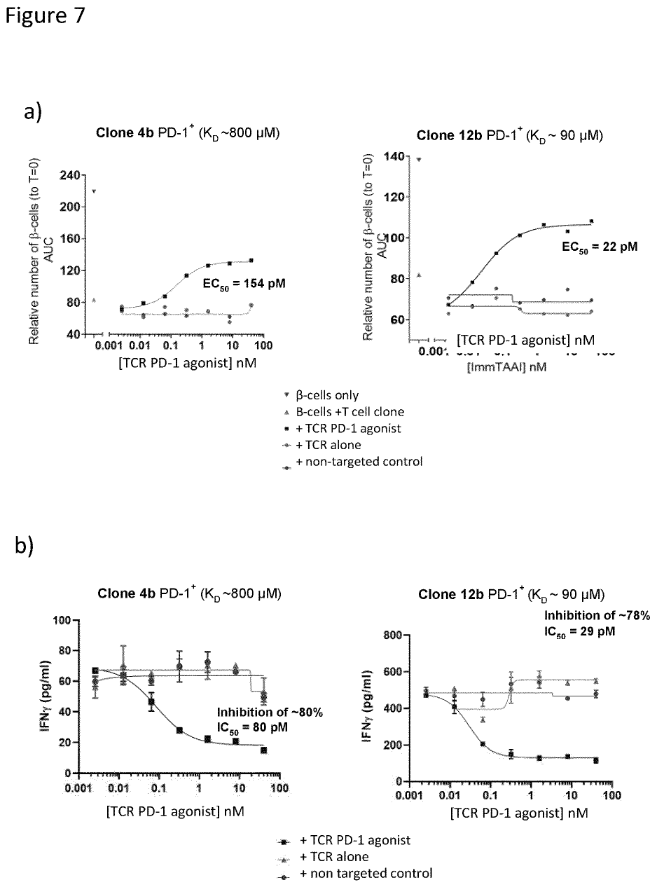

Data showed that the relative number of B cells, when co-cultured in the presence of autoreactive T cells, increased in a dose dependent manner with increasing concentrations of TCR PD-1 agonist binding molecule, thereby demonstrating that the molecules can protect B cells from killing by autoreactive T cells. No effect was observed with the PPI TCR alone or with a non-targeted control (FIG. 7a).

Cell culture supernatants from both co-culture assays were assessed for cytokine production and showed that TCR PD-1 agonist potently inhibits IFNγ production by autoreactive T cells (FIG. 7b).

These data demonstrate that TCR PD-1 agonist binding molecules inhibit killing and cytokine release by T cells that bookend the anticipated affinity range of the natural repertoire of autoreactive T cells, indicating the therapeutic potential of the molecule.

c) Suppression of PD-1^{+ve} NK cell stimulation

TCR PD-1 agonist binding molecules as described in Example 4 were further investigated to determine their ability to inhibit stimulation of PD-1^{+ve} NK cells. To explore if TCR PD-1 agonist can specifically inhibit PD-1+ NK cells, NK cells were activated with the pancreatic β cell line EndoC-βH2. Activation was monitored by expression of cytotoxicity marker CD107a and IFNγ production.

Primary human NK cells were isolated from PBMCs using a NK cell isolation kit (Miltenyi Biotec 130-092-657). The NK cells were incubated 6 days in R10 medium (RPMI-1640 supplemented with 10% heat-inactivated FBS, 2 mM L-glutamine, 1 mM sodium pyruvate) with Dexamethasone (500 ng/ml, Merck, D2915), IL-12 (10 ng/ml, Miltenyi Biotec 130-096-704) IL-15 (25 ng/mL, Peprotech) and IL-18 (100 ng/ml, R&D systems, 9124-IL-050). After 6 days, NK cells were washed in R10 and incubated with or without the TCR-PD1 agonist binding molecule for 4 h (37C, 5% CO₂) with EndoC-βH2 HLA-A*02+ cell at ratio (effector/target) 1/4 in R10 with monensin, brefeldin A (GolgiPlug and GolgiStop BD) and Anti-CD107a antibody. After activation, NK cells were subject to surface staining (Anti-CD56, Anti-CD3, Anti-PD1 and dead cell marker) for 30 min, then fixed and permeabilized (eBioscience Foxp3 Transcription Factor Staining Buffer Set Cat: 00-5523-00) for IFNγ intra cellular staining.

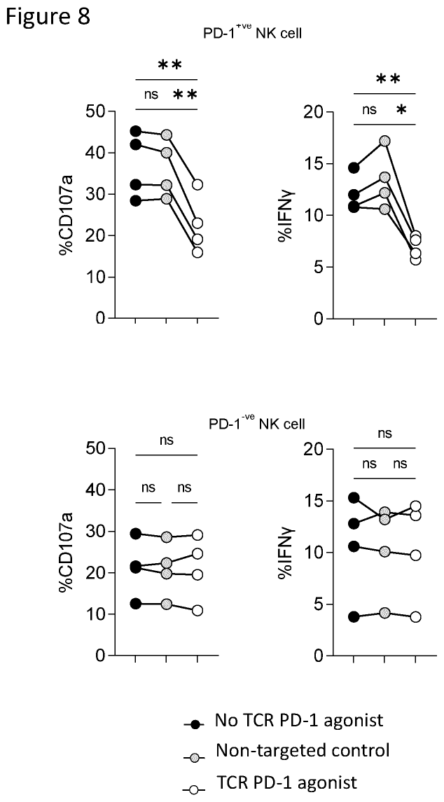

Data showed that in presence of TCR PD-1 agonist the level CD107 and IFNγ expression decreased in the PD-1+ve NK cells. No effect was observed on PD-1−ve NK cells. Thus, TCR PD-1 agonist specifically decreases PD-1+NK cell activation (FIG. 8). Data shown were obtained from 2 independent experiments.

These data demonstrate that TCR PD-1 agonist binding molecules inhibit stimulation of PD-1+ve NK cells. This provides a potential additional therapeutic mechanism of action and could provide differentiation from other approaches.

In total, these data obtained across various disease relevant models, evidence the therapeutic potential of the TCR-PD-1 agonist molecules for the treatment of T1D.

### Example 7—Further Evidence that TCR-PD-1 Agonist Molecules Suppress PD-1+ NK Cell Effector Function

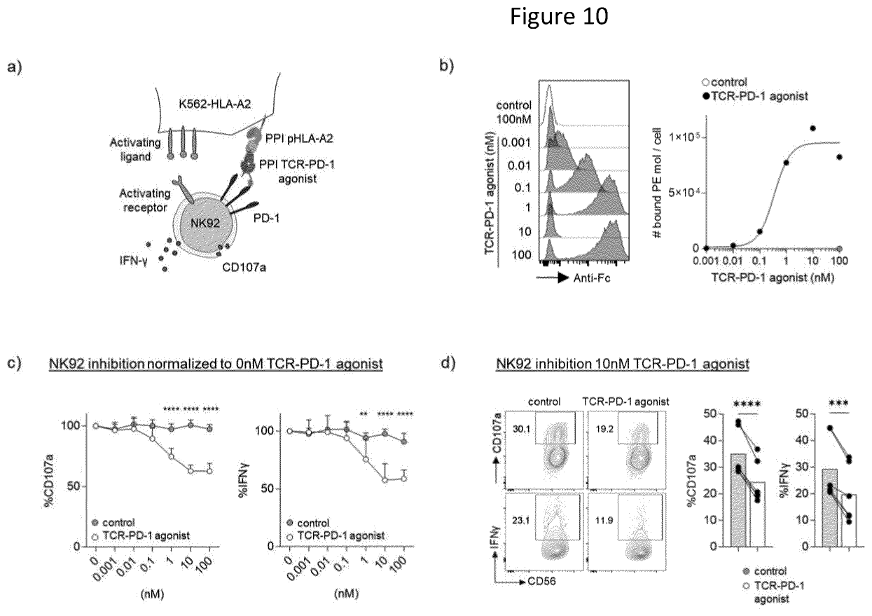

NK cells can infiltrate Type 1 diabetic pancreas and kill human pancreatic β cells. To further investigate the mode of action of TCR-PD-1 agonist molecules an established in vitro model of NK effector function was employed. The NK cell line NK92, expressing PD-1, was activated with K562 lymphoblast cells transduced with HLA-A*02 and pulsed with PPI peptide (FIG. 10a). NK cell activation was measured in the presence of either TCR-PD-1 agonist molecule as described in Example 4, or a control molecule comprising an irrelevant TCR fused to PD-1 agonist. NK cell activation was assessed by monitoring IFNγ production and CD107 degranulation marker expression.

Briefly, HLA-A*02+ve K562 target cells were used as target cells and labelled with Cell Tracker Orange (Invitrogen) for 30 min, washed and resuspended in SCGM medium 20% FBS. Target cells were loaded with PPI peptide ALWGPDPAAA (SEQ ID NO: 1) at 20 μM for 1 h and subsequently incubated for 1 h with either TCR-PD-1 agonist molecule, a18b16, or a control-molecule, at the indicated final concentration. TCR-PD-1 agonist binding was confirmed with PE-conjugated goat anti-human IgG Fc. The number of bound molecules was calculated using a PE Quantitation kit (BD Bioscience) (FIG. 10b). NK92-PD-1+ cells were added to the targets with monensin, brefeldin A (GolgiPlug 1/500 and GolgiStop 1/750 BD) and Anti-CD107a antibody, and incubated for 4 h (37° C., 5% CO₂). After activation, NK92-PD-1+ cells were subject to surface staining (Anti-CD56-BB700, Anti-PD-1-PE-Cy7 and dead cell marker-Pacific-Orange) for 30 min, then fixed and permeabilized (eBioscience Foxp3 Transcription Factor Staining Buffer Set Cat: 00-5523-00) for IFNγ intra cellular staining.

TCR-PD-1 agonist molecule was found to suppress PD-1+NK cell activation in a dose dependent manner while no effect was observed with the control molecule (Figures c and d). TCR-PD-1 agonist significantly decreased IFNγ production (from 29% to 19%) and CD107 expression (from 35% to 24%) at a concentration of 10 nM after only four hours activation.

These data, obtained using an established model of NK cell effector function, further demonstrate that TCR-PD-1 agonist molecules can down regulate NK cell effector function in a specific and concentration dependent manner, and therefore may provide an additional therapeutic mechanism of action for the treatment of T1D.

### Example 8—TCR-PD-1 Agonist Molecules Confer a Prolonged Exhaustion-Like Phenotype to CD4 T Cells

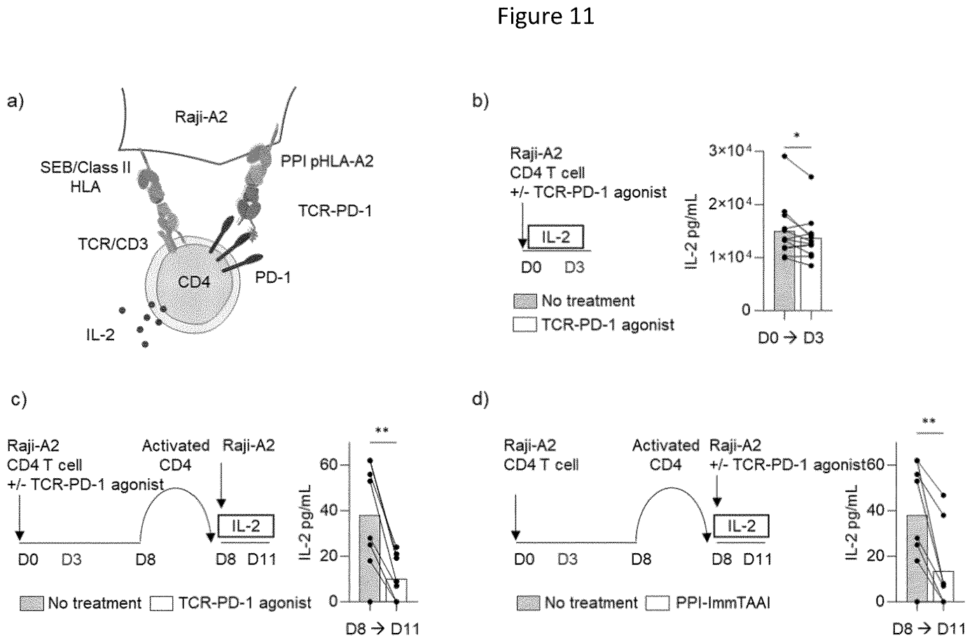

To further investigate the effect of TCR-PD-1 agonist on modulation of the T cell response purified human CD4 T cells were activated with irradiated Raji-HLA-A2 loaded with SEB superantigen and PPI peptide and incubated with TCR-PD-1 agonist molecule described in Example 4. Reduction in IL2 release was used as a marker of T cell exhaustion (FIG. 11a).

Briefly, human primary CD4 T cells were isolated from frozen PBMCs using a CD4 T cell isolation kit (Miltenyi Biotec) and resuspended at 2×10⁶ cell/mL in R10 medium (RPMI-1640 supplemented with 10% heat-inactivated FBS, 2 mM L-glutamine, 1 mM sodium pyruvate). Raji-HLA-A2 were loaded with SEB peptide (100 ng/ml List Labs) and PPI peptide (40 μM ALWGPDPAAA) for 1 h (37° C., 5% CO₂) in R10 medium at 2.106 cell/mL then irradiated at 60 Gray. Irradiated Raji-HLA-A2 were then incubated in the presence or absence of 20 nM TCR-PD-1 agonist, a19b20, for 1 h (37° C., 5% CO₂). CD4 T cells were activated for 8 days with irradiated Raji-HLA-A2 (ratio 1:1 of effectors to Raji cells and 10 nM TCR-PD-1 agonist). At day 8, activated CD4 T cells were washed 2 times with R10 medium and re-incubated for 3 days with new irradiated Raji-HLA-A2 prepared as before. Culture supernatant was collected at day 3 during CD4 T cell activation and day 11. IL-2 produced by CD4⁺ T cells from day 0 to day 3 and day 8 to day 11 was measured by ELISA (Invitrogen).

TCR-PD-1 agonist activated the PD-1 pathway on interacting CD4 T cells and achieved immune suppression during T cell priming as shown by the decrease in IL-2 secretion at day 3 (FIG. 11b). Prolonged CD4 T cell activation induced an exhaustion-like phenotype in the absence of TCR-PD-1 agonist as shown by the decrease in IL-2 concentration from 15000 μg/mL at day 3 to 38 μg/mL upon re-activation (FIGS. 11b and c, mean, no treatment). Importantly, the reduction in IL2 was further enhanced when TCR-PD-1 agonist was present prior to re-activation (FIG. 11c). Furthermore, addition of TCR-PD-1 agonist after reactivation also enhances the reduction in IL2 production, indicating the potential for ongoing immune modulation (FIG. 11d).

These data show that TCR-PD-1 agonist molecules of invention have the potential to confer an enhanced and prolonged down modulation of the CD4 T cell response in vivo, which could provide a novel therapeutic mechanism of action for the treatment of T1D.

1. A binding molecule comprising a peptide-major histocompatibility complex (pMHC)-binding domain that has the property of binding to a ALWGPDAAA (SEQ ID NO: 1) HLA-A*02 complex, wherein the pMHC-binding domain comprises (i) an alpha chain, comprising at least a TCR alpha chain variable domain, and (ii) a beta chain, comprising at least a TCR beta chain variable domain, wherein
(a) the TCR alpha chain variable domain comprises a CDR1, a CDR2 and a CDR3 comprising the following sequences:
CDR1—DKHSQG (SEQ ID NO: 23), optionally with one, two or three mutations therein,
CDR2—IYSQGD (SEQ ID NO: 27), optionally with one, two or three mutations therein,
CDR3—AVRGNEKLT (SEQ ID NO: 7), optionally with one, two or three mutations therein, and/or
(b) the TCR beta chain variable domain comprises a CDR1, a CDR2 and a CDR3 comprising the following sequences:
CDR1—LQHSY (SEQ ID NO: 35), optionally with one, two or three mutations therein,
CDR2—SVGVGF (SEQ ID NO: 29), optionally with one, two or three mutations therein,
CDR3—ASAYMTGELF (SEQ ID NO: 30), optionally with one, two or three mutations therein.

2. The binding molecule of claim 1, wherein the mutation(s) in the TCR alpha chain variable domain CDRs are selected from K28R (CDR1), H29G (CDR1), G32S (CDR1) and Q53N (CDR2), numbered according to SEQ ID NO: 26; and/or the mutation(s) in the beta chain CDRs are selected from L27M (CDR1), Q28N (CDR1), S30N (CDR1), V52A (CDR2), F54I (CDR2) and A104S (CDR3), numbered according to SEQ ID NO: 74.

3. The binding molecule of claim 1 or claim 2, comprising one of the following combinations of TCR alpha chain variable domain CDRs and TCR beta chain variable domain CDRs:
(a) alpha chain CDR1, CDR2 and CDR3 amino acid sequences of DRGSQS (SEQ ID NO: 5), IYSNGD (SEQ ID NO: 6) and AVRGNEKLT (SEQ ID NO: 7), respectively, and beta chain CDR1, CDR2 and CDR3 amino acid sequences of MNHNY (SEQ ID NO: 15), SVGAGI (SEQ ID NO: 16) and ASSYMTGELF (SEQ ID NO: 17), respectively;
(b) alpha chain CDR1, CDR2 and CDR3 amino acid sequences of DKHSQG (SEQ ID NO: 23), IYSNGD (SEQ ID NO: 6) and AVRGNEKLT (SEQ ID NO: 7), respectively, and beta chain CDR1, CDR2 and CDR3 amino acid sequences of MNHSY (SEQ ID NO: 28), SVGVGF (SEQ ID NO: 29) and ASAYMTGELF (SEQ ID NO: 30), respectively;
(c) alpha chain CDR1, CDR2 and CDR3 amino acid sequences of DKHSQG (SEQ ID NO: 23), IYSNGD (SEQ ID NO: 6) and AVRGNEKLT (SEQ ID NO: 7), respectively, and beta chain CDR1, CDR2 and CDR3 amino acid sequences of MQHSY (SEQ ID NO: 32), SVGVGF (SEQ ID NO: 29) and ASAYMTGELF (SEQ ID NO: 30), respectively; or
(d) alpha chain CDR1, CDR2 and CDR3 amino acid sequences of DKHSQG (SEQ ID NO: 23), IYSQGD (SEQ ID NO: 27) and AVRGNEKLT (SEQ ID NO: 7), respectively, and beta chain CDR1, CDR2 and CDR3 amino acid sequences of LQHSY (SEQ ID NO: 35), SVGVGF (SEQ ID NO: 29) and ASAYMTGELF (SEQ ID NO: 30), respectively.

4. The binding molecule of any preceding claim, wherein the TCR alpha chain variable domain comprises framework regions, FR1, FR2, FR3 and FR4, comprising the following sequences:
FR1—AKEVEQNSGPLSVPEGAIASLQCTYS (SEQ ID NO: 25), optionally with one, two or three mutations therein,
FR2—FFWYRQYSGKSPELIMS (SEQ ID NO: 9), optionally with one, two or three mutations therein,
FR3—KEDGRFTAQLNKASQYVSLLIRDSQPSDSATYLC (SEQ ID NO: 10), optionally with one, two or three mutations therein,
FR4—FGTGTRLTIIP (SEQ ID NO: 11), optionally with one, two or three mutations therein, and/or the TCR beta chain variable domain comprises framework regions, FR1, FR2, FR3 and FR4, comprising the following sequences:
FR1—NAGVTQTPKFRILKIGQSMTLQCAQD (SEQ ID NO: 18), optionally with one, two or three mutations therein,
FR2—MYWYRQDPGMGLKPIYY (SEQ ID NO: 19), optionally with one, two or three mutations therein,
FR3—TDKGEVPQGYQVSRSTTEDFPLRLESAAPSQTSVYFC (SEQ ID NO: 75), optionally with one, two or three mutations therein,
FR4—FGEGSRLTVL (SEQ ID NO: 21), optionally with one, two or three mutations therein.

5. The binding molecule of claim 4, wherein the mutation(s) in the TCR alpha chain variable domain framework regions are selected from A1Q and Q22N, numbered according to SEQ ID NO: 26, and/or the mutation(s) in the TCR beta chain variable domain framework regions are selected from Q62N, Q62E, Q62D and Q65N, numbered according to SEQ ID NO: 74.

6. The binding molecule of any preceding claim, wherein
(a) the TCR alpha chain variable domain comprises an amino acid sequence provided in any one of SEQ ID NOs: 3, 22, 24 and 26, or an amino acid sequence having at least 80%, at least 90%, at least 95% or at least 98% identity to any one of SEQ ID NOs: 3, 22, 24 and 26, and
(b) the TCR beta chain variable domain comprises an amino acid sequence provided in any one of SEQ ID NOS: 13, 68, 31, 34, 74, 76 and 78, or an amino acid sequence having at least 80%, at least 90%, at least 95% or at least 98% identity to any one of SEQ ID NOs: 13, 68, 31, 34, 74, 76 and 78.

7. The binding molecule of any one of the preceding claims, comprising one of the following combinations of TCR alpha and beta chain variable domains:
(a) a TCR alpha chain variable domain comprising the amino acid sequence provided in SEQ ID NO: 22 and a TCR beta chain variable domain comprising the amino acid sequence provided in SEQ ID NO: 68;
(b) a TCR alpha chain variable domain comprising the amino acid sequence provided in SEQ ID NO: 24 and a TCR beta chain variable domain comprising the amino acid sequence provided in SEQ ID NO: 31;
(c) a TCR alpha chain variable domain comprising the amino acid sequence provided in SEQ ID NO: 26 and a TCR beta chain variable domain comprising the amino acid sequence provided in SEQ ID NO: 34;
(d) a TCR alpha chain variable domain comprising the amino acid sequence provided in SEQ ID NO: 26 and a TCR beta chain variable domain comprising the amino acid sequence provided in SEQ ID NO: 74;
(e) a TCR alpha chain variable domain comprising the amino acid sequence provided in SEQ ID NO: 26 and a TCR beta chain variable domain comprising the amino acid sequence provided in SEQ ID NO: 76; or
(f) a TCR alpha chain variable domain comprising the amino acid sequence provided in SEQ ID NO: 26 and a TCR beta chain variable domain comprising the amino acid sequence provided in SEQ ID NO: 78.

8. The binding molecule of any preceding claim, wherein the TCR alpha chain variable domain comprises the amino acid sequence of SEQ ID NO: 26 and the TCR beta chain variable domain comprises the amino acid sequence of SEQ ID NO: 74.

9. The binding molecule of any preceding claim, wherein the alpha chain comprises a TCR alpha chain constant domain and/or the beta chain comprises a TCR beta chain constant domain.

10. The binding molecule of claim 9, wherein a non-native disulphide bond links a residue of the TCR alpha chain constant domain to a residue of the TCR beta chain constant domain.

11. The binding molecule of claim 9 or claim 10, wherein
the TCR alpha chain constant domain comprises the amino acid sequence provided in SEQ ID NO: 37, or an amino acid sequence having at least 80%, at least 90%, at least 95% or at least 98% identity to SEQ ID NO: 37; and/or
the TCR beta chain constant domain comprises the amino acid sequence provided in SEQ ID NO: 39, or an amino acid sequence having at least 80%, at least 90%, at least 95% or at least 98% identity to SEQ ID NO: 39.

12. The binding molecule of any preceding claim, which comprises two or more polypeptide chains, wherein the alpha chain and the beta chain are comprised in separate polypeptide chains.

13. The binding molecule of any preceding claim, wherein
(a) the alpha chain comprises the amino acid sequence provided in any one of SEQ ID NOs: 2, 70, 36, and 40, or an amino acid sequence that has at least 90%, at least 95%, or at least 98% identity to an amino acid sequence provided in any one of SEQ ID NOs: 2, 70, 36, and 40, and
(b) the beta chain comprises the amino acid sequence provided in any one of SEQ ID NOs: 12, 69, 38, 41, 80, 81 and 82, or an amino acid sequence that has at least 90%, at least 95%, or at least 98% identity to the amino acid sequence provided in any one of SEQ ID NOs: 12, 69, 38, 41, 80, 81 and 82.

14. The binding molecule of any preceding claim, wherein
(a) the alpha chain comprises the amino acid sequence of SEQ ID NO: 70 and the beta chain comprises the amino acid sequence of SEQ ID NO: 69;
(b) the alpha chain comprises the amino acid sequence of SEQ ID NO: 36 and the beta chain comprises the amino acid sequence of SEQ ID NO: 38;
(c) the alpha chain comprises the amino acid sequence of SEQ ID NO: 40 and the beta chain comprises the amino acid sequence of SEQ ID NO: 41;
(d) the alpha chain comprises the amino acid sequence of SEQ ID NO: 40 and the beta chain comprises the amino acid sequence of SEQ ID NO: 80;
(e) the alpha chain comprises the amino acid sequence of SEQ ID NO: 40 and the beta chain comprises the amino acid sequence of SEQ ID NO: 81; or
(f) the alpha chain comprises the amino acid sequence of SEQ ID NO: 40 and the beta chain comprises the amino acid sequence of SEQ ID NO: 82.

15. The binding molecule of any one of claims 1 to 11, wherein the pMHC binding domain is in a single polypeptide chain format of the type Vα-L-Vβ, Vβ-L-Vα, Vα-Ca-L-Vβ, or Vα-L-Vβ-Cβ, wherein Vα and Vβ are TCR α and β variable regions respectively, Cα and Cβ are TCR α and β constant regions respectively, and L is a linker sequence.

16. The binding molecule of any preceding claim, further comprising an immune suppressor.

17. The binding molecule of claim 16, wherein the immune suppressor is an immune checkpoint agonist, optionally a PD-1 agonist.

18. The binding molecule of any preceding claim, wherein the immune suppressor comprises an antigen binding moiety that is capable of binding to an antigen.

19. The binding molecule of claim 18, wherein the antigen is PD-1 and the antigen binding moiety is a PD-1 agonist.

20. The binding molecule of claim 18 or claim 19, wherein the antigen binding moiety comprises an antibody or antigen binding fragment thereof.

21. The binding molecule of any one of claims 18 to 20, wherein the antigen binding moiety comprises a single domain antibody, optionally a VHH.

22. The binding molecule of claim 21, wherein the single domain antibody binds to PD-1 and comprises CDRs, CDR1, CDR2 and CDR3, having the following amino acid sequences:
CDR1—GFTFSSYA (SEQ ID NO: 43), optionally with one, two or three mutations therein,
CDR2—IASDGAST (SEQ ID NO: 44), optionally with one, two or three mutations therein, and
CDR3—CARGGYLTYDRY (SEQ ID NO: 45), optionally with one, two or three mutations therein.

23. The binding molecule of claim 21 or claim 22, wherein the single domain antibody is a VHH comprising the amino acid sequence of SEQ ID NO: 42, or a humanised version thereof, or an amino acid sequence having at least 80%, at least 90%, at least 95% or at least 98% identity to SEQ ID NO: 42.

24. The binding molecule of claim 21 or 22, wherein the single domain antibody is a VHH comprising the amino acid sequence of SEQ ID NO: 71, or an amino acid sequence having at least 80%, at least 90%, at least 95% or at least 98% identity to SEQ ID NO: 71.

25. The binding molecule of any one of claims 16 to 24, wherein the immune suppressor is covalently linked to the pMHC binding domain via the C- or N-terminus of the alpha or beta chain, optionally via a linker sequence, further optionally wherein the linker sequence is selected from SEQ ID NOs: 55 to 66 or 73.

26. The binding molecule of any one of claims 16 to 25, comprising
(a) a first polypeptide chain comprising the immune suppressor and the beta chain of the pMHC binding domain; and
(b) a second polypeptide chain comprising the alpha chain of the pMHC-binding domain.

27. The binding molecule of claim 26, wherein the C-terminus of the immune suppressor is covalently linked to the N-terminus of the TCR beta chain, optionally via the linker sequence of SEQ ID NO: 73.

28. The binding molecule of claim 26 or claim 27, wherein
(a) the first polypeptide comprises an amino acid sequence provided in any one of SEQ ID NOs: 72, 46, 47, 83, 84 and 85, or an amino acid sequence that has at least 90%, at least 95%, or at least 98% identity to an amino acid sequence provided in any one of SEQ ID NOs: 72, 46, 47, 83, 84 and 85, and
(b) the second polypeptide comprises an amino acid sequence provided in any one of SEQ ID NOs: 70, 36, and 40, or an amino acid sequence that has at least 90%, at least 95%, or at least 98% identity to an amino acid sequence provided in any one of SEQ ID NOs 70, 36, and 40.

29. The binding molecule of any one of claims 26 to 28, wherein
(a) the first polypeptide chain comprises the amino acid sequence of SEQ ID NO: 72 and the second polypeptide chain comprises the amino acid sequence of SEQ ID NO: 70;
(b) the first polypeptide chain comprises the amino acid sequence of SEQ ID NO: 46 and the second polypeptide chain comprises the amino acid sequence of SEQ ID NO: 36;
(c) the first polypeptide chain comprises the amino acid sequence of SEQ ID NO: 47 and the second polypeptide chain comprises the amino acid sequence of SEQ ID NO: 40;
(d) the first polypeptide chain comprises the amino acid sequence of SEQ ID NO: 83 and the second polypeptide chain comprises the amino acid sequence of SEQ ID NO: 40;
(e) the first polypeptide chain comprises the amino acid sequence of SEQ ID NO: 84 and the second polypeptide chain comprises the amino acid sequence of SEQ ID NO: 40; or
(f) the first polypeptide chain comprises the amino acid sequence of SEQ ID NO: 85 and the second polypeptide chain comprises the amino acid sequence of SEQ ID NO: 40.

30. The binding molecule of any preceding claim, further comprising a half-life extending domain, optionally comprising a first portion of an IgG Fc region (FC1) and a second portion of an IgG Fc region (FC2).

31. The binding molecule of any one of claims 16 to 29, further comprising a half-life extending domain comprising a first portion of an IgG Fc region (FC1) and a second portion of an IgG Fc region (FC2), wherein the binding molecule comprises
(a) a first polypeptide chain comprising the immune suppressor and either (i) the alpha chain or (ii) beta chain of the pMHC binding domain;
(b) a second polypeptide chain comprising FC1 and the other of (i) the alpha chain and (ii) the beta chain; and
(c) a third polypeptide chain comprising FC2.

32. The binding molecule of claim 31, wherein the C-terminus of the immune suppressor is covalently linked to the N-terminus of either (i) the alpha chain or (ii) beta chain of the pMHC binding domain, optionally via the linker sequence of SEQ ID NO: 73.

33. The binding molecule of claim 31 or claim 32, wherein the C-terminus of the other of (i) the alpha chain and (ii) the beta chain is covalently linked to the N-terminus of FC1 via an IgG hinge sequence, and/or wherein the third polypeptide comprises an IgG hinge sequence at the N-terminus of FC2, optionally wherein the IgG hinge comprises the amino acid sequence of SEQ ID NO: 50.

34. The binding molecule of any one of claims 31 to 33, wherein the first polypeptide chain comprises the beta chain of the pMHC binding domain and the second polypeptide chain comprises the alpha chain of the pMHC binding domain.

35. The binding molecule of any one of claims 30 to 34, wherein the half-life extending domain comprises
(a) one or more amino acid substitutions which facilitate dimerisation of FC1 and FC2; and/or
(b) one or more amino acid substitutions which prevent or reduce binding to FcγR; and/or
(c) one or more amino acid substitutions which promote binding to FcRn.

36. The binding molecule of any one of claims 30 to 35, wherein
(a) either FC1 or FC2 comprises the amino acid sequence provided in SEQ ID NO: 49 or 93, or an amino acid sequence that has at least 90%, at least 95%, or at least 98% identity to the amino acid sequence provided in SEQ ID NO: 49 or 93, and
(b) the other of FC1 and FC2 comprises the amino acid sequence provided in SEQ ID NO: 52 or 94, or an amino acid sequence that has at least 90%, at least 95%, or at least 98% identity to the amino acid sequence provided in SEQ ID NO: 52 or 94.

37. The binding molecule of any one of claims 31 to 36, wherein
(a) the first polypeptide comprises an amino acid sequence provided in any one of SEQ ID NOs: 72, 46, 47, 83, 84 and 85, or an amino acid sequence that has at least 90%, at least 95%, or at least 98% identity to an amino acid sequence provided in any one of SEQ ID NOs: 72, 46, 47, 83, 84 and 85;
(b) the second polypeptide comprises an amino acid sequence provided in any one of SEQ ID NOs: 48, 53, 54, 97, 99 or 100, or an amino acid sequence that has at least 90%, at least 95%, or at least 98% identity to an amino acid sequence provided in any one of SEQ ID NOs: 48, 53, 54, 97, 99 or 100; and
(c) the third polypeptide chain comprises the amino acid sequence of SEQ ID NO: 51 or 98, or an amino acid sequence that has at least 90%, at least 95%, or at least 98% identity to the amino acid sequence of SEQ ID NO: 51 or 98.

38. The binding molecule of any one of claims 31 to 37, wherein
(a) the first polypeptide chain comprises the amino acid sequence of SEQ ID NO: 72, the second polypeptide chain comprises the amino acid sequence of SEQ ID NO: 48 and the third polypeptide chain comprises the amino acid sequence of SEQ ID NO: 51;
(b) the first polypeptide chain comprises the amino acid sequence of SEQ ID NO: 46, the second polypeptide chain comprises the amino acid sequence of SEQ ID NO: 53 and the third polypeptide chain comprises the amino acid sequence of SEQ ID NO: 51;
(c) the first polypeptide chain comprises the amino acid sequence of SEQ ID NO: 47, the second polypeptide chain comprises the amino acid sequence of SEQ ID NO: 54 and the third polypeptide chain comprises the amino acid sequence of SEQ ID NO: 51;
(d) the first polypeptide chain comprises the amino acid sequence of SEQ ID NO: 83, the second polypeptide chain comprises the amino acid sequence of SEQ ID NO: 54 and the third polypeptide chain comprises the amino acid sequence of SEQ ID NO: 51;
(e) the first polypeptide chain comprises the amino acid sequence of SEQ ID NO: 84, the second polypeptide chain comprises the amino acid sequence of SEQ ID NO: 54 and the third polypeptide chain comprises the amino acid sequence of SEQ ID NO: 51;
(f) the first polypeptide chain comprises the amino acid sequence of SEQ ID NO: 85, the second polypeptide chain comprises the amino acid sequence of SEQ ID NO: 54 and the third polypeptide chain comprises the amino acid sequence of SEQ ID NO: 51;
(g) a first polypeptide chain comprising the amino acid sequence of SEQ ID NO: 72, a second polypeptide chain comprising the amino acid sequence of SEQ ID NO: 97 and a third polypeptide chain comprising the amino acid sequence of SEQ ID NO: 98;
(h) a first polypeptide chain comprising the amino acid sequence of SEQ ID NO: 46, a second polypeptide chain comprising the amino acid sequence of SEQ ID NO: 99 and a third polypeptide chain comprising the amino acid sequence of SEQ ID NO: 98;
(i) a first polypeptide chain comprising the amino acid sequence of SEQ ID NO: 47, a second polypeptide chain comprising the amino acid sequence of SEQ ID NO: 100 and a third polypeptide chain comprising the amino acid sequence of SEQ ID NO: 98;
(j) a first polypeptide chain comprising the amino acid sequence of SEQ ID NO: 83, a second polypeptide chain comprising the amino acid sequence of SEQ ID NO: 100 and a third polypeptide chain comprising the amino acid sequence of SEQ ID NO: 98;
(k) a first polypeptide chain comprising the amino acid sequence of SEQ ID NO: 84, a second polypeptide chain comprising the amino acid sequence of SEQ ID NO: 100 and a third polypeptide chain comprising the amino acid sequence of SEQ ID NO: 98; or
(l) a first polypeptide chain comprising the amino acid sequence of SEQ ID NO: 85, a second polypeptide chain comprising the amino acid sequence of SEQ ID NO: 100 and a third polypeptide chain comprising the amino acid sequence of SEQ ID NO: 98.

39. The binding molecule of any one of claims 31 to 37, wherein the first polypeptide chain comprises the amino acid sequence of SEQ ID NO: 83, the second polypeptide chain comprises the amino acid sequence of SEQ ID NO: 54 or 100 and the third polypeptide chain comprises the amino acid sequence of SEQ ID NO: 51 or 98.

40. The binding molecule of any preceding claim, wherein the pMHC-binding domain binds to the ALWGPDPAAA (SEQ ID NO: 1)-HLA-A*02 complex with an affinity which is at least at least 10-fold, at least 100-fold, at least 500-fold, or at least 1000-fold higher than its affinity for a ALLGPDPAAA (SEQ ID NO: 67)-HLA-A*02 complex.

41. A single domain antibody that binds to PD-1 comprising a CDR1, CDR2 and CDR3, having the following amino acid sequences:
CDR1—GFTFSSYA (SEQ ID NO: 43), optionally with one, two or three mutations therein;
CDR2—IASDGAST (SEQ ID NO: 44), optionally with one, two or three mutations therein; and
CDR3—CARGGYLTYDRY (SEQ ID NO: 45), optionally with one, two or three mutations therein.

42. The single domain antibody of claim 41, wherein
(a) the single domain antibody is isolated; and/or
(b) the single domain antibody is a VHH; and/or
(c) the single domain antibody is a PD-1-agonist; and/or
(d) the single domain antibody binds to PD-1 with a K_{D} in the range of about 1 nM to about 500 nM or in the range of about 50 nM to about 70 nM; and/or
(e) the single domain antibody binds to an epitope in PD-1 comprising one or more or all of the following amino acids: E38, F59, P60, E61, T75, Q76, L77, P78, N79 and G80, numbered according to SEQ ID NO: 101; and/or
(f) the single domain antibody comprises the amino acid sequence provided in SEQ ID NO: 42 or a humanised version thereof, or an amino acid sequence having at least 80%, at least 90%, at least 95% or at least 98% identity to SEQ ID NO: 42; and/or
(g) the single domain antibody comprises the amino acid sequence of SEQ ID NO: 71, or an amino acid sequence having at least 80%, at least 90%, at least 95% or at least 98% identity to SEQ ID NO: 71.

43. A binding molecule comprising
(a) the single domain antibody of claim 41 or claim 42;
(b) a pMHC-binding domain, optionally comprising (i) an alpha chain, comprising at least a TCR alpha chain variable domain, and (ii) a beta chain, comprising at least a TCR beta chain variable domain; and
(c) optionally, a half-life extending domain.

44. A nucleic acid encoding
(a) the binding molecule of any one of claim 1 to 30 or 40, wherein the alpha and beta chains are encoded within a single open reading frame, or within two distinct open reading frames;
(b) the binding molecule of any one of claims 31 to 39, wherein the first, second and third polypeptide chains are encoded within a single open reading frame, or within distinct open reading frames; and/or
(c) the single domain antibody of claim 41 or claim 42 or the binding molecule of claim 43.

45. An expression vector comprising the nucleic acid of claim 44.

46. A cell harbouring
(a) the expression vector of claim 45;
(b) a first expression vector comprising a nucleic acid encoding a first polypeptide comprising the beta chain of the binding molecule of any one of claim 1 to 30 or 40 and a second expression vector comprising a nucleic acid encoding a second polypeptide comprising the alpha chain of the binding molecule of any one of claim 1 to 30 or 40; or
(c) a first expression vector comprising a nucleic acid encoding the first polypeptide of the binding molecule of any one of claims 31 to 39, a second expression vector comprising a nucleic acid encoding the second polypeptide of the binding molecule of any one of claims 31 to 39, and a third expression vector comprising a nucleic acid encoding the third polypeptide of the binding molecule of any one of claims 31 to 39.

47. A non-naturally occurring and/or purified and/or engineered cell, preferably a T-cell, presenting the binding molecule of any one of claims 1 to 14.

48. A pharmaceutical composition comprising the binding molecule of any one of claim 1 to 40 or 43, the single domain antibody of claim 41 or 42, the nucleic acid of claim 44, the expression vector of claim 45, and/or the cell of claim 46 or 47, together with one or more pharmaceutically acceptable carriers or excipients.

49. The binding molecule of any one of claim 1 to 40 or 43, the single domain antibody of claim 41 or claim 42, the nucleic acid of claim 44, the expression vector of claim 45, and/or the cell of claim 46 or 47, for use in medicine, preferably in a human subject.

50. The binding molecule of any one of claim 1 to 40 or 43, the single domain antibody of claim 41 or claim 42, the nucleic acid of claim 44, the expression vector of claim 45, and/or the cell of claim 46 or 47, for use in a method of treating diabetes, preferably in a human subject.

51. A method of producing the binding molecule of any one of claim 1 to 40 or 43, or the single domain antibody of claim 41 or claim 42, the method comprising a) maintaining a cell according to claim 46 or 47 under optimal conditions for expression of the binding molecule or single domain antibody and b) isolating the binding molecule or single domain antibody.
